SEQUENCE LISTING (80 sequences)

SEQ ID NO: 1 - DNA, 22 bp, Artificial Sequence
  AIM2 (forward primer)
cagaaatgatgtcgcaaagcaa

SEQ ID NO: 2 - DNA, 23 bp, Artificial Sequence
  AIM2 (reverse primer)
tcagtaccataactggcaaacag

SEQ ID NO: 3 - DNA, 20 bp, Artificial Sequence
  BCL2 (forward primer)
gccttctttgagttcggtgg

SEQ ID NO: 4 - DNA, 19 bp, Artificial Sequence
  BCL2 (reverse primer)
atctcccggttgacgctct

SEQ ID NO: 5 - DNA, 21 bp, Artificial Sequence
  BCL2A1 (Bfl-1/A1) (foward primer)
tacaggctggctcaggactat

SEQ ID NO: 6 - DNA, 22 bp, Artificial Sequence
  BCL2A1 (Bfl-1/A1) (reverse primer)
cgcaacattttgtagcactctg

SEQ ID NO: 7 - DNA, 21 bp, Artificial Sequence
  BCL2L1 (BCL-X) (forward primer)
cgacgagtttgaactgcggta

SEQ ID NO: 8 - DNA, 21 bp, Artificial Sequence
  BCL2L1 (BCL-X) (reverse primer)
gggatgtcaggtcactgaatg

SEQ ID NO: 9 - DNA, 22 bp, Artificial Sequence
  BIRC3 (c-IAP1) (forward primer)
aagctacctctcagcctacttt

SEQ ID NO: 10 - DNA, 21 bp, Artificial Sequence
  BIRC3 (c-IAP1) (reverse primer)
ccactgttttctgtacccgga

SEQ ID NO: 11 - DNA, 21 bp, Artificial Sequence
  BMP2 (forward primer)
actaccagaaacgagtgggaa

SEQ ID NO: 12 - DNA, 23 bp, Artificial Sequence
  BMP2 (reverse primer)
catctgttctcggaaaacctgaa

SEQ ID NO: 13 - DNA, 20 bp, Artificial Sequence
  BMP4 (forward primer)
aaagtcgccgagattcaggg

SEQ ID NO: 14 - DNA, 19 bp, Artificial Sequence
  BMP4 (reverse primer)
gacggcactcttgctaggc

SEQ ID NO: 15 - DNA, 19 bp, Artificial Sequence
  BRCA1 (forward primer)
tgtgaggcacctgtggtga

SEQ ID NO: 16 - DNA, 21 bp, Artificial Sequence
  BRCA1 (reverse primer)
cagctcctggcactggtagag

SEQ ID NO: 17 - DNA, 19 bp, Artificial Sequence
  CDKN2A (p16INK4) (forward primer)
atggagccttcggctgact

SEQ ID NO: 18 - DNA, 20 bp, Artificial Sequence
  CDKN2A (p16INK4) (reverse primer)
taactattcggtgcgttggg

SEQ ID NO: 19 - DNA, 20 bp, Artificial Sequence
  CDKN1A (p21CIP1/WAF1) (forward primer)
ggaagaccatgtggacctgt

SEQ ID NO: 20 - DNA, 20 bp, Artificial Sequence
  CDKN1A (p21CIP1/WAF1) (reverse primer)
atgcccagcactcttaggaa

SEQ ID NO: 21 - DNA, 21 bp, Artificial Sequence
  FOS (forward primer)
ggggcaaggtggaacagttat

SEQ ID NO: 22 - DNA, 21 bp, Artificial Sequence
  FOS (reverse primer)
ccgcttggagtgtatcagtca

SEQ ID NO: 23 - DNA, 25 bp, Artificial Sequence
  GATA-4 (forward primer)
gcccaagaacctgaataaatctaag

SEQ ID NO: 24 - DNA, 25 bp, Artificial Sequence
  GATA-4 (reverse primer)
agacatcgcactgactgagaacgtc

SEQ ID NO: 25 - DNA, 20 bp, Artificial Sequence
  ICAM1 (forward primer)
cgtgccgcactgaactggac

SEQ ID NO: 26 - DNA, 21 bp, Artificial Sequence
  ICAM1 (reverse primer)
cctcacacttcactgtcacct

SEQ ID NO: 27 - DNA, 19 bp, Artificial Sequence
  IL10 (forward primer)
aaggcgcatgtgaactccc

SEQ ID NO: 28 - DNA, 20 bp, Artificial Sequence
  IL10 (reverse primer)
acggccttgctcttgttttc

SEQ ID NO: 29 - DNA, 24 bp, Artificial Sequence
  IL6 (forward primer)
aaattcggtacatcctcgacggca

SEQ ID NO: 30 - DNA, 24 bp, Artificial Sequence
  IL6 (reverse primer)
agtgcctctttgctgctttcacac

SEQ ID NO: 31 - DNA, 23 bp, Artificial Sequence
  IL8 (forward primer)
actgagagtgattgagagtggac

SEQ ID NO: 32 - DNA, 21 bp, Artificial Sequence
  IL8 (reverse primer)
aaccctctgcacccagttttc

SEQ ID NO: 33 - DNA, 21 bp, Artificial Sequence
  JUN (forward primer)
tccaagtgccgaaaaaggaag

SEQ ID NO: 34 - DNA, 21 bp, Artificial Sequence
  JUN (reverse primer)
cgagttctgagctttcaaggt

SEQ ID NO: 35 - DNA, 22 bp, Artificial Sequence
  KEAP1 (forward primer)
gtggtgtccattgagggtatcc

SEQ ID NO: 36 - DNA, 19 bp, Artificial Sequence
  KEAP1 (reverse primer)
gctcagcgaagttggcgat

SEQ ID NO: 37 - DNA, 20 bp, Artificial Sequence
  MAP4K4 (forward primer)
gagccacaggtacagtggtc

SEQ ID NO: 38 - DNA, 21 bp, Artificial Sequence
  MAP4K4 (reverse primer)
aagccttttgggtagggtcag

SEQ ID NO: 39 - DNA, 21 bp, Artificial Sequence
  MAPK8 (JNK1) (forward primer)
agaagctaagccgaccatttc

SEQ ID NO: 40 - DNA, 22 bp, Artificial Sequence
  MAPK8 (JNK1) (reverse primer)
tctagggatttctgtggtgtga

SEQ ID NO: 41 - DNA, 19 bp, Artificial Sequence
  MYD88 (forward primer)
ggctgctctcaacatgcga

SEQ ID NO: 42 - DNA, 20 bp, Artificial Sequence
  MYD88 (reverse primer)
tgtccgcacgttcaagaaca

SEQ ID NO: 43 - DNA, 21 bp, Artificial Sequence
  NANOG (forward primer)
gctgagatgcctcacacggag

SEQ ID NO: 44 - DNA, 25 bp, Artificial Sequence
  NANOG (reverse primer)
tctgtttcttgactgggaccttgtc

SEQ ID NO: 45 - DNA, 22 bp, Artificial Sequence
  NFKB1 (forward primer)
cagatggcccataccttcaaat

SEQ ID NO: 46 - DNA, 22 bp, Artificial Sequence
  NFKB1 (reverse primer)
cggaaacgaaatcctctctgtt

SEQ ID NO: 47 - DNA, 21 bp, Artificial Sequence
  NRF2 (NFE2L2) (forward primer)
tccagtcagaaaccagtggat

SEQ ID NO: 48 - DNA, 21 bp, Artificial Sequence
  NRF2 (NFE2L2) (reverse primer)
gaatgtctgcgccaaaagctg

SEQ ID NO: 49 - DNA, 25 bp, Artificial Sequence
  OCT4 (forward primer)
tggagaaggagaagctggagcaaaa

SEQ ID NO: 50 - DNA, 24 bp, Artificial Sequence
  OCT4 (reverse primer)
ggcagatggtcgtttggctgaata

SEQ ID NO: 51 - DNA, 19 bp, Artificial Sequence
  PECAM1 (forward primer)
ccaaggtgggatcgtgagg

SEQ ID NO: 52 - DNA, 21 bp, Artificial Sequence
  PECAM1 (reverse primer)
tcggaaggataaaacgcggtc

SEQ ID NO: 53 - DNA, 21 bp, Artificial Sequence
  RHOA (forward primer)
tggaaagacatgcttgctcat

SEQ ID NO: 54 - DNA, 22 bp, Artificial Sequence
  RHOA (reverse primer)
gcctcaggcgatcataatcttc

SEQ ID NO: 55 - DNA, 21 bp, Artificial Sequence
  RIG1 (forward primer)
gagattttccgccttggctat

SEQ ID NO: 56 - DNA, 21 bp, Artificial Sequence
  RIG1 (reverse primer)
ccgtttcacctctgcactgtt

SEQ ID NO: 57 - DNA, 21 bp, Artificial Sequence
  SELE (forward primer)
cagcaaaggtacacacacctg

SEQ ID NO: 58 - DNA, 22 bp, Artificial Sequence
  SELE (reverse primer)
cagacccacacattgttgactt

SEQ ID NO: 59 - DNA, 19 bp, Artificial Sequence
  SELP (forward primer)
cagaccactcaaccagcag

SEQ ID NO: 60 - DNA, 19 bp, Artificial Sequence
  SELP (reverse primer)
ggccgtcagtcgagttgtc

SEQ ID NO: 61 - DNA, 25 bp, Artificial Sequence
  STAT3 (forward primer)
gggtggagaaggacatcagcggtaa

SEQ ID NO: 62 - DNA, 22 bp, Artificial Sequence
  STAT3 (reverse primer)
gccgacaatactttccgaatgc

SEQ ID NO: 63 - DNA, 19 bp, Artificial Sequence
  STAT6 (forward primer)
gttccgccacttgccaatg

SEQ ID NO: 64 - DNA, 20 bp, Artificial Sequence
  STAT6 (reverse primer)
tggatctcccctactcggtg

SEQ ID NO: 65 - DNA, 20 bp, Artificial Sequence
  STING (forward primer)
ccagagcacactctccggta

SEQ ID NO: 66 - DNA, 21 bp, Artificial Sequence
  STING (reverse primer)
cgcatttgggagggagtagta

SEQ ID NO: 67 - DNA, 21 bp, Artificial Sequence
  TIRAP (forward primer)
atggtggctttcgtcaagtca

SEQ ID NO: 68 - DNA, 23 bp, Artificial Sequence
  TIRAP (reverse primer)
tcagatactgtagctgaatcccg

SEQ ID NO: 69 - DNA, 21 bp, Artificial Sequence
  TLR9 (forward primer)
cccacctgtcactcaagtaca

SEQ ID NO: 70 - DNA, 21 bp, Artificial Sequence
  TLR9 (reverse primer)
gtggctgaaggtatcgggatg

SEQ ID NO: 71 - DNA, 20 bp, Artificial Sequence
  TP53 (forward primer)
tttgggtctttgaacccttg

SEQ ID NO: 72 - DNA, 20 bp, Artificial Sequence
  TP53 (reverse primer)
ccacaacaaaacaccagtgc

SEQ ID NO: 73 - DNA, 21 bp, Artificial Sequence
  TNFa (forward primer)
cagcctcttctccttcctgat

SEQ ID NO: 74 - DNA, 20 bp, Artificial Sequence
  TNFa (reverse primer)
gccagagggctgattagaga

SEQ ID NO: 75 - DNA, 21 bp, Artificial Sequence
  VCAM1 (forward primer)
gggaagccgatcacagtcaag

SEQ ID NO: 76 - DNA, 24 bp, Artificial Sequence
  VCAM1 (reverse primer)
aaattcggtacatcctcgacggca

SEQ ID NO: 77 - DNA, 20 bp, Artificial Sequence
  VEGFA (forward primer)
aggccagcacataggagaga

SEQ ID NO: 78 - DNA, 20 bp, Artificial Sequence
  VEGFA (reverse primer)
tttcttgcgctttcgttttt

SEQ ID NO: 79 - DNA, 18 bp, Artificial Sequence
  TBP (reference gene) (forward primer)
gcccgaaacgccgaatat

SEQ ID NO: 80 - DNA, 19 bp, Artificial Sequence
  TBP (reference gene) (reverse primer)
ccgtggttcgtggctctct

(19) United States
(12) Patent Application Publication
(10) Pub. No.: US 2016/0376650 A1
(43) Pub. Date: Dec. 29, 2016

(54) Oxidized Fraction of Extracellular DNA As A Biomarker of Stress and Methods For Using The Same

(71) Applicants: Anna BARANOVA, Annandale, VA (US); Svetlana KOSTYUK, Moscow (RU); Natalya VEIKO, Moscow (RU)

(72) Inventors: Anna BARANOVA, Annandale, VA (US); Svetlana KOSTYUK, Moscow (RU); Natalya VEIKO, Moscow (RU)

(21) Appl. No.: 15/034,956

(22) PCT Filed: Nov. 6, 2014
(86) PCT No.: PCT/US14/64331

§ 371 Date: May 6, 2016

Related U.S. Application Data
(60) Provisional application No. 61/900,705, filed on Nov. 6, 2013.

(51) Int. Cl. C12Q 1/68; C07K 16/44

(52) U.S. Cl. CPC C12Q 1/6876; C07K 16/44; C12Q 2600/112

(57) ABSTRACT

The present invention relates to methods of treating and diagnosing oxidative damage in a subject comprising administering an agent that binds oxidized extracellular nucleic acid, and methods of treating diseases and conditions in a subject comprising administering an adjuvant therapy comprising an agent that binds oxidized extracellular nucleic acid. The oxidized fraction of extracellular DNA can also be detected through electrochemical methods or by mass-spectrometry.

### FIELD OF THE INVENTION

The invention generally relates to the field of redox biology. Specifically, the invention relates to the use of the oxidized fraction of extracellular DNA in isolated bodily fluids as a biomarker for stress in the human body and methods for using the same to diagnose and treat diseases and conditions using agents, such as antibodies or fragments thereof, that bind to the oxidized fraction of extracellular DNA. The oxidized fraction of extracellular DNA can also be detected through electrochemical methods or by mass-spectrometry.

### BACKGROUND ART

Many chronic diseases are accompanied by an increase in overall oxidation of genomic DNA. Under oxidative stress, the DNA bases are prone to oxidation, with the most common products being the thymidine glycol and 8-hydroxy-2′-deoxyguanosine (8-oxodG). In fact, the 8-oxodG is the most widely used “marker” for oxidative DNA damage. The 8-oxodG is formed in DNA either via direct oxidation or can be incorporated into DNA by DNA polymerase as a modified base drawn from the nucleotide pool.

The term “cell-free circulating DNA” (cfDNA) was coined for DNA fragments that could be collected from plasma, serum, or other bodily fluids. CfDNA circulates throughout the bloodstream of both healthy people and patients with various diseases. DNA isolated from cell-free supernatants of cells cultivated in vitro is known as extracellular DNA (ecDNA). EcDNA is found in the culture medium of both intact cells and cells exposed to various types of oxidative stress.

The most widely accepted hypothesis is that the main sources of cfDNA/ecDNA are the dead cells. Another hypothesis suggests that cfDNA/ecDNA could be actively excreted into the medium by living cells [86]. Recently, cfDNA got recognition as a promising biomarker for noninvasive diagnostics and monitoring of various diseases. However, the biological role of cfDNA in normal or pathological conditions remains unclear. The functionality of these circulating DNA fragments is determined by cfDNA properties, for example, its concentration in the blood plasma and the level of oxidative modification that can be approximated by its average content of 8-oxodG.

In plasma of healthy individuals, total cfDNA concentrations vary from 1 to ˜100 ng/mL. These concentrations increase with age or in presence of various stressful conditions, for example, pregnancy, intensive exercise, or strong emotions as well as when malignancy or other chronic pathology is diagnosed. In plasma samples of patients with cancer or critical cardiovascular conditions, the concentrations of cfDNA increase up to 1000 ng/mL.

Oxidative stress is known to cause the DNA damage. The cells with the most damaged DNA die either by necrosis or by apoptosis. The oxidized DNA released from the dying cells is likely the most prominent contributor to cfDNA/ecDNA pool. Therefore, it is likely that cfDNA/ecDNA would contain larger amounts of 8-oxodG as compared to that in cellular DNA.

The cfDNA extracted from blood plasma of patients with high oxidative stress levels can significantly influence the physiological activity of intact cells. For example, when primary endotheliocytes (HUVECs) were exposed to cfDNA samples obtained from patients with hypertension and atherosclerosis, their NO contents substantially decreased, while the DNA samples obtained from healthy donors have no effect of NO release. In electrically paced cultures of ventricular neonatal rat myocytes, an exposure to the cfDNA of patients with acute myocardial infarction has produced a decrease in the frequency of contraction [108]. The cfDNA from ischemic rats decreased the levels of ROS production in neuronal cultures. Both ecDNA collected from the media of primary tumor cells cultures and cfDNA extracted from plasma of cancer patients have influenced ROS production in mesenchymal stem cells (MSCs). Importantly, cfDNAs extracted from blood of myocardial infarction and rheumatoid arthritis patients stimulate the expression of DNA sensor toll-like receptor 9 (TLR9) in MSCs, while an exposure to gDNA did not influence TLR9 levels.

An analysis of the data concerning cfDNA/ecDNA properties and the effects it produces on mammalian cells allowed us to suppose that ecDNA of irradiated cells (ecDNA^{R}) may somehow influence the other nonirradiated cells within the cell cultures thus acting as a soluble stress-signalization factor in a radiation-induced BE. Further studies confirmed this assumption, having for the first time demonstrated the significance of the bystander signaling with participation of oxidized extracellular DNA for human cells exposed to low-dose irradiation.

The main source of the ecDNA is the dead or dying cells. In a number of recent studies, it was demonstrated that ionizing low-LET irradiation increases the rate of apoptosis in various cell cultures. It seems that some subpopulations of cultured cells possess an increased sensitivity to apoptosis that may be evoked by irradiation at low doses. To pursue this hypothesis, the population of irradiation-sensitive human lymphocytes was isolated and characterized. This subpopulation was rich in large-size activated cells, could spontaneously incorporate (3H)-thymidine, had increased radiosensitivity, and decreased activity of the excision repair, as well as a high level of spontaneous chromosomal aberrations and apoptosis, all these increasing after irradiation.

The prior art data indicate that the cascade of sequential events in ecDNA-signaling may be as follows:

Irradiation→[primary oxidative stress→oxidation of gDNA→apoptosis of some portion of irradiated cells→release of oxidized ecDNA^{R}→reception of the ecDNA^{R} signal by the bystander cells→secondary oxidative stress]→oxidation of gDNA in the bystander cells→apoptosis of some portion of bystander cells→release of oxidized ecDNA, and so forth.

In this cascade, the oxidative stress propagates from irradiated cells to bystander cells (FIG. 1). The secondary oxidative stress that is evoked in intact bystander cells occurs after an interaction of the oxidized ecDNA^{R} with its receptors, or oxidized DNA sensors, that must be present on the surface or inside the bystander cells. The possible candidates for these sensors are the transmembrane proteins of the toll-like receptor family, namely, TLR9. Being transmembrane receptors, they contain a repetitive LRR domain capable of binding the ligand and a highly conservative intracellular region that ensures the interaction between the receptors and the molecules of the downstream signaling pathway, for example, an adapter protein MyD88. It is well known that the DNA fragments with unmethylated CpG motifs may serve as TLR9 ligands. In this cascade, the formation of the “DNA-TLR9” complex initiates the cellular signaling pathway that, in turn, leads to an activation of the transcription factor NF-κB, which in many different ways augments the biosynthesis of ROS. For example, TLR9 ligation may be followed by an increase in intranuclear production of NO• or O2- radical. In human monocytes, the binding of CpG-DNA to TLR9 is accompanied by secretion of both NO• and ROS, while in neutrophils it leads to the production of peroxynitrite. The slow-acting oxidants O2-, NO, and H₂O₂ are produced by sequence of metal ion-dependent enzymatic reactions that, in turn, may give rise to highly reactive compounds: OH• and hypohalogenous acids, as well as 1O₂, NO•, and NO₂•. During bystander effect, possible participation of the Fenton reaction is evidenced by the studies that showed that the radiation-induced adaptive response depends on the production of the signal molecule NO. Interestingly, in macrophages, the substitution of dG with 8-oxodG in the DNA ligand for TLR9 is accompanied by a significant increase in TNF-α cytokine. In other words, an oxidized DNA seems to be a stronger TLR9-stimulating ligand than nonoxidized DNA.

Oxidized DNA is one of the components of damage-associated molecular pattern molecules (DAMPs). Its effects can potentially increase when exposure to oxidized DNA is concomitant with the presence of other DAMPs. It might be that effects of oxidized DNA are at least in part mediated by high mobility group box 1 (HMGB1) protein whose expression is enhanced after irradiation. HMGB1 functions as an extracellular damage-associated molecular pattern molecule that promotes inflammation, cellular differentiation, survival, and migration. HMGB1 was shown as an essential component of DNA-containing complexes that stimulated cytokine production through a TLR9-MyD88 pathway. Extracellular HMGB1 accelerates the delivery of CpG-DNAs to its receptor, leading to a TLR9-dependent augmentation of IL-6, IL-12, and TNFα secretion. There is evidence that HMGB1 protein binds preferentially to damaged DNA. It was also shown that extracellular histones directly interact with TLR9 and enhance DNA-mediated TLR9 activation in immune cells.

However, no studies demonstrating that oxidized cfDNA may play a role in bystander effect in vivo have been published. Effects of exposure to oxidized cfDNA should be taken into account when treating tumors with various ROS-producing agents and irradiation. As oxidized cfDNA released from the dying tumor cells enters the circulation, it is being carried to the distant organs, with its effects expected to be systemic. For example, the damaged DNA released from irradiated cells may be responsible for abscopal effects that are suspected to be depended on actions of immune system, in particular, the ones mediated by TLRs. It is possible that artificial modulation of concentration, GC-content, and the level of oxidation of cfDNA may improve clinical outcomes in patients with various chronic diseases accompanied by extensive cell death. Accordingly, there is a need for methods that use the oxidized fraction of ecDNA/cfDNA in isolated bodily fluids as a biomarker for stress in the human body and methods for using the same to diagnose and treat diseases and conditions using agents, such as antibodies or fragments thereof, that bind to the oxidized fraction of extracellular DNA oxidized DNA.

### BRIEF SUMMARY OF THE INVENTION

In one aspect, the present invention provides a method for diagnosing the oxidative damage encountered by a subject over a recent time period, comprising the steps of obtaining a sample of blood or other biological fluid from the subject, removing all cells from the sample, extracting extracellular nucleic acid from the sample, measuring the percentage of oxidized nucleotides within the extracted extracellular nucleic acid or quantifying the total amount of oxidized nucleotides within the extracellular nucleic acid, and diagnosing the degree of oxidative damage that the subject encountered across the recent time period proportionate to the increase in the percentage of oxidized nucleotides above baseline levels, wherein baseline levels of oxidized nucleotides are calculated from either the same subject or as a per average amount of oxidized nucleotides obtained from a same-species population to which the subject belongs.

In another aspect, the invention provides a method for diagnosing the oxidative damage encountered by a subject over a recent time period, comprising the steps of attaching a wearable sensor to the body of the subject, wherein the sensor is capable of measuring the percentage of oxidized nucleotides within the extracted extracellular nucleic acid or quantifying the total amount of oxidized nucleotides within the extracellular nucleic acid over the recent time period, and diagnosing the degree of oxidative damage that the subject encountered across the recent time period proportionate to the increase in the percentage of oxidized nucleotides above baseline levels, wherein baseline levels of oxidized nucleotides are calculated from either the same subject or as a per average amount of oxidized nucleotides obtained from a same-species population to which the subject belongs, wherein the sensor provides this diagnostic information by either (i) a visual or auditory sensory signal or (ii) through a wireless signal transmitted by a wireless enabled device.

In another aspect, the invention provides a method for monitoring oxidative damage in a subject who is afflicted by a chronic disease, comprising the steps of obtaining a sample of blood or other biological fluid from the subject, removing all cells from the sample, extracting extracellular nucleic acid from the sample, measuring the percentage of oxidized nucleotides within the extracted extracellular nucleic acid or quantifying the total amount of oxidized nucleotides within the extracellular nucleic acid, and diagnosing the degree of oxidative damage that the subject accumulated over time proportionate to the increase in the percentage of oxidized nucleotides above baseline levels, wherein baseline levels of oxidized nucleotides are calculated from the same subject from an earlier period of time.

In another aspect, the invention provides a method for monitoring oxidative damage in a subject who is afflicted by a chronic disease, comprising the steps of attaching a wearable sensor to the body of the subject, wherein the sensor is capable of measuring the percentage of oxidized nucleotides within the extracted extracellular nucleic acid or quantifying the total amount of oxidized nucleotides within the extracellular nucleic acid over the recent time period, and diagnosing the degree of oxidative damage that the subject accumulated over time proportionate to the increase in the percentage of oxidized nucleotides above baseline levels, wherein baseline levels of oxidized nucleotides are calculated from either the same subject or as a per average amount of oxidized nucleotides obtained the same subject from an earlier period of time, wherein the sensor provides this diagnostic information by either (i) a visual or auditory sensory signal or (ii) through a wireless signal transmitted by a wireless enabled device.

In another aspect, the invention provides a method for monitoring aging in a subject, comprising the steps of obtaining a sample of blood or other biological fluid from the subject, removing all cells from the sample, extracting extracellular nucleic acid from the sample, measuring the percentage of oxidized nucleotides within the extracted extracellular nucleic acid or quantifying the total amount of oxidized nucleotides within the extracellular nucleic acid, and diagnosing the degree of oxidative damage that the subject accumulated over time proportionate to the increase in the percentage of oxidized nucleotides above baseline levels, wherein baseline levels of oxidized nucleotides are calculated from the same subject from an earlier period of time.

In another aspect, the invention provides a method for monitoring aging in a subject, comprising the steps of attaching a wearable sensor to the body of the subject, wherein the sensor is capable of measuring the percentage of oxidized nucleotides within the extracted extracellular nucleic acid or quantifying the total amount of oxidized nucleotides within the extracellular nucleic acid over the recent time period, and diagnosing the degree of oxidative damage that the subject accumulated over time proportionate to the increase in the percentage of oxidized nucleotides above baseline levels, wherein baseline levels of oxidized nucleotides are calculated from either the same subject or as a per average amount of oxidized nucleotides obtained the same subject from an earlier period of time, wherein the sensor provides this diagnostic information by either (i) a visual or auditory sensory signal or (ii) through a wireless signal transmitted by a wireless enabled device.

In another aspect, the invention provides a method of classifying a subject according to high or low risk of serious health complications, comprising the steps of obtaining a sample of blood or other biological fluid from the subject, removing all cells from the sample, extracting extracellular nucleic acid from the sample, measuring the percentage of oxidized nucleotides within the extracted extracellular nucleic acid or quantifying the total amount of oxidized nucleotides within the extracellular nucleic acid, and diagnosing the degree of oxidative damage that the subject encountered across a recent time period proportionate to the increase in the percentage of oxidized nucleotides above baseline levels, wherein baseline levels of oxidized nucleotides are calculated from either the same subject or as a per average amount of oxidized nucleotides obtained from a same-species population to which the subject belongs.

In another aspect, the invention provides a method of classifying a subject according to high or low risk of serious health complications, comprising the steps of attaching a wearable sensor to the body of the subject, wherein the sensor is capable of measuring the percentage of oxidized nucleotides within the extracted extracellular nucleic acid or quantifying the total amount of oxidized nucleotides within the extracellular nucleic acid over the recent time period, and diagnosing the degree of oxidative damage that the subject encountered across a recent time period proportionate to the increase in the percentage of oxidized nucleotides above baseline levels, wherein baseline levels of oxidized nucleotides are calculated from either the same subject or as a per average amount of oxidized nucleotides obtained from a same-species population to which the subject belongs, wherein the sensor provides this diagnostic information by either (i) a visual or auditory sensory signal or (ii) through a wireless signal transmitted by a wireless enabled device.

In one embodiment of the invention, the subject is human. In another embodiment, the subject is a model animal. In yet a further embodiment, the model animal is selected from the group consisting of: mouse, rat, rabbit, guinea pig, dog, cat, pig, and monkey.

In one embodiment of the invention, the subject is profiled longitudinally and the percentage of oxidized nucleotides is used for long-term monitoring of the effects of various environmental impacts. In one embodiment, the environmental pact is environmental stress. In another embodiment, the environmental stress is oxidative stress.

In one embodiment of the invention, the subject is profiled longitudinally and the percentage of oxidized nucleotides is used for long-term or short-term monitoring of the effects of cancer therapy aimed to induce tumor cell death by increasing oxidative damage in cancer cells.

In one embodiment of the invention, the percentage of oxidized nucleotides is measured chemically or electrochemically. In another embodiment, the percentage of oxidized nucleotides is measured using antibodies, aptamers, or fragments thereof. In yet another embodiment, the percentage of oxidized nucleotides is measured enzymatically.

In another aspect, the invention provides a method for evaluating the oxidative damage in a cell culture that was exposed to environmental stress, comprising the steps of removing all cells from the cell culture sample, collecting the cell-free media from the cell culture sample, extracting extracellular nucleic acid from the cell culture sample, measuring the percentage of oxidized nucleotides within the extracted extracellular nucleic acid or quantifying the total amount of oxidized nucleotides within the extracellular nucleic acid, and determining the degree of oxidative damage that the cell culture experienced as a result of exposure to the environmental stress proportionate to the increase in the percentage of oxidized nucleotides above baseline levels, wherein baseline levels of oxidized nucleotides are calculated from a similarly cultured cell line.

In one embodiment of the invention, the cell culture comprised primary cells explanted from an organism. In another embodiment, the environmental stress is a treatment with a compound with cell phenotype or gene expressing altering abilities. In another embodiment, the environmental stress is a damaging stress.

In another aspect, the invention provides a method for abating the side effects of chemotherapy in a human cancer patient, comprising removing extracellular nucleic acid from the patient's blood. In another aspect, the invention is directed to a method for abating the side effects and/or the abscopal effects of local irradiation in a human cancer patient, comprising removing extracellular nucleic acid from the patient's blood. In another aspect, the invention provides a method for abating the effects of incidental total body or partial body irradiation in a subject, comprising removing extracellular nucleic acid from the subject's blood. In one embodiment, the incidental total body or partial body irradiation occurs as a result of a nuclear accident or accidental exposure to radioactive materials. In one embodiment of the invention, extracellular nucleic acid is removed by hemosorbtion. In another embodiment, the extracellular nucleic acid is removed by plasmapheresis with a DNA-binding sorbent. In yet another embodiment, the DNA-binding sorbent is silica.

In one embodiment of the invention, the extracellular nucleic acid is extracellular DNA. In another embodiment of the invention, the oxidized nucleotide is 8-hydroxy-2′deoxyguanosine.

In another aspect, the invention provides a method of conditioning stem cells to make the cells more resistant to environmental stress, comprising the steps of expanding the cells in a cell culture medium, and adding an artificially created preparation of oxidized genomic DNA to the cells.

In another aspect, the invention provides methods of treating oxidative damage in a subject comprising administering to a subject with oxidative damage a composition comprising an agent that binds oxidized extracellular nucleic acid. In one embodiment of the invention, the subject is a human being.

In another aspect, the invention provides methods of treating a disease or condition in a subject, comprising administering to a subject with a disease or condition: a therapy suitable for treating the disease or condition and an adjuvant therapy comprising an agent that binds oxidized extracellular nucleic acid. In one embodiment of the invention, the subject is a human being.

In another aspect, the invention provides methods for diagnosing oxidative damage in a subject comprising obtaining a blood sample or fraction thereof from the subject, contacting the sample with an agent that binds oxidized extracellular nucleic acid, measuring the amount of oxidized extracellular nucleic acid in the sample relative to the amount of oxidized extracellular nucleic acid in a reference sample from a healthy subject, and diagnosing oxidative damage when measurement shows a significant elevation between the oxidized extracellular nucleic acid concentration in the sample and oxidized extracellular nucleic acid concentration in the reference sample. In one embodiment of the invention, the subject is a human being.

In one embodiment of the invention, the agent binds to one or more of modified nucleobases selected from the group consisting of: 8-hydroxyadenine, 8-hydroxy-2′-deoxyguanosine, thymine glycol, Fapy-guanine, 5-hydroxymethyl-2′-deoxyuridine, and Fapy-adenine. In another embodiment, the agent is an antibody or a fragment thereof. In another embodiment, the oxidized nucleobase or oxidized extracellular nucleic acid is measured by an electrochemical method. In another embodiment, the oxidized nucleobase or oxidized extracellular nucleic acid is measured by mass-spectrometry.

In one embodiment of the invention, the disease or condition is selected from the group consisting of: cancer, Leber's hereditary optic neuropathy, Parkinson's disease, multiple sclerosis, Alzheimer's disease, schizophrenia, chronic renal failure, Fanconi anaemia, type 1 diabetes, type II diabetes, coronary artery disease, myocardial infarction, hypertension, atherosclerosis, amyotrophic lateral sclerosis, rheumatoid arthritis, and diseases characterized by mitochondrial dysfunction. In a further embodiment of the invention, the cancer is selected from the group consisting of: breast cancer, prostate cancer, epithelial ovarian cancer, and lung cancer.

In another embodiment of the invention, the activity of NRF2 is decreased. In yet another embodiment of the invention, the activity of NF-κB is increased. In another embodiment of the invention, the activity of STAT3 is decreased.

### BRIEF DESCRIPTION OF THE DRAWINGS

FIG. 1: The proposed mechanisms for the propagation of the stress signal from irradiated cells to bystander cells. In this scheme, the 8-oxo-dG serves as a model example of DNA lesion that turns DNA fragments into the stress signal; it should be noted that other types of DNA lesions may be recognized as well. The central player that ensures amplification of the signal in this cascade is the oxidative stress. The secondary oxidative stress evoked in intact bystander cells occurs after an interaction of the oxidized ecDNA with the receptors, or oxidized DNA sensors, that must be present on the surface or inside the bystander cell. One possible candidate for oxidized DNA sensor is toll-like receptor TLR9.

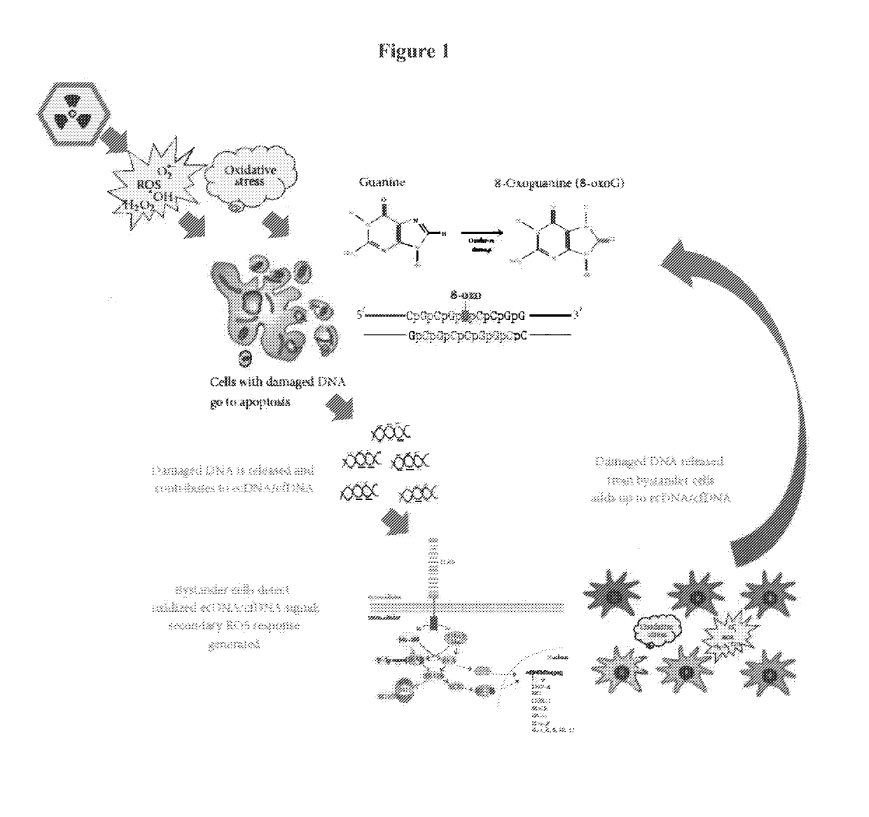

FIG. 2: Staining of MCF7 cells with various types of labeled DNA. (A) gDNA^{red}, nuclei are stained with DAPI (×40); (B) merged staining patterns of gDNA^{red} and pBR322^{green} (×200); (C) merged staining patterns of gDNA^{red-ox} and FITC-conjugated antibodies to 8-oxodG (×200); (D) FACS analysis of early endosomal marker EEA1; the distribution of the cells with varying EEA1 contents. Final concentrations of added DNA in the media were at 50 ng/mL; cells were incubated with DNA for 30 min before fixation in 3% formaldehyde. In case of staining with FITC-conjugated antibodies to 8-oxodG, fixed cells were pretreated with 0.1% Triton X100 for permeation.

FIG. 3: The exposure to gDNA^{OX} (50 ng/mL) leads to a transient increase in expression cytoplasmic DNA sensor AIM2, while not changing expression levels of TLR9. (A) intracellular localization of AIM2 (FITC-conjugated antibodies) and labeled probe gDNA^{red-ox} (×40). (B) the ratio of the levels of AIM1 [1] and TLR9 [2]-encoding RNAs to the levels TBP-encoding reference mRNA in cells exposed to gDNA or gDNA^{OX} for 2 hrs (grey columns) and 48 hrs (black columns). (C) and (D) Flow cytometry detection of AIM2 (C) and TLR9 (D) expression in MCF-7. Cells were stained with AIM2 (C) or TLR9 (D) antibody (secondary PE-conjugated antibodies). Panels C [1] and D [1]—control cells plots: FL2 versus SSC. R: gated area. Panels C [2] and D [2]: median signal intensity of FL2 (R) in MCF-7 cells (mean value for three independent experiments). Panels C [3] and D [3]: relative proportions of AIM2- or TLR9-positive cells in R gates [1]. Background fluorescence was quantified using PE-conjugated secondary antibodies. *p<0.05 against control group of cells, non-parametric U-test.

FIG. 4: The exposure to gDNA^{OX} leads to an increase in the production of ROS. (A) Microscopy-based evaluation of MCF-7 cells sequentially treated with DNA (50 ng/mL) and H2DCFH-DA (control, gDNA, gDNA^{OX} [1]) and incubated for 30 minutes (×100). Alternatively, MCF-7 cells were incubated with DNA (50 ng/mL) for 1 hour followed by addition of H2DCFH-DA and photography 30 minutes later (gDNAox [2]). (B) MCF-7 cells exposed to gDNA^{OX} (0.5 h; 50 ng/mL), were sequentially treated with Mito-tracker TMRM (15 min) and H2DCFH-DA (15 min) (×200). (C) Co-detection of labeled probe gDNA^{red} (50 ng/mL) and DCF after 30 minutes of incubation. (D) The results of the quantification of fluorescence using plate reader [1]. The time kinetics of fluorescence outputs in cells sequentially treated with H2DCFH-DA and, three minutes later, a DNA sample at final concentration of 5 or 50 ng/mL [2]. The same for cells pretreated with DNA (final concentration 5 ng/mL) for one hour, with subsequent addition of H2DCFH-DA. *) p<0.05 against control group of cells, non-parametric U-test.

FIG. 5: The analysis of 8-oxodG content in cells exposed to either gDNA or gDNAX^{OX} (50 ng/mL). (A) Cells stained with PE-labeled anti-8-oxodG antibodies and DAPI (×20). (B) Three types of anti-8-oxodG stain distribution observed in cells treated with gDNA^{OX} (×100). Cell were incubated with DNA samples for 1 hour, fixed with 3% formaldehyde, permeated with 0.1% triton X100 and stained with anti-8-oxodG (PE-conjugated secondary antibodies). (C) colocalization of 8-oxodG with mitochondria. Cells were incubated with gDNA^{OX} for 0.5 hour, Mito-tracker (30 nM, 15 min), photographed, then fixed with 3% formaldehyde, permeated with 0.1% triton X100, stained with anti-8-oxodG antibodies (FITC-conjugated secondary antibodies) and photographed again. (D) 8-oxodG content in DNA exposed cells pre-treated with NAC (FACS analysis). Cells were incubated with NAC (0.15 mM) for 30 min, then exposed to gDNAX^{OX} for 1 hour and analyzed using anti-8-oxodG antibodies (PE-conjugated secondary antibodies). Background fluorescence was quantified using PE-conjugated secondary antibodies. (E) Relative proportions of nuclei stained for 8-oxodG in non-treated control cells, cells exposed to gDNA, cells exposed to gDNA^{OX} (grey columns). Light grey column reflects cells pre-treated with NAC and exposed to gDNA^{OX}. *p<0.05 against control group of cells, non-parametric U-test.

FIG. 6: DNA damage in cells exposed to either gDNA or gDNA^{OX} at final concentration 50 ng/mL for 30 min and 2 hours. (A) comet assay in alkaline conditions [1].—Digital photography of the nuclei with varying degree of DNA damage [2,3];—cumulative histograms for tail moment and percentage of DNA within tails. The reliability of differences with the control in the obtained distributions was analyzed by means of Kolmogorov-Smirnov statistics (the table shows the values of D and α). (B) dsDNA breaks in cells exposed to gDNA^{OX} (50 ng/mL, 1 hour). Cells were processed for immunofluorescence staining with anti γH2AX antibody (×40) [1].—Three detected types of nuclei are denoted by numbers: 1—nucleus with multiple dsDNA breaks, 2—nucleus with a few dsDNA breaks, 3—nucleus with intact DNA [2].—Example of a micronucleus with dsDNA breaks. (C) FACS analysis of γ-foci A: there main fractions of the cells as evident in gating areas R1, R2, R3 [1], the distribution of γH2AX fluorescence intensities [2], relative proportions of cells within gating areas R1-R3 [3]. *p<0.05 against control group of cells, nonparametric U-test.

FIG. 7: Genome instability in MCF-7 cells exposed to gDNA^{OX} at final concentration 50 ng/mL for 24 hours. (A) multiple micronuclei [1], chromatin bridges [2], M-phase chromatin decondensation [3], non-treated control cells [4] (×100). (B) proportions of cells with micronuclei in non-treated control cells, cells exposed to gDNA, cells exposed to gDNA^{OX}. Grey columns: non-confluent, actively proliferating MCF-7 culture. Black columns: MCF-7 cells at high confluency. *p<0.05 against control group of cells, non-parametric U-test. (C) Exposure to gDNA^{OX} (50 ng/mL, 2 hours) induces formation of 8-oxodG-containing micronuclei (×100).

FIG. 8: Proliferation and cell cycle of MCF-7 cells exposed to gDNA or gDNA^{OX} at final concentration 50 ng/mL for 48 hours (FACS). A: (1)—fixed cells stained with anti-Ki-67 antibodies (green color). Background fluorescence was quantified using FITC-conjugated secondary antibodies (grey color) [2].—proportion of Ki-67-positive cells in total cell population [3].—the average signal intensity of FL1 (Ki-67+). Cells were cultivated either in absence (dark grey columns) or in presence of 0.15 mM NAC (light grey columns). B: (1) fixed cells stained with anti-PCNA antibodies (green color). Background fluorescence was quantified using FITC-conjugated secondary antibodies (grey color) [2], proportion of PCNA-positive cells in total cell population [3], the average of the median signal intensities of FL1 (PCNA+). C: (1) distribution of fluorescence intensities of the cells stained with propidium iodide. (2) distribution frequency of cells in G1-, S and G2/M phases after exposure to gDNA^{OX}. *p<0.05 against control group of cells, non-parametric U-test.

FIG. 9: Cell death in MCF-7 cultures exposed to either gDNA or gDNA^{OX} at final concentration 50 ng/mL for 48 hours. (A) Total number of cells in studied cell population. (B) (FACS)—enumeration of cells with sings of early apoptosis [1].—the distribution of fluorescence intensities of the cells stained with Annexin V-FITC (green color) FITC-conjugated secondary antibodies (grey color) [2].—control cells plots: FL1 versus SSC. R: gated area [3].—the proportion of Annexin V-positive cells in total cell population. (C) Evaluation of modified nuclei in three studies typed of MCF-7 cultures. (1)—Example of Hoechst33342 staining; (2)—Graph of the proportion of cells with modified nuclei in three studied types of MCF-7 cultures. (D) Electrophoresis [1] and evaluation of ecDNA concentrations [2] in the media of non-treated control cells and cells exposed to either gDNA or gDNAOX. Dashed line indicates amounts of ecDNA that should be present in the media when exogenous DNA is taken into account. *p<0.05 against control group of cells, non-parametric U-test.

FIG. 10: Decrease in activity of transcriptional factor NRF2 in MCF-7 cells exposed to gDNA^{OX} at final concentrations of 50 ng/mL for 2 hours. (A) FACS: the average of the median signal intensities in cells stained with anti-NRF2 antibodies after various exposures. (B) Fluorescent microscopy of cells stained to NRF2 (×40). (C) Graph of the proportion of cells with nuclear staining for NRF2 in three studied types of MCF-7 cultures. *p<0.05 against control group of cells, non-parametric U-test.

FIG. 11: Increase in activity of transcriptional factor NF-κB in MCF-7 cells exposed to gDNA^{OX} at final concentrations of 50 ng/mL for 2 hours. (A) Fluorescent microscopy of cells stained with anti-p65 (FITC) antibodies (×40). (B) Graph of the proportion of cells with nuclear staining for NF-κB in three studied types of MCF-7 cultures. (C, D) (FACS)—the average signal intensity of FL1 (p65) in cells stained with anti-p65 (C) and Ser529-phosphorylated p65 (D) antibodies [1].—distribution of fluorescence intensities of the cells stained with Ser529-phosphorylated p65 antibodies (FITC) (green color) FITC-conjugated secondary antibodies (grey color) [2].—proportion of Ser529-phosphorylated p65-positive cells in total cell population [3].—the average of the median signal intensities of FL1 (Ser529-phosphorylated p65+). Cells were cultivated either in absence (dark grey columns) or in presence of 0.15 mM NAC (light grey columns).

FIG. 12: Activity of STAT3 is stimulated in MCF-7 cells exposed to either gDNA or gDNA^{OX} at final concentrations of 50 ng/mL. (A) FACS: Frequency plot for fluorescence intensities in cells stained with anti-STAT3 antibodies [1] and the average of the median signal intensities of FL1 (STAT3) in these cells [2]. (B) Fluorescent microscopy of cells stained with STAT3 antibodies (×20) [1].—non-treated control cells and cells exposed to either gDNA or gDNA^{OX} for 2 hours [2].—cells pre-treated for 30 min by 0.15 mM NAC, then exposed to either gDNA or gDNA^{OX} for 2 hours. (C) [1]—evidence for nuclear localization of STAT3 (×100), the nuclei were stained with DAPI [2].—to evaluate the background, the cells were treated with normal rabbit IgG and FITC-conjugated secondary antibodies.

FIG. 13: A summary of events developing in MCF-7 cells exposed to oxidized DNA, and possible mediators of an adaptive response observed in these cells.

### DETAILED DESCRIPTION

In one aspect, the present invention provides a method for diagnosing the oxidative damage encountered by a subject over a recent time period, comprising the steps of obtaining a sample of blood or other biological fluid from the subject, removing all cells from the sample, extracting extracellular nucleic acid from the sample, measuring the percentage of oxidized nucleotides within the extracted extracellular nucleic acid or quantifying the total amount of oxidized nucleotides within the extracellular nucleic acid, and diagnosing the degree of oxidative damage that the subject encountered across the recent time period proportionate to the increase in the percentage of oxidized nucleotides above baseline levels, wherein baseline levels of oxidized nucleotides are calculated from either the same subject or as a per average amount of oxidized nucleotides obtained from a same-species population to which the subject belongs.

In another aspect, the invention provides a method for diagnosing the oxidative damage encountered by a subject over a recent time period, comprising the steps of attaching a wearable sensor to the body of the subject, wherein the sensor is capable of measuring the percentage of oxidized nucleotides within the extracted extracellular nucleic acid or quantifying the total amount of oxidized nucleotides within the extracellular nucleic acid over the recent time period, and diagnosing the degree of oxidative damage that the subject encountered across the recent time period proportionate to the increase in the percentage of oxidized nucleotides above baseline levels, wherein baseline levels of oxidized nucleotides are calculated from either the same subject or as a per average amount of oxidized nucleotides obtained from a same-species population to which the subject belongs, wherein the sensor provides this diagnostic information by either (i) a visual or auditory sensory signal or (ii) through a wireless signal transmitted by a wireless enabled device.

In another aspect, the invention provides a method for monitoring oxidative damage in a subject who is afflicted by a chronic disease, comprising the steps of obtaining a sample of blood or other biological fluid from the subject, removing all cells from the sample, extracting extracellular nucleic acid from the sample, measuring the percentage of oxidized nucleotides within the extracted extracellular nucleic acid or quantifying the total amount of oxidized nucleotides within the extracellular nucleic acid, and diagnosing the degree of oxidative damage that the subject accumulated over time proportionate to the increase in the percentage of oxidized nucleotides above baseline levels, wherein baseline levels of oxidized nucleotides are calculated from the same subject from an earlier period of time.

In another aspect, the invention provides a method for monitoring oxidative damage in a subject who is afflicted by a chronic disease, comprising the steps of attaching a wearable sensor to the body of the subject, wherein the sensor is capable of measuring the percentage of oxidized nucleotides within the extracted extracellular nucleic acid or quantifying the total amount of oxidized nucleotides within the extracellular nucleic acid over the recent time period, and diagnosing the degree of oxidative damage that the subject accumulated over time proportionate to the increase in the percentage of oxidized nucleotides above baseline levels, wherein baseline levels of oxidized nucleotides are calculated from either the same subject or as a per average amount of oxidized nucleotides obtained the same subject from an earlier period of time, wherein the sensor provides this diagnostic information by either (i) a visual or auditory sensory signal or (ii) through a wireless signal transmitted by a wireless enabled device.

In another aspect, the invention provides a method for monitoring aging in a subject, comprising the steps of obtaining a sample of blood or other biological fluid from the subject, removing all cells from the sample, extracting extracellular nucleic acid from the sample, measuring the percentage of oxidized nucleotides within the extracted extracellular nucleic acid or quantifying the total amount of oxidized nucleotides within the extracellular nucleic acid, and diagnosing the degree of oxidative damage that the subject accumulated over time proportionate to the increase in the percentage of oxidized nucleotides above baseline levels, wherein baseline levels of oxidized nucleotides are calculated from the same subject from an earlier period of time.

In another aspect, the invention provides a method for monitoring aging in a subject, comprising the steps of attaching a wearable sensor to the body of the subject, wherein the sensor is capable of measuring the percentage of oxidized nucleotides within the extracted extracellular nucleic acid or quantifying the total amount of oxidized nucleotides within the extracellular nucleic acid over the recent time period, and diagnosing the degree of oxidative damage that the subject accumulated over time proportionate to the increase in the percentage of oxidized nucleotides above baseline levels, wherein baseline levels of oxidized nucleotides are calculated from either the same subject or as a per average amount of oxidized nucleotides obtained the same subject from an earlier period of time, wherein the sensor provides this diagnostic information by either (i) a visual or auditory sensory signal or (ii) through a wireless signal transmitted by a wireless enabled device.

In another aspect, the invention provides a method of classifying a subject according to high or low risk of serious health complications, comprising the steps of obtaining a sample of blood or other biological fluid from the subject, removing all cells from the sample, extracting extracellular nucleic acid from the sample, measuring the percentage of oxidized nucleotides within the extracted extracellular nucleic acid or quantifying the total amount of oxidized nucleotides within the extracellular nucleic acid, and diagnosing the degree of oxidative damage that the subject encountered across a recent time period proportionate to the increase in the percentage of oxidized nucleotides above baseline levels, wherein baseline levels of oxidized nucleotides are calculated from either the same subject or as a per average amount of oxidized nucleotides obtained from a same-species population to which the subject belongs.

In another aspect, the invention provides a method of classifying a subject according to high or low risk of serious health complications, comprising the steps of attaching a wearable sensor to the body of the subject, wherein the sensor is capable of measuring the percentage of oxidized nucleotides within the extracted extracellular nucleic acid or quantifying the total amount of oxidized nucleotides within the extracellular nucleic acid over the recent time period, and diagnosing the degree of oxidative damage that the subject encountered across a recent time period proportionate to the increase in the percentage of oxidized nucleotides above baseline levels, wherein baseline levels of oxidized nucleotides are calculated from either the same subject or as a per average amount of oxidized nucleotides obtained from a same-species population to which the subject belongs, wherein the sensor provides this diagnostic information by either (i) a visual or auditory sensory signal or (ii) through a wireless signal transmitted by a wireless enabled device.

In one embodiment of the invention, the subject is human. In another embodiment, the subject is a model animal. In yet a further embodiment, the model animal is selected from the group consisting of: mouse, rat, rabbit, guinea pig, dog, cat, pig, and monkey.

In one embodiment of the invention, the subject is profiled longitudinally and the percentage of oxidized nucleotides is used for long-term monitoring of the effects of various environmental impacts. In one embodiment, the environmental pact is environmental stress. In another embodiment, the environmental stress is oxidative stress.

In one embodiment of the invention, the subject is profiled longitudinally and the percentage of oxidized nucleotides is used for long-term or short-term monitoring of the effects of cancer therapy aimed to induce tumor cell death by increasing oxidative damage in cancer cells.

In one embodiment of the invention, the percentage of oxidized nucleotides is measured chemically or electrochemically. In another embodiment, the percentage of oxidized nucleotides is measured using antibodies, aptamers, or fragments thereof. In yet another embodiment, the percentage of oxidized nucleotides is measured enzymatically.

In another aspect, the invention provides a method for evaluating the oxidative damage in a cell culture that was exposed to environmental stress, comprising the steps of removing all cells from the cell culture sample, collecting the cell-free media from the cell culture sample, extracting extracellular nucleic acid from the cell culture sample, measuring the percentage of oxidized nucleotides within the extracted extracellular nucleic acid or quantifying the total amount of oxidized nucleotides within the extracellular nucleic acid, and determining the degree of oxidative damage that the cell culture experienced as a result of exposure to the environmental stress proportionate to the increase in the percentage of oxidized nucleotides above baseline levels, wherein baseline levels of oxidized nucleotides are calculated from a similarly cultured cell line.

In one embodiment of the invention, the cell culture comprised primary cells explanted from an organism. In another embodiment, the environmental stress is a treatment with a compound with cell phenotype or gene expressing altering abilities. In another embodiment, the environmental stress is a damaging stress.

In another aspect, the invention provides a method for abating the side effects of chemotherapy in a human cancer patient, comprising removing extracellular nucleic acid from the patient's blood. In another aspect, the invention is directed to a method for abating the side effects and/or the abscopal effects of local irradiation in a human cancer patient, comprising removing extracellular nucleic acid from the patient's blood. In another aspect, the invention provides a method for abating the effects of incidental total body or partial body irradiation in a subject, comprising removing extracellular nucleic acid from the subject's blood. In one embodiment, the incidental total body or partial body irradiation occurs as a result of a nuclear accident or accidental exposure to radioactive materials. In one embodiment of the invention, extracellular nucleic acid is removed by hemosorbtion. In another embodiment, the extracellular nucleic acid is removed by plasmapheresis with a DNA-binding sorbent. In yet another embodiment, the DNA-binding sorbent is silica.

In one embodiment of the invention, the extracellular nucleic acid is extracellular DNA. In another embodiment of the invention, the oxidized nucleotide is 8-hydroxy-2′deoxyguanosine.

In another aspect, the invention provides a method of conditioning stem cells to make the cells more resistant to environmental stress, comprising the steps of expanding the cells in a cell culture medium, and adding an artificially created preparation of oxidized genomic DNA to the cells.

In another aspect, the invention provides methods of treating oxidative damage in a subject comprising administering to a subject with oxidative damage a composition comprising an agent that binds oxidized extracellular nucleic acid. In one embodiment of the invention, the subject is a human being.

In another aspect, the present invention provides methods of treating oxidative damage in a subject comprising administering to a subject with oxidative damage a composition comprising an agent that binds oxidized extracellular nucleic acid. In one embodiment of the invention, the subject is a human being.

In another aspect, the invention provides methods of treating a disease or condition in a subject, comprising administering to a subject with a disease or condition: a therapy suitable for treating the disease or condition and an adjuvant therapy comprising an agent that binds oxidized extracellular nucleic acid. In one embodiment of the invention, the subject is a human being.

In another aspect, the invention provides methods for diagnosing oxidative damage in a subject comprising obtaining a blood sample or fraction thereof from the subject, contacting the sample with an agent that binds oxidized extracellular nucleic acid, measuring the amount of oxidized extracellular nucleic acid in the sample relative to the amount of oxidized extracellular nucleic acid in a reference sample from a healthy subject, and diagnosing oxidative damage when measurement shows a significant elevation between the oxidized extracellular nucleic acid concentration in the sample and oxidized extracellular nucleic acid concentration in the reference sample. In one embodiment of the invention, the subject is a human being.

In one embodiment of the invention, the agent binds to one or more of modified nucleobases selected from the group consisting of: 8-hydroxyadenine, 8-hydroxy-2′-deoxyguanosine, thymine glycol, Fapy-guanine, 5-hydroxymethyl-2′-deoxyuridine, and Fapy-adenine. In another embodiment, the agent is an antibody or a fragment thereof. In another embodiment, the oxidized nucleobase or oxidized extracellular nucleic acid is measured by an electrochemical method. In another embodiment, the oxidized nucleobase or oxidized extracellular nucleic acid is measured by mass-spectrometry.

In one embodiment of the invention, the disease or condition is selected from the group consisting of: cancer, Leber's hereditary optic neuropathy, Parkinson's disease, multiple sclerosis, Alzheimer's disease, schizophrenia, chronic renal failure, Fanconi anaemia, type 1 diabetes, type II diabetes, coronary artery disease, myocardial infarction, hypertension, atherosclerosis, amyotrophic lateral sclerosis, rheumatoid arthritis, and diseases characterized by mitochondrial dysfunction. In a further embodiment of the invention, the cancer is selected from the group consisting of: breast cancer, prostate cancer, epithelial ovarian cancer, and lung cancer.

In another embodiment of the invention, the activity of NRF2 is decreased. In yet another embodiment of the invention, the activity of NF-κB is increased. In another embodiment of the invention, the activity of STAT3 is decreased.

### I. DEFINITIONS

To facilitate an understanding of the present invention, a number of terms and phrases are defined below.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen-binding site within the variable region of the immunoglobulin molecule. As used herein, the term encompasses intact polyclonal antibodies, intact monoclonal antibodies, single chain antibodies, antibody fragments (such as Fab, Fab′, F(ab′)2, and Fv fragments), single chain Fv (scFv) antibodies, multispecific antibodies such as bispecific antibodies, monospecific antibodies, monovalent antibodies, chimeric antibodies, humanized antibodies, human antibodies, fusion proteins comprising an antigen-binding site of an antibody, and any other modified immunoglobulin molecule comprising an antigen-binding site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1, and IgA2), based on the identity of their heavy chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises an antigen-binding site or epitope-binding site.

The term “monoclonal antibody” as used herein refers to a homogeneous antibody population involved in the highly specific recognition and binding of a single antigenic determinant or epitope. This is in contrast to polyclonal antibodies that typically include a mixture of different antibodies directed against a variety of different antigenic determinants. The term “monoclonal antibody” encompasses both intact and full-length monoclonal antibodies as well as antibody fragments (e.g., Fab, Fab′, F(ab′)2, Fv), single chain (scFv) antibodies, fusion proteins comprising an antibody portion, and any other modified immunoglobulin molecule comprising an antigen-binding site. Furthermore, “monoclonal antibody” refers to such antibodies made by any number of techniques, including but not limited to, hybridoma production, phage selection, recombinant expression, and transgenic animals.

The terms “selectively binds” or “specifically binds” mean that a binding agent or an antibody reacts or associates more frequently, more rapidly, with greater duration, with greater affinity, or with some combination of the above to the epitope, or target molecule than with alternative substances. In certain embodiments “specifically binds” means, for instance, that an antibody binds an oxidized extracellular nucleic acid with a KD of about 0.1 mM or less, but more usually less than about 1 μM. In certain embodiments, “specifically binds” means that an antibody binds a target at times with a KD of at least about 0.1 μM or less, at other times at least about 0.01 μM or less, and at other times at least about 1 nM or less. In certain alternative embodiments, an antibody may be bispecific or multispecific and comprise at least two antigen-binding sites with differing specificities. By way of non-limiting example, a bispecific agent may comprise one binding site that recognizes a modified nucleobase target and further comprise a second, different binding site that recognizes a different modified nucleobase target. Generally, but not necessarily, reference to binding means specific binding.

The terms “cancer” and “cancerous” as used herein refer to or describe the physiological condition in mammals in which a population of cells are characterized by unregulated cell growth. Examples of cancer include, but are not limited to, carcinoma, blastoma, sarcoma, and hematologic cancers such as lymphoma and leukemia.

The terms “tumor” and “neoplasm” as used herein refer to any mass of tissue that results from excessive cell growth or proliferation, either benign (non-cancerous) or malignant (cancerous) including pre-cancerous lesions.

The term “metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates (e.g., via the bloodstream or lymph) from the primary site of disease to secondary sites.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein refers to the ability of a random sample of cells from the tumor to form palpable tumors upon serial transplantation into immunocompromised hosts (e.g., mice). This definition also includes enriched and/or isolated populations of cancer stem cells that form palpable tumors upon serial transplantation into immunocompromised hosts (e.g., mice).

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The term “pharmaceutically acceptable” refers to a product or compound approved (or approvable) by a regulatory agency of the Federal government or a state government or listed in the U.S. Pharmacopeia or other generally recognized pharmacopeia for use in animals, including humans.

The terms “pharmaceutically acceptable excipient, carrier or adjuvant” or “acceptable pharmaceutical carrier” refer to an excipient, carrier or adjuvant that can be administered to a subject, together with at least one binding agent of the present disclosure, and which does not destroy the activity of the binding agent. The excipient, carrier or adjuvant should be non-toxic when administered with a binding agent in doses sufficient to deliver a therapeutic effect.

The terms “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus those in need of treatment include those already with the disorder; those prone to have the disorder: and those in whom the disorder is to be prevented. In some embodiments, a subject is successfully “treated” according to the methods of the present invention if the patient shows one or more of the following: a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

A “biomarker” is a measurable substance in an organism whose presence is indicative of some phenomenon, such as ageing, disease, infection, or environmental exposure. For example, accumulation of a biomarker over time may indicate disease progression. A biomarker as used herein is an oxidized nucleotide or oxidized nucleic acid sequence. Non-limiting examples of biomarkers include 8-hydroxyadenine, 8-hydroxy-2′-deoxyguanosine, thymine glycol, Fapy-guanine, 5-hydroxymethyl-2′-deoxyuridine, and Fapy-adenine. In one embodiment, the invention is directed to a method of diagnosing aging, disease, infection, or environmental exposure by measuring one or more biomarkers.

The term “longitudinal” pertains to a research design or survey in which the same subjects are observed repeatedly over a period of time.

In certain embodiments, the oxidized extracellular nucleic acid-binding agent comprises an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an IgA, IgD, IgE, IgG, or IgM antibody. In certain embodiments, the antibody is an IgG1 antibody. In certain embodiments, the antibody is an IgG2 antibody. In certain embodiments, the antibody is an antibody fragment comprising an antigen-binding site. In some embodiments, the antibody is a bispecific antibody. In some embodiments, the antibody is a monovalent antibody. In some embodiments, the antibody is a monospecific antibody. In some embodiments, the antibody is a multispecific antibody. In some embodiments, the antibody is conjugated to a cytotoxic moiety. In some embodiments, the antibody is isolated. In some embodiments, the antibody is substantially pure.

The binding agents of the present invention can be assayed for specific binding by any method known in the art. The immunoassays which can be used include, but are not limited to, competitive and non-competitive assay systems using techniques such as Biacore analysis. FACS analysis, immunofluorescence, immunocytochemistry, Western blot analysis, radioimmunoassay, ELISA. “sandwich” immunoassay, immunoprecipitation assay, precipitation reaction, gel diffusion precipitin reaction, immunodiffusion assay, agglutination assay, complement-fixation assay, immunoradiometric assay, fluorescent immunoassay, homogeneous time-resolved fluorescence assay (HTRF), and protein A immunoassay. Such assays are routine and well-known in the art (see, e.g., Ausubel et al., Editors, 1994-present, Current Protocols in Molecular Biology, John Wiley & Sons, Inc., New York, N.Y.).

For example, the specific binding of an agent to oxidized extracellular nucleic acid may be determined using ELISA. An ELISA assay comprises preparing antigen, coating wells of a 96 well microtiter plate with antigen, adding the binding agent conjugated to a detectable compound such as an enzymatic substrate (e.g. horseradish peroxidase or alkaline phosphatase) to the well, incubating for a period of time, and detecting the presence of the binding agent bound to the antigen. In some embodiments, the binding agent is not conjugated to a detectable compound, but instead a secondary antibody that recognizes the binding agent (e.g., an anti-Fc antibody) and is conjugated to a detectable compound is added to the well. In some embodiments, instead of coating the well with the antigen, the binding agent can be coated to the well and a secondary antibody conjugated to a detectable compound can be added following the addition of the antigen to the coated well. One of skill in the art would be knowledgeable as to the parameters that can be modified to increase the signal detected as well as other variations of ELISAs known in the art.

In certain embodiments, the oxidized extracellular nucleic acid-binding agents described herein have a circulating half-life in mice, cynomolgus monkeys, or humans of at least about 2 hours, at least about 5 hours, at least about 10 hours, at least about 24 hours, at least about 3 days, at least about 1 week, or at least about 2 weeks. In certain embodiments, the oxidized extracellular nucleic acid-binding agent is an IgG (e.g., IgG1 or IgG2) antibody that has a circulating half-life in mice, cynomolgus monkeys, or humans of at least about 2 hours, at least about 5 hours, at least about 10 hours, at least about 24 hours, at least about 3 days, at least about 1 week, or at least about 2 weeks. In certain embodiments, the oxidized extracellular nucleic acid-binding agent is an agent comprising at least one IgG (e.g., IgG1 or IgG2) constant region that has a circulating half-life in mice, cynomolgus monkeys, or humans of at least about 2 hours, at least about 5 hours, at least about 10 hours, at least about 24 hours, at least about 3 days, at least about 1 week, or at least about 2 weeks. Methods of increasing (or decreasing) the half-life of agents such as polypeptides, soluble receptors, and/or antibodies are known in the art. For example, known methods of increasing the circulating half-life of IgG antibodies include the introduction of mutations in the Fc region which increase the pH-dependent binding of the antibody to the neonatal Fc receptor (FcRn) at pH 6.0 (see, e.g., U.S. Patent Publication Nos. 2005/0276799, 2007/0148164, and 2007/0122403). Known methods of increasing the circulating half-life of antibody fragments lacking the Fc region include such techniques as PEGylation.

In some embodiments, the binding agents described herein are antibodies. Polyclonal antibodies can be prepared by any known method. In some embodiments, polyclonal antibodies are produced by immunizing an animal (e.g., a rabbit, rat, mouse, goat, or donkey) with an antigen of interest (e.g., a purified peptide fragment, full-length recombinant protein, or fusion protein) by multiple subcutaneous or intraperitoneal injections. The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from the immunized animal, usually from blood or ascites. The polyclonal antibodies can be purified from serum or ascites according to standard methods in the art including, but not limited to, affinity chromatography, ion-exchange chromatography, gel electrophoresis, and dialysis.

In some embodiments, the binding agents are monoclonal antibodies. Monoclonal antibodies can be prepared using hybridoma methods known to one of skill in the art (see e.g., Kohler and Milstein, 1975, Nature, 256:495-497). In some embodiments, using the hybridoma method, a mouse, hamster, or other appropriate host animal, is immunized as described above to elicit from lymphocytes the production of antibodies that specifically bind the immunizing antigen. In some embodiments, lymphocytes can be immunized in vitro. In some embodiments, the immunizing antigen can be a human protein or a portion thereof. In some embodiments, the immunizing antigen can be a mouse protein or a portion thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed specifically against a chosen antigen may be identified by a variety of methods including, but not limited to, immunoprecipitation, immunoblotting, and in vitro binding assays (e.g., flow cytometry, FACS, ELISA, and radioimmunoassay). The hybridomas can be propagated either in in vitro culture using standard methods (J. W. Goding, 1996, Monoclonal Antibodies: Principles and Practice, 3rd Edition, Academic Press, San Diego, Calif.) or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques as known to one skilled in the art. The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using standard techniques. The isolated polynucleotides encoding the heavy and light chains are then cloned into suitable expression vectors which produce the monoclonal antibodies when transfected into host cells such as E. coli, simian COS cells, Chinese hamster ovary (CHIO) cells, or myeloma cells that do not otherwise produce immunoglobulin proteins.

In certain other embodiments, recombinant monoclonal antibodies, or fragments thereof, can be isolated from phage display libraries expressing variable domains or CDRs of a desired species (see e.g., McCafferty et al., 1990, Nature, 348:552-554; Clackson et al., 1991, Nature, 352:624-628; and Marks et al., 1991, J. Mol. Biol., 222:581-597). In some embodiments, recombinant monoclonal antibodies, or fragments thereof, can be isolated from mammalian cell display libraries expressing variable domains or CDRs of a desired species (see e.g., U.S. patent publication No. 2011/0287979).

The polynucleotide(s) encoding a monoclonal antibody can be modified, for example, by using recombinant DNA technology to generate alternative antibodies or alternative bispecific agents. In some embodiments, the constant domains of the light and heavy chains of, for example, a mouse monoclonal antibody can be substituted for those regions of, for example, a human antibody to generate a chimeric antibody, or for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

In some embodiments, the binding agent is a humanized antibody. Typically, humanized antibodies are human immunoglobulins in which residues from the CDRs are replaced by residues from a CDR of a non-human species (e.g., mouse, rat, rabbit, hamster, etc.) that have the desired specificity, affinity, and/or binding capability using methods known to one skilled in the art. In some embodiments, the Fv framework region residues of a human immunoglobulin are replaced with the corresponding residues in an antibody from a non-human species that has the desired specificity, affinity, and/or binding capability. In some embodiments, a humanized antibody can be further modified by the substitution of additional residues either in the Fv framework region and/or within the replaced non-human residues to refine and optimize antibody specificity, affinity, and/or capability. In general, a humanized antibody will comprise substantially all of at least one, and typically two or three, variable domain regions containing all, or substantially all, of the CDRs that correspond to the non-human immunoglobulin whereas all, or substantially all, of the framework regions are those of a human immunoglobulin consensus sequence. In some embodiments, a humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject. One skilled in the art would be able to obtain a functional humanized antibody with reduced immunogenicity following known techniques (see e.g., U.S. Pat. Nos. 5,225,539; 5,585,089; 5,693,761; and 5,693,762).

In certain embodiments, the binding agent is a human antibody. Human antibodies can be directly prepared using various techniques known in the art. In some embodiments, human antibodies may be generated from immortalized human B lymphocytes immunized in vitro or from lymphocytes isolated from an immunized individual. In either case, cells that produce an antibody directed against a target antigen can be generated and isolated (see, e.g., Cole et al., 1985, Monoclonal Antibodies and Cancer Therapy. Alan R. Liss, p. 77; Boemer et al., 1991, J. Immunol., 147:86-95; and U.S. Pat. Nos. 5,750,373; 5,567,610; and 5,229,275). In some embodiments, the human antibody can be selected from a phage library, where that phage library expresses human antibodies (Vaughan et al., 1996, Nature Biotechnology, 14:309-314; Sheets et al., 1998, PNAS, 95:6157-6162; Hoogenboom and Winter, 1991, J. Mol. Biol., 227:381; Marks et al., 1991, J. Mol. Biol., 222:581). Alternatively, phage display technology can be used to produce human antibodies and antibody fragments in vitro, from immunoglobulin variable domain gene repertoires from unimmunized donors. Techniques for the generation and use of antibody phage libraries are also described in U.S. Pat. Nos. 5,969,108; 6,172,197; 5,885,793; 6,521,404; 6,544,731; 6,555,313; 6,582,915; 6,593,081; 6,300,064; 6,653,068; 6,706,484; and 7,264,963; and Rothe et al., 2008, J. Mol. Bio., 376:1182-1200. Once antibodies are identified, affinity maturation strategies known in the art, including but not limited to, chain shuffling (Marks et al., 1992, Bio/Technology, 10:779-783) and site-directed mutagenesis, may be employed to generate high affinity human antibodies.

In some embodiments, human antibodies can be made in transgenic mice that contain human immunoglobulin loci. Upon immunization these mice are capable of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

This invention also encompasses bispecific agents and bispecific antibodies. Bispecific agents are capable of specifically recognizing and binding at least two different targets or epitopes. The different targets can either be within the same molecule (e.g., two targets on a single protein) or on different molecules (e.g., one target on a protein and a second target on a second protein). In some embodiments, a bispecific agent or bispecific antibody has enhanced potency as compared to an individual agent or antibody or to a mixture of two agents. In some embodiments, a bispecific agent or bispecific antibody has reduced toxicity as compared to an individual agent or to a combination of more than one agent. It is known to those of skill in the art that any binding agent may have unique pharmacokinetics (PK) (e.g., circulating half-life). In some embodiments, a bispecific agent or bispecific antibody has the ability to synchronize the PK of two active binding agents wherein the two individual binding agents have different PK profiles. In some embodiments, a bispecific agent or bispecific antibody has the ability to concentrate the actions of two binding agents in a common area (e.g., a tumor and/or tumor environment). In some embodiments, a bispecific agent or bispecific antibody has the ability to concentrate the actions of two binding agents to a common target (e.g., a tumor or a tumor cell). In some embodiments, a bispecific agent or bispecific antibody has the ability to target the actions of two binding agents to more than one biological pathway or function.

In certain embodiments, the antibodies (or other polypeptides) described herein may be monospecific. In certain embodiments, each of the one or more antigen-binding sites that an antibody contains is capable of binding (or binds) a homologous epitope on different proteins.

In certain embodiments, the binding agent comprises an antibody fragment. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced using recombinant techniques. In some embodiments, antibody fragments include Fv or single chain Fv (scFv) fragments. Fab, Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries (Huse et al., 1989, Science, 246:1275-1281) to allow rapid and effective identification of monoclonal Fab fragments with the desired specificity for oxidized extracellular nucleic acid. In some embodiments, antibody fragments are linear antibody fragments. In certain embodiments, antibody fragments are monospecific or bispecific. In certain embodiments, the binding agent is a scFv. Various techniques can be used for the production of single-chain antibodies specific to oxidized extracellular nucleic acid.

In some embodiments of the present invention, the oxidized extracellular nucleic acid-binding agents are polypeptides. The polypeptides can be recombinant polypeptides, natural polypeptides, or synthetic polypeptides comprising an antibody, or fragment thereof, that bind oxidized extracellular nucleic acid. It will be recognized in the art that some amino acid sequences of the binding agents described herein can be varied without significant effect on the structure or function of the protein. Thus, the invention further includes variations of the polypeptides which show substantial activity or which include regions of an antibody, or fragment thereof, against oxidized extracellular nucleic acid. In some embodiments, amino acid sequence variations of oxidized extracellular nucleic acid-binding polypeptides include deletions, insertions, inversions, repeats, and/or other types of substitutions.

In some embodiments, the polypeptides described herein are isolated. In some embodiments, the polypeptides described herein are substantially pure.

The polypeptides, analogs and variants thereof, can be further modified to contain additional chemical moieties not normally part of the polypeptide. The derivatized moieties can improve or otherwise modulate the solubility, the biological half-life, and/or absorption of the polypeptide. The moieties can also reduce or eliminate undesirable side effects of the polypeptides and variants. An overview for chemical moieties can be found in Remington: The Science and Practice of Pharmacy, 22st Edition, 2012, Pharmaceutical Press, London.

The polypeptides described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g., Zoeller et al., 1984, PNAS, 81:5662-5066 and U.S. Pat. No. 4,588,585.

In other embodiments, oxidized extracellular nucleic acid can be detected by other methods, e.g., electrochemical detection or by mass-spectrometry. Oxidized extracellular nucleic acid can be measured by conventional mass-spectrometry (MS) or GC-MS methods. Oxidized extracellular nucleic acid can also be detected using methods currently embedded in nucleic acid sequencing machines. For example, Clark, T. A. et al., Genome Integrity 2:10 (2011) describes direct detection and sequencing of damaged DNA bases using the Single Molecule, Real-Time (SMRT®) Sequencing platform of Pacific Biosciences® on the PacBio RS sequencing system. Other commercially available sequencers include the ABI sequencer, Hiseq 2000, Hiscan Sequencers, MiSeq sequencers, and Ion Torrent PGM sequencers.

### II. METHODS OF USE AND PHARMACEUTICAL COMPOSITIONS

The present invention provides methods of treating cancer in a subject (e.g., a subject in need of treatment) comprising administering a therapeutically effective amount of an oxidized extracellular nucleic acid-binding agent described herein to the subject. In certain embodiments, the subject is a human. In certain embodiments, the subject has a cancerous tumor. In certain embodiments, the subject has had a tumor removed. The invention also provides a bispecific agent or antibody for use in a method of treating cancer, wherein the bispecific agent or antibody is an agent or antibody described herein. The invention also provides the use of a bispecific agent or antibody described herein for the manufacture of a medicament for the treatment of cancer.

In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, bladder cancer, glioblastoma, and head and neck cancer. In certain embodiments, the cancer is ovarian cancer. In certain embodiments, the cancer is colorectal cancer or colon cancer. In certain embodiments, the cancer is pancreatic cancer. In certain embodiments, the cancer is breast cancer, including triple negative breast cancer. In certain embodiments, the cancer is prostate cancer. In certain embodiments, the cancer is lung cancer, including non-small cell lung cancer and small cell lung cancer.

In some embodiments, the subject's cancer/tumor may be refractory to certain treatment(s). As a non-limiting example, the subject's cancer (or tumor) may be chemorefractory. In some embodiments, the subject's cancer may be resistant to EGFR inhibitors.

Methods of treating a disease or disorder in a subject, wherein the disease or disorder is characterized by an increased level of stem cells and/or progenitor cells are further provided. In some embodiments, the treatment methods comprise administering a therapeutically effective amount of an oxidized extracellular nucleic acid-binding agent, polypeptide, or antibody described herein to the subject.

The present invention provides methods of selecting a human subject for treatment with an oxidized extracellular nucleic acid-binding agent, comprising determining if the subject has an elevated fraction of oxidized extracellular nucleic acid. In some embodiments, the “elevated” or “high” level of oxidized extracellular nucleic acid is in comparison to the level of the fraction of oxidized extracellular nucleic acid in the same tissue type of healthy subjects. In some embodiments, the “elevated” or “high” level of oxidized extracellular nucleic acid is in comparison to the level in a reference sample. In some embodiments, if selected for treatment, the subject is administered an oxidized extracellular nucleic acid-binding agent described herein. In some embodiments, the oxidized extracellular nucleic acid-binding agent is an anti-modified nucleobase antibody. In some embodiments, the antibody binds to 8-hydroxy-2′-deoxyguanosine. In some embodiments, the oxidized extracellular nucleic acid-binding agent is a bispecific agent.

The present invention also provides methods of treating cancer in a human subject, comprising: (a) selecting a subject for treatment based, at least in part, on the subject having a cancer that has an elevated or high fraction of oxidized extracellular nucleic acid, and (b) administering to the subject a therapeutically effective amount of an oxidized extracellular nucleic acid-binding agent described herein as an adjuvant therapy.

Methods for determining whether a tumor or cancer has an elevated or high level of oxidized extracellular nucleic acid can use a variety of samples. In some embodiments, the sample is taken from a subject having a tumor or cancer. In some embodiments, the sample is a fresh whole blood sample. In some embodiments, the sample is a frozen whole blood sample. In some embodiments, the sample is a plasma sample. In some embodiments, the sample is a serum sample. In some embodiments, the sample is processed to extracellular DNA.

The present invention further provides pharmaceutical compositions comprising the binding agents described herein. In certain embodiments, the pharmaceutical compositions further comprise a pharmaceutically acceptable vehicle. These pharmaceutical compositions find use in inhibiting tumor growth and/or treating cancer in a subject (e.g., a human patient).

In certain embodiments, formulations are prepared for storage and use by combining an agent of the present invention with a pharmaceutically acceptable vehicle (e.g., a carrier or excipient). Suitable pharmaceutically acceptable vehicles include, but are not limited to, non-toxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22st Edition, 2012, Pharmaceutical Press, London).

The pharmaceutical compositions of the present invention can be administered in any number of ways for either local or systemic treatment. Administration can be topical by epidermal or transdermal patches, ointments, lotions, creams, gels, drops, suppositories, sprays, liquids, and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

### III. KITS COMPRISING OXIDIZED EXTRACELLULAR NUCLEIC ACID-BINDING AGENTS

The present invention provides kits that comprise the oxidized extracellular nucleic acid-binding agents (e.g., antibodies or bispecific agents) described herein and that can be used to perform the methods described herein. In certain embodiments, a kit comprises at least one purified antibody against oxidized extracellular nucleic acid or at least one purified bispecific agent that binds oxidized extracellular nucleic acid and one or more additional therapeutic agents. In certain embodiments, the second (or more) therapeutic agent is a chemotherapeutic agent. In certain embodiments, the second (or more) therapeutic agent is an angiogenesis inhibitor.

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, may be practiced without departing from the scope of the present disclosure.

### EXAMPLES

### Example 1

### Cell Culture

ER/PR-positive MCF-7 breast cancer cells were purchased at ATCC, Manassas, USA (Cat: HTB-22). Human embryonic lung fibroblasts were retrieved from the biospecimen collection maintained by the Research Centre for Medical Genetics, Russian Academy of Medical Sciences collection and grown as described in [7]. Ethical approval for the use of primary human cells was obtained from the Committee for Medical and Health Research Ethics of Research Centre for Medical Genetics, Russian Academy of Medical Sciences (2012, approval number 5).

MCF-7 cells were cultured in DMEM medium supplemented with 10% (v/v) fetal calf serum, 2 mM L-glutamine, 100 units/mL penicillin, and 100 μg/mL of streptomycin. Cells were grown in a humidified atmosphere with 5% CO₂ in air at 37° C. Before treatment with DNA probes, cells were grown for 24 h or 72 h in slide flasks.

### Example 2

### Flow Cytometry

Before flow cytometry, cells were washed in Versene solution, than treated with 0.25% trypsin under control of light microscopic observation. Cells were transferred to the Eppendorf tubes, washed with culture media, then centrifuge and resuspended in PBS. Staining of the cells with various antibodies was performed as described below. Briefly, to fix the cells, the paraformaldehyde (Sigma) was added at a final concentration of 2% at 37° C. for 10 min. Cells were washed three times with 0.5% BSA-PBS and permeabilized with 0.1% Triton X-100 (Sigma) in PBS for 15 min or with 70% ethanol at 4° C. Cells (˜50×10³) were washed three times with 0.5% BSA-PBS and stained with 1-2 μg/mL FITC-γH2AX (Ser139) antibody (Temecula Calif.), FITC-Ki-67 antibody, PCNA, 8-oxodG, EEA1, AIM2, TLR9, NRF2, NF-κB (p65), S529 NF-κB (p65) and STAT3 antibodies (Abcam) for 3 h at 4° C., then again washed thrice with 0.5% BSA-PBS and stained with 1 μg/mL secondary FITC-conjugated or PE-conjugated antibodies (Abcam) for 1 h at 4° C. To quantify intracellular DNA, cells were treated with propidium iodide and RNAase A. To quantify the background fluorescence, a portion of the cells were stained with secondary FITC(PE)-conjugated antibodies only. Cells were analyzed at CyFlow Space (Partec, Germany).

### Annexin V Binding Assays.

Following treatment with gDNA or gDNA^{OX}, cells were detached by trypsinization, counted and pelleted (1000 r.p.m. for 5 min). Cell pellets were washed once with PBS and once in Annexin V binding buffer (10 mM HEPES, pH 7.4, 140 mM NaCl, 2.5 mM CaCl₂). Cells were treated with Annexin V-FITC at room temperature for 15 min in the dark. Cells were analyzed for fluorescence on CyFlow Space.

### Example 3

### Fluorescent Microscopy

Cell images were obtained using the AxioScope A1 microscope (Carl Zeiss).

### Immunocytochemistry.

MCF-7 cells were fixed in 3% formaldehyde (4° C.) for 20 min, washed with PBS and then permeabilized with 0.1% Triton X-100 in PBS for 15 min at room temperature, followed by blocking with 0.5% BSA in PBS for 1 h and incubated overnight at 4° C. with the FITC-γH2AX (Ser139), 8-oxodG, NRF2, STAT3, NF-κB (p65), AIM2 antibody. After washing with 0.01% Triton X-100 in PBS MCF-7 cells were incubated for 2 h at room temperature with the FITC/PE goat anti-mouse IgG, washed with PBS and then stained with DAPI.

### Intracellular Localization of Labeled DNA Fragments.

Labeled fractions of gDNA^{Red}, gDNA^{Red-OX} and pBR322^{Green} (50 ng/ml) were added to cultivation media for 30 min. Cells were washed three times with PBS, fixed in 3% paraformaldehyde (4° C.) for 20 min, washed with PBS and stained with 2 μg/mL DAPI. To analyze distribution of 8-oxodG, MCF-7 cells were permeabilized with 0.1% Triton X-100 in PBS for 15 min at room temperature, then treated with respective antibodies.

### Analysis of Genomic Instability.

Before treatment with DNA probes, cells were grown for 24 h or 72 h in slide flasks. The DNA fractions were added to cultivation media for 24 hours. Cells were fixed in 3% formaldehyde (4° C.) for 20 min, washed with PBS and stained with 2 μg/mL DAPI. Approximately 2,000 cells were investigated for the presence of micronuclei, nuclear buds and nuclear bridges as described by Fenech (2009).

### Nuclear Fragmentation.

Was examined by Hoechst 33342 (Sigma) staining (10 μg/mL) for 10 min at 37° C. 1,000 cells were investigated for the presence of the damaged nuclei.

### ROS Detection Assays.

Cells were grown in slide flasks and treated in two different protocols [1]. MCF-7 cell cultures were pretreated with 5 μM of H2DCFH-DA (Molecular Probes/Invitrogen, CA, USA) for 5 min, then ecDNA samples were added for further 30 min; (2) ecDNA samples were added to MCF-7 cultures, cell were grown for 1 hour, then cells were treated with 5 μM of H2DCFH-DA for 30 min. In both cases, cells were washed three times with PBS and immediately photographed.

### Mitochondria.

In cells were stained with 30 nM TMRM (tetramethylrhodamine methyl ester) (Molecular Probes) for 20 min at 37° C.

### Example 4

### Extraction of the DNA Fragments from the Cells or the Cell-Free Media

To extract extracellular DNA, cells were removed from the media by centrifugation at 460×g, followed by mixing of 3 mL of the media with 0.3 mL of the solution containing 1% sodium lauryl sarcosylate, 0.02 M EDTA, and 75 μg/mL RNAse A (Sigma, USA), incubation for 45 min, then the 24-h treatment with proteinase K (200 μg/mL, Promega, USA) at 37° C. Intact gDNA was extracted from primary human embryonic fibroblasts (HEFs) [7]. To extract genomic DNA, cells separated, and the DNA was extracted form lysed cells. After two cycles of the purification with saturated phenolic solution, DNA fragments were precipitated by adding two volumes of ethanol in the presence of 2M ammonium acetate. The precipitate was then washed with 75% ethanol twice, then dried and dissolved in water. The concentration of DNA was determined by measuring fluorescence intensity after DNA staining with the RiboGreen (Molecular Probes/Invitrogen, CA, USA). Mean size of untreated gDNA fragments was 30 kb. To match gDNA and gDNA^{OX} samples in its mean size, gDNA was hydrolyzed by DNAse I until size distribution of its fragments became from 0.2 to 15 kb.

### Example 5

### Generation of the DNA Samples

gDNA^{OX}.

gDNA solution (100 ng/mL) was combined with H₂O₂(300 mM) under UV light (312 nm) for 30 min, 25° C. [15]. Modified DNA was precipitated with 2 volumes of ethanol in the presence of 2 M ammonium acetate, then washed twice with 75% ethanol, dried and dissolved in water. Resulting DNA concentrations were assessed by the analysis of the UV spectra. The size distribution of its gDNA^{OX} fragments was from 0.2 to 15 kb.

gDNA^{red} and pBR322^{green}.

Labeling of extracted genomic and plasmid DNA was performed by nick translation using CGH Nick Translation Kit (Abbott Molecular) under manufacturer's protocol with slight modification. Solutions of genomic human and plasmid DNA (3 μg/μL) were labeled with SpectrumRed and SpectrumGreen, respectively. In the reaction mix, 50% of the dTTP was substituted with the labeled dUTP. About 20% of the fluorescent-labeled nucleotide was incorporated into the DNA, while unincorporated nucleotides were removed by ethanol precipitation. The fragment size was in 300-3000 bp range as determined by electrophoresis in 1% agarose.

gDNA^{red-OX}.

gDNA^{red} (100 ng/ml) and gDNA^{OX} (100 ng/ml) were heated to 75° C. in 70% formamide-PBS and slowly cooled to 42° C. using the StepOne Plus (Applied Biosystems), then kept at 37° C. for a few hours.

### Example 6

### Quantification of mRNA Levels

Total mRNA was isolated from cells using RNeasy Mini kit (Qiagen, Germany). After the treatment with DNAse I, RNA samples were reverse transcribed by Reverse Transcriptase kit (Sileks, Russia). The expression profiles were obtained using quantitative reverse transcriptase polymerase chain reaction (qRT-PCR) with SYBRgreen PCR MasterMix (Applied Biosystems). Three housekeeping genes, ACTB, GADPH and TBP, were evaluated as possible reference genes in MCF-7 exposed to oxidized DNA. An expression of TBP was found the most stable and the employed as reference standard in further experiments. The mRNA levels were analyzed in several independent experiments using the StepOne Plus (Applied Biosystems); the technical error (% CV) was approximately 2%. All PCR products were run in the polyacrylamide gel (PAGE) to confirm their size. The following primers were used (Sintol, Russia):

AIM2
(F: CAGAAATGATGTCGCAAAGCAA,
R: TCAGTACCATAACTGGCAAACAG)
BCL2
(F: GCCTTCTTTGAGTTCGGTGG,
R: ATCTCCCGGTTTGACGCTCT)
BCL2A1 (Bfl-1/A1)
(F: TACAGGCTGGCTCAGGACTAT,
R: CGCAACATTTTGTAGCACTCTG)
BCL2L1 (BCL-X)
(F: CGACGAGTTTGAACTGCGGTA,
R: GGGATGTCAGGTCACTGAATG)
BIRC3 (c-IAP1)
(F: AAGCTACCTCTCAGCCTACTTT,
R: CCACTGTTTTCTGTACCCGGA)
BMP2
(F: ACTACCAGAAACGAGTGGGAA,
R: CATCTGTTCTCGGAAAACCTGAA)
BMP4
(F: AAAGTCGCCGAGATTCAGGG,
R: GACGGCACTCTTGCTAGGC)
BRCA1
(F: TGTGAGGCACCTGTGGTGA,
R: CAGCTCCTGGCACTGGTAGAG)
CDKN2A (p16INK4)
(F: ATGGAGCCTTCGGCTGACT,
R: TAACTATTCGGTGCGTTGGG)
CDKN1A (p21CIP1/WAF1)
(F: GGAAGACCATGTGGACCTGT,
R: ATGCCCAGCACTCTTAGGAA)
FOS
(F: GGGGCAAGGTGGAACAGTTAT,
R: CCGCTTGGAGTGTATCAGTCA)
GATA-4
(F: GCCCAAGAACCTGAATAAATCTAAG,
R: AGACATCGCACTGACTGAGAACGTC)
ICAM1
(F: CGTGCCGCACTGAACTGGAC,
R: CCTCACACTTCACTGTCACCT)
IL10
(F: AAGGCGCATGTGAACTCCC,
R: ACGGCCTTGCTCTTGTTTTC)
IL6
(F: AAATTCGGTACATCCTCGACGGCA,
R: AGTGCCTCTTTGCTGCTTTCACAC)
IL8
(F: ACTGAGAGTGATTGAGAGTGGAC,
R: AACCCTCTGCACCCAGTTTTC)
JUN
(F: TCCAAGTGCCGAAAAAGGAAG,
R: CGAGTTCTGAGCTTTCAAGGT)
KEAP1
(F: GTGGTGTCCATTGAGGGTATCC,
R: GCTCAGCGAAGTTGGCGAT)
MAP4K4
(F: GAGCCACAGGTACAGTGGTC,
R: AAGCCTTTTGGGTAGGGTCAG)
MAPK8 (JNK1)
(F: AGAAGCTAAGCCGACCATTTC,
R: TCTAGGGATTTCTGTGGTGTGA)
MYD88
(F: GGCTGCTCTCAACATGCGA,
R: TGTCCGCACGTTCAAGAACA);
NANOG
(F: GCTGAGATGCCTCACACGGAG,
R: TCTGTTTCTTGACTGGGACCTTGTC);
NFKB1
(F: CAGATGGCCCATACCTTCAAAT,
R: CGGAAACGAAATCCTCTCTGTT);
NRF2 (NFE2L2)
(F: TCCAGTCAGAAACCAGTGGAT,
R: GAATGTCTGCGCCAAAAGCTG);
OCT4
(F: TGGAGAAGGAGAAGCTGGAGCAAAA,
R: GGCAGATGGTCGTTTGGCTGAATA);
PECAM1
(F: CCAAGGTGGGATCGTGAGG,
R: TCGGAAGGATAAAACGCGGTC);
RHOA
(F: TGGAAAGACATGCTTGCTCAT,
R: GCCTCAGGCGATCATAATCTTC);
RIG1
(F: GAGATTTTCCGCCTTGGCTAT,
R: CCGTTTCACCTCTGCACTGTT);
SELE
(F: CAGCAAAGGTACACACACCTG,
R: CAGACCCACACATTGTTGACTT);
SELP
(F: CAGACCACTCAACCAGCAG,
R: GGCCGTCAGTCGAGTTGTC);
STAT3
(F: GGGTGGAGAAGGACATCAGCGGTAA,
R: GCCGACAATACTTTCCGAATGC);
STAT6
(F: GTTCCGCCACTTGCCAATG,
R: TGGATCTCCCCTACTCGGTG);
STING
(F: CCAGAGCACACTCTCCGGTA,
R: CGCATTTGGGAGGGAGTAGTA);
TIRAP
(F: ATGGTGGCTTTCGTCAAGTCA,
R: TCAGATACTGTAGCTGAATCCCG);
TLR9
(F: CCCACCTGTCACTCAAGTACA,
R: GTGGCTGAAGGTATCGGGATG);
TP53
(F: TTTGGGTCTTTGAACCCTTG,
R: CCACAACAAAACACCAGTGC);
TNFα
(F: CAGCCTCTTCTCCTTCCTGAT,
R: GCCAGAGGGCTGATTAGAGA);
VCMA1
(F: GGGAAGCCGATCACAGTCAAG,
R: AAATTCGGTACATCCTCGACGGCA);
VEGFA
(F: AGGCCAGCACATAGGAGAGA,
R: TTTCTTGCGCTTTCGTTTTT);
TBP (reference gene)
(F: GCCCGAAACGCCGAATAT,
R: CCGTGGTTCGTGGCTCTCT).

### Example 7

### Blocking ROS

Some experiments were supplemented with controls exposed to both oxidized DNA and antioxidant N-acetylcysteine (NAC) at 0.15 mM. In these cases, NAC was added to the media 30 minutes before exposure to DNA.

### Example 8

### Statistics

All reported results were reproduced at least three times as independent biological replicates. In FACS, the mean values of signal intensities were analyzed. The Figures show the average data and the standard deviation (SI)). The significance of the observed differences was analyzed using non-parametric Mann-Whitney U-tests. P-values <0.05 were considered statistically significant H marked at Figures with (*). Data were analyzed with StatPlus2007 Professional software (http://www.analystsoft.com).

### Example 9

### Localization of gDNA and gDNAOX in MCF-7 Cells

Concentrations of ecDNA in the media conditioned by intact MCF-7 cells were, on average, at 140±20 ng/mL. Effects of gDNA and gDNA^{OX} were evaluated after adding various concentrations of respective DNA to the cultivation media. Intact gDNA was extracted from primary human embryonic fibroblasts (HEFs), while gDNA^{OX} samples were obtained as a result of the treatment of gDNA with H₂O₂ as described before [15]. Levels of 8-oxodG in gDNA were at ˜0.1 8-oxodG per one million of 2′-deoxynucleosides, while in gDNA^{OX} these levels were at ˜750 8-oxodG per one million of 2′-deoxynucleosides [5,7]. To ensure that gDNA matches gDNA^{OX} by mean length of its fragments and their size distribution (0.2 to 15 kb), gDNA was treated with various concentrations of DNAse I and the matching gDNA sample was selected after electrophoretic evaluation in agarose gels. Comparative effects of gDNA and gDNA^{OX} treatments were studied at final media concentrations of 50 ng/mL or 5 ng/mL, while exposure varied from 30 minutes to 48 hours.

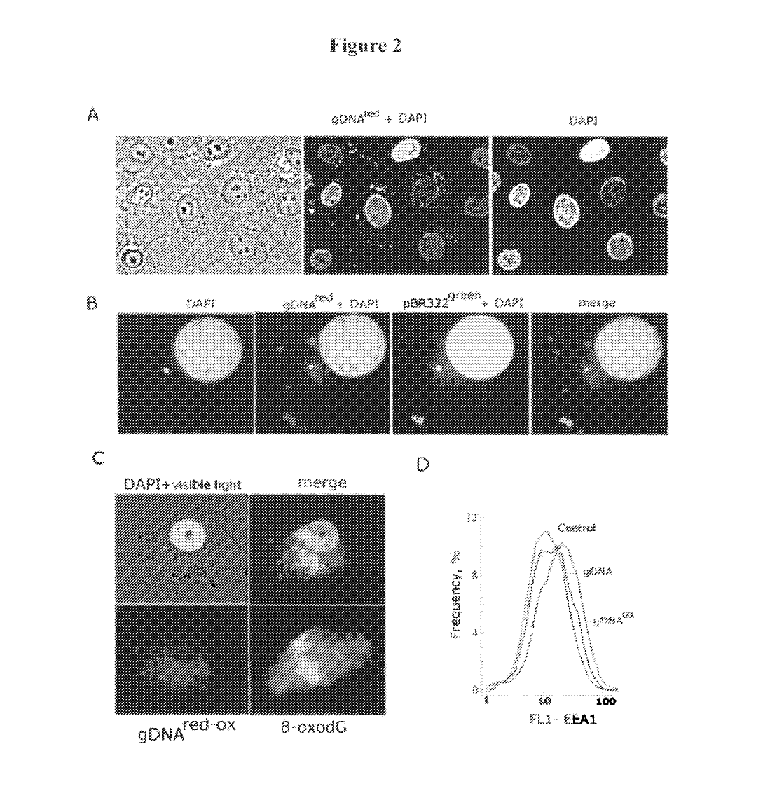

To find out the intracellular locations of gDNA and gDNA^{OX}, a number of DNA probes were synthesized and differentially labeled. gDNA^{red} and pBR322^{green} probes were labeled using nick-translation with SpectrumRed and SpectrumGreen, respectively. In MCF-7 cells, gDNA^{red} and pBR322^{green} demonstrate similar granulated, clumped staining patterns in the periphery of the cytoplasm, visible in approximately 70% of cells (FIG. 2A). More detailed analysis showed that intracellular distribution of labeled DNA fragments is sample specific (FIG. 2B). In cells treated with both gDNA^{red} and pBR322green, some areas of the cytoplasm are stained with one, but not the other type of labeled DNA. Area stained with more sequence-diverse gDNA^{red} are present in larger numbers and occupy a larger volume of the cell. In gDNA^{red} stained cells there was also a diffuse staining near the nuclear envelope that was visible at a higher magnification (×200). Based on observations, at least some exogenous gDNA fragments are imported into the cell.

To determine the intracellular locations for gDNA^{OX}, a composite probe was produced by slow renaturation of nick-translation labeled gDNA^{red} and gDNA^{OX} (gDNA^{red-OX}). Similar to gDNA^{red}, this composite labeled probe was also located at the periphery of the cytoplasm (FIG. 2C), however, in case of the composite probe gDNA^{red-OX}, a substantial portion of the labeled fragments were found inside of the cytoplasm near the nucleus. To confirm that this diffuse staining corresponded to oxidized DNA, the cells were stained with FITC-conjugated antibodies to 8-oxodG (FIG. 2). The data indicated that gDNA^{OX} is imported into the cell at a substantially larger degree than gDNA. After entering the cell, gDNA^{OX} locates in the cytoplasm, forming foci around the nucleus.

Endocytosis is one of the common ways of delivery of exogenous compounds into the cell. The formation of novel endosomes is accompanied by an increase in expression of early endosome antigen 1 protein (EEA1), known as an early endosomal biomarker [26]. Using FACS, it was demonstrated that exposure to DNA^{OX} leads to an increase of the proportion of cells that express high levels of EEA1 (FIG. 2D). These observations are in concert with visual patterns of intracellular staining for gDNA^{OX}.

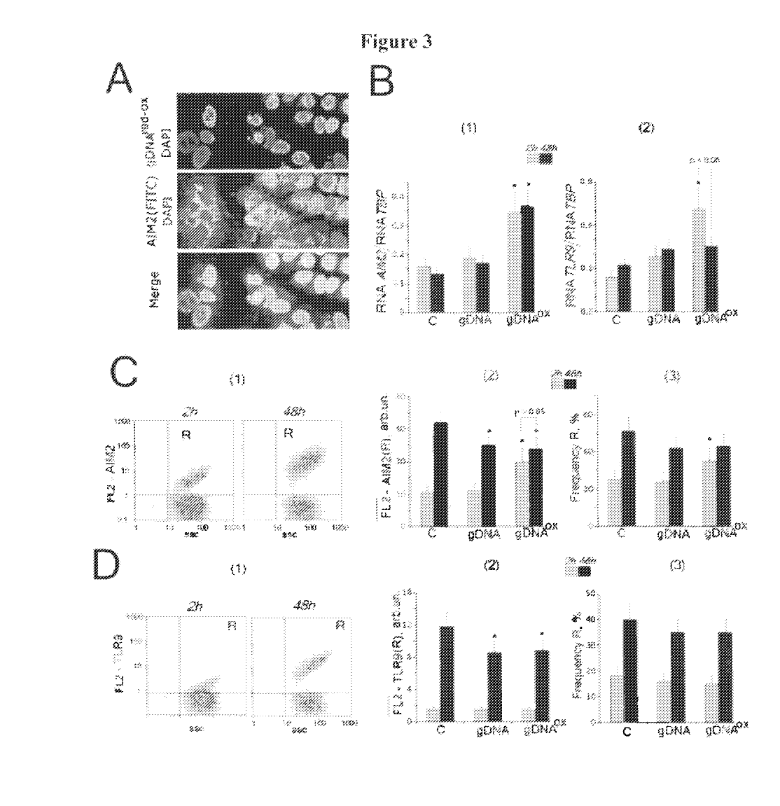

It is known that intracellular sensors are capable of binding to DNA fragments either inside the cytoplasm (AIM2, RIG1, STING) [27] or within the endosomes (TLR9) [28]. Interestingly, 2-hours exposure to gDNA^{OX} stimulates the expression of mRNAs encoding AIM2, TLR9 and RIG1 (Table 1). Two DNA sensors, AIM2 and TLR9, were studied in greater details (FIG. 3).

### AIM2

In non-confluent MCF-7 cells, the levels of AIM2 mRNA (FIG. 3B [1]) and protein expression (FIG. 3C) are low. In control cells, the protein levels of AIM2 correlate with the degree of confluency. In non-confluent cultures, AIM2 is expressed in about 25% of cells (FIG. 3C [1,3]). In confluent cultures, the proportion of cells with AIM2 increases 2-fold (FIG. 3C[1,3]). These increases are paralleled by increases in AIM2 protein levels per cell (FIG. 3C[2]), while the levels of AIM2 encoding mRNAs remain approximately the same (FIG. 3B[1]). These observations may be explained by prevailing regulation of AIM2 activity at the level of the translation or its stability rather than at the level of transcription and await further investigation.

Merged staining patterns for FITC-conjugated anti-AIM2 antibodies and labeled probe gDNA^{red-ox} are shown in FIG. 3A. Many stained areas, indeed, overlap, possibly indicating an interaction between gDNA^{OX} with AIM2 sensors. In cultured MCF-7 cells exposed to oxidized DNA, the levels of both AIM2 protein and its mRNA are elevated (FIGS. 3B[1] and 3C). In AIM2-positive population of cells, an exposure to either oxidized DNA or genome DNA for 48 hours leads to the drop in the levels of AIM2 protein per cell (FIG. 3C[2]).

### TLR9

In non-confluent MCF-7 cells, the levels of TLR9 are low, with approximately 20% of cells stained (FIG. 3B[2], D), in agreement with previous studies [28]. In confluent MCF-7 cultures, the proportion of cells expressing TLR9 protein increases to approximately 40% (FIG. 3D[3]) along with the intensities of TLR9 staining of individual cells (FIG. 3D[2]). Similarly to the levels of AIM2 encodings mRNAs, the levels of TLR9 encodings mRNAs remain unchanged (FIG. 3B[2]). After 2 hours of exposure to oxidized DNA, the levels of TLR9, encoding mRNA increase, while amounts of TLR9 protein in individual cells do not change.

TABLE 1
The changes in expression levels of select mRNAs
after exposure of MCF-7 cells to either gDNA or gDNA

[symbol]

| | cDNA, 60 ng/mL | | dDNA [symbol] , 60 ng [symbol] | |
|---|---|---|---|---|
| symbol gene | 2 h | 48 h | 2 h | 48 h |

Cell Cycle [symbol] and Cell Cycle

| | | | | |
|---|---|---|---|---|
| CDKN2A [symbol] ) | 1.8 ± 0.5 | 3.3 ± 0.3 [symbol] | 1.6 ± 0.1 [symbol] | 2.5 ± 0.3 [symbol] |
| CDKN1A (p21C1P1/WAFT) | 1.3 ± 0.3 | 2.9 ± 0.2 [symbol] | 1.1 ± 0.2 | 2.2 ± 0.2 [symbol] |
| TP53 | 0.6 ± 0.0 | 1.6 ± 0.2 [symbol] | 2.6 ± 0.3 [symbol] | 0.1 ± 0.2 [symbol] |

Anti-Apoptotic

| | | | | |
|---|---|---|---|---|
| BCL2 | 1.2 ± 0.2 | 2.6 ± 0.3 [symbol] | 3.3 ± 0.3 [symbol] | 3.2 ± 0.2 [symbol] |
| BCL2A1 ( [symbol] -1(A1) | 1.1 ± 0.3 | 2.0 ± 0.2 [symbol] | 5.0 ± 0.3 [symbol] | 1.8 ± 0.3 [symbol] |
| BCL2L1 (Bct-X) | 1.0 ± 0.2 | 1.9 ± 0.3 [symbol] | 1.2 ± 0.3 | 1.3 ± 0.3 [symbol] |
| BIRC3 (o-IAP1) | 0.1 ± 0.3 | 3.6 ± 0.4 [symbol] | 1.6 ± 0.2 [symbol] | 2.6 ± 0.4 [symbol] |

Double strand Breast DNA Repair

| | | | | |
|---|---|---|---|---|
| BRCA1 | 1.0 ± 0.1 | 1.0 ± 0.1 | 6.6 ± 0.6 [symbol] | 2.1 ± 0.6 [symbol] |

Cytoplasmin DNA

[symbol]

| | | | | |
|---|---|---|---|---|
| AIM2 | 1.2 ± 0.2 | 1.3 ± 0.1 | 2.2 ± 0.2 [symbol] | 2.6 ± 0.4 |
| RIG1 | 1.6 ± 0.2 | 1.3 ± 0.2 | 2.0 ± 0.2 [symbol] | 1.4 ± 0.3 |
| STING | 1.3 ± 0.2 | 1.4 ± 0.2 | 1.0 ± 0.2 | 1.3 ± 0.3 |
| TLR9 | 1.6 ± 0.2 [symbol] | 1.3 ± 0.2 | 3.0 ± 0.3 [symbol] | 1.2 ± 0.2 |

Nrf2- [symbol] 1 Pathway

| | | | | |
|---|---|---|---|---|
| NRF2 (NFE2L2) | 1.4 ± 0.1 [symbol] | 1.1 ± 0.1 | 2.3 ± 0.1 [symbol] | 1.2 ± 0.2 |
| REAP1 | 0.9 ± 0.1 | 1.1 ± 0.1 | 1.6 ± 0.2 [symbol] | 1.0 ± 0.1 |

NFαS Pathway:

| | | | | |
|---|---|---|---|---|
| MAP4K4 | 1.1 ± 0.2 | 1.6 ± 0.2 | 2.0 ± 0.1 [symbol] | 1.1 ± 0.2 |
| MYD38 | 1.0 ± 0.2 | 2.0 ± 0.2 [symbol] | 3.5 ± 0.2 [symbol] | 1.4 ± 0.1 |
| NFKB1 | 1.6 ± 0.2 | 0.9 ± 0.2 | 1.8 ± 0.2 [symbol] | 1.6 ± 0.4 |
| TIRAP | 1.0 ± 0.2 | 2.2 ± 0.2 [symbol] | 2.3 ± 0.3 [symbol] | 1.3 ± 0.1 |

STAT Family:

| | | | | |
|---|---|---|---|---|
| STAT3 | 1.2 ± 0.2 | 1.9 ± 0.1 [symbol] | 3.0 ± 0.3 [symbol] | 1.0 ± 0.2 |
| STAT6 | 1.2 ± 0.2 | 1.3 ± 0.3 [symbol] | 1.6 ± 0.3 [symbol] | 1.1 ± 0.2 |

MAPK and [symbol] Pathway:

| | | | | |
|---|---|---|---|---|
| FOS | 1.3 ± 0.2 | 1.4 ± 0.3 | 1.4 ± 0.2 | 1.3 ± 0.3 |
| JUN | 1.6 ± 0.3 [symbol] | 1.6 ± 0.2 [symbol] | 2.3 ± 0.3 [symbol] | 1.3 ± 0.4 [symbol] |
| MAPKB (JNK1) | 0.6 ± 0.2 | 1.8 ± 0.2 [symbol] | 1.3 ± 0.1 | 1.3 ± 0.2 |

Cytokine

| | | | | |
|---|---|---|---|---|
| IL10 | 0.8 ± 0.2 | 3.3 ± 0.5 [symbol] | 1.6 ± 0.2 [symbol] | 4.2 ± 0.4 [symbol] |
| IL6 | 0.6 ± 0.3 | 1.8 ± 0.2 [symbol] | 2.6 ± 0.3 [symbol] | 1.9 ± 0.2 [symbol] |
| IL8 | 1.7 ± 0.2 [symbol] | 1.1 ± 0.2 | 2.2 ± 0.2 [symbol] | 1.4 ± 0.4 |
| TNFα | 1.8 ± 0.2 [symbol] | 2.2 ± 0.2 [symbol] | 3.6 ± 0.2 [symbol] | 2.3 ± 0.3 [symbol] |

Cell Ad [symbol] and Cell Migration Mo [symbol] :

| | | | | |
|---|---|---|---|---|
| ICAM1 | 0.9 ± 0.2 | 1.3 ± 0.2 | 2.6 ± 0.3 [symbol] | 1.6 ± 0.4 |
| PECAM1 | 1.1 ± 0.2 | 1.4 ± 0.2 | 1.2 ± 0.2 [symbol] | 1.2 ± 0.2 |
| SELE | 1.0 ± 0.1 | 1.1 ± 0.2 | 2.1 ± 0.3 [symbol] | 1.0 ± 0.2 |
| SELP | 1.7 ± 0.3 [symbol] | 1.3 ± 0.2 [symbol] | 1.3 ± 0.2 | 1.6 ± 0.3 [symbol] |
| VCAM1 | 1.5 ± 0.3 | 1.3 ± 0.2 [symbol] | 3.4 ± 0.3 [symbol] | 1.3 ± 0.2 |
| [symbol] | 1.3 ± 0.2 | 1.2 ± 0.2 | 1.6 ± 0.2 [symbol] | 1.1 ± 0.1 |

Growth Factors:

| | | | | |
|---|---|---|---|---|
| BMP2 | 1.9 ± 2.2 | 1.7 ± 0.2 [symbol] | 3.0 ± 0.3 [symbol] | 2.4 ± 0.2 [symbol] |
| BMP4 | 1.2 ± 0.2 | 1.9 ± 0.1 [symbol] | 2.6 ± 0.4 [symbol] | 1.4 ± 0.4 |
| VEGFA | 1.3 ± 0.2 | 1.9 ± 0.4 [symbol] | 0.1 ± 0.3 | 1.4 ± 0.3 |

Pluripotent [symbol] cell-related genes:

| | | | | |
|---|---|---|---|---|
| NANOG | 1.2 ± 0.3 | 1.4 ± 0.1 [symbol] | 1.2 ± 0.2 | 1.0 ± 0.2 |
| OCV4 | 1.2 ± 0.2 | 1.6 ± 0.2 [symbol] | 2.6 ± 0.2 [symbol] | 1.7 ± 0.1 [symbol] |
| GATA-4 | 1.1 ± 2.2 | 1.6 ± 0.2 [symbol] | 1.4 ± 0.3 | 1.3 ± 0.3 |

Relative levels of expression are averages for three [symbol] cal replicates and a standard deviation (°) p < 0.06 - against control cells, non-parametre U-test Mann- [symbol] [symbol]
indicates data missing or illegible when filed

Prolonged exposure of MCF-7 to oxidized DNA leads to a decrease in the intensity of the staining of individual cells with anti-TLR9 antibodies (FIG. 3D[2]). Earlier, similar type of the response gDNA and gDNA^{OX} was observed in cultured human fibroblasts [7]. All together, the data indicates that prolonged exposure to either gDNA or gDNA^{OX} leads to the decrease of the cellular levels of DNA sensors AIM2 and TLR9 and, possibly, to partial desensitization of these cells to effects of extracellular DNA.

### Example 10

### Exposure to gDNA^{OX} Induces Short-Term Oxidative Stress

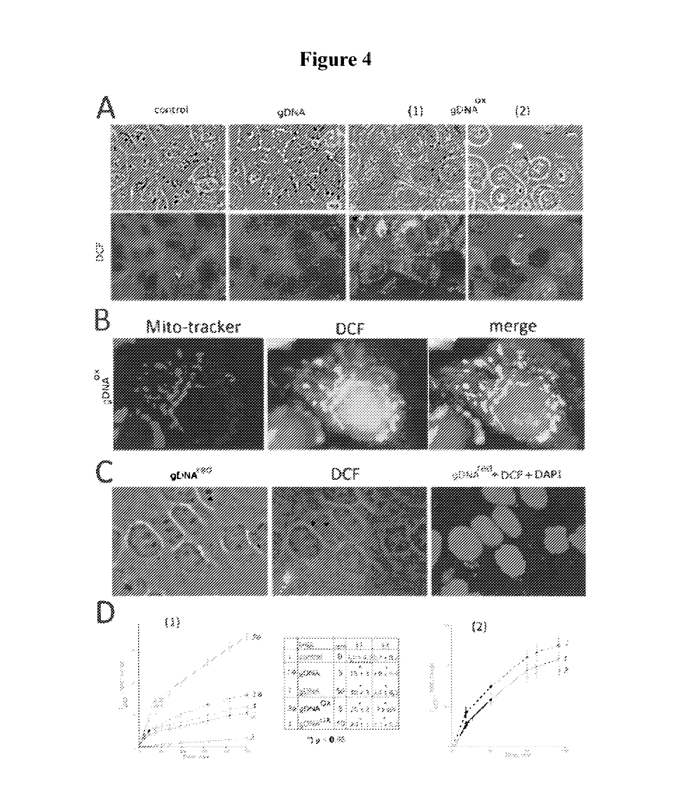

To study possible influence of gDNA and gDNA^{OX} on the intracellular levels of reactive oxygen species (ROS), the ROS were measured using dichlorodihydrofluorescindiacetate (H2DCFH-DA) dye that rapidly penetrates cell membranes, and gets trapped in the cytosol in its deacetylated form. Nonfluorescent DCFH transforms to fluorescent DCF by a variety of ROS radicals and, therefore, serves as a sensitive intracellular marker for oxidative stress [29]. FIG. 4A depicts the results of the ROS levels analysis in living cells. In untreated control cells, DCF dye diffusely associates with the surface of the cell, and may be removed from the membrane by PBS washing. Most common sources of ROS at cellular membrane are enzymes of NOX family [30]. In cells treated with gDNA (50 ng/mL), H2DCFH-DA stain visualizes both the membrane and some amount of intracellular granules. The PBS wash does not influence cytoplasmic granule staining. Patterns of DCF granules and labeled gDNA^{red} probe stains approximately overlap (FIG. 4C), possibly indicating that an interaction of gDNA with some cellular constituents stimulates ROS biosynthesis at the place of contact. This observation aligns well with previously stated hypothesis that ecDNA may somehow directly stimulate enzymatic activity of NOX proteins [5].

In cells treated with gDNA^{OX} (50 ng/ml), intracellular ROS-producing granules arise fast, and their numbers are substantially larger than in cells treated with gDNA (FIG. 4A, inset gDNA^{OX}[1]). These events are accompanied by changes in the morphology of MCF-7 cells, including an increase in size of nuclei and cytoplasmic swell. It is important to note that observed cellular responses are rapid and short-living. Described changes in staining patterns and cell morphology are seen only in case of sequential additions of H2DCFH-DA and gDNA^{OX} to MCF-7 media. When cells were pre-treated with gDNA^{OX} for 1 hour, then studied using a H2DCFH-DA dye, the number ROS-synthesizing granules seen in cells was lower and their intensities were less bright than in case of no pretreatment protocol (FIG. 4A inset gDNA^{OX} [2]). Even more interesting, in pre-treatment protocol, some cells stopped ROS biosynthesis at all, and became even less bright then untreated control cells (darker cells that are less fluorescent than the background (FIG. 4A inset gDNA^{OX} (b)).

The observed phenomena were independently confirmed in a study of DCF generation kinetics using quantification with a fluorescent reader (FIG. 4D). When MCF-7 cells were treated with DNA immediately after addition of H2DCFH-DA to the media, a dramatic increase in the intensity of DCF fluorescence was observed. These increases were at the highest rates of increase during first 20 minutes after the addition of DNA to the media (coefficient k1), then, with time, these rates drop (coefficient k2) (FIG. 4D[1], Table inset). k1 and k2 coefficients were dependent on type and concentrations of DNA treatment: gDNA^{OX} (5 ng/mL)>gDNA (5 ng/mL)>gDNA^{OX} (50 ng/mL)≧gDNA (50 ng/mL)>control. These effects were not seen when cells were pretreated with DNA for 1 hour before the addition of H2DCFH-DA (FIG. 4D[2]).

Taken together, the results of these experiments indicate that treatment with gDNA^{OX} rapidly induces ROS biosynthesis in MCF-7 cells. In parallel, the opposite process of the suppression of ROS generation, or ROS quenching, is initiated. As larger the amounts of gDNA^{OX} were added to the media, the more rapid was the development of ROS quenching.

A bulk of the intracellular ROS is generated by mitochondria. An increase in oxidative metabolism in mitochondria may lead to the diffusion of ROS into cytoplasm and subsequent increase in perimitochondrial detection of ROS by DCF. To test this hypothesis, cells exposed to 50 ng/mL of gDNA^{OX} for 30 minutes were sequentially stained with Mito-tracker (TMRM red) and DCF (FIG. 4B). A majority of Mito-tracker and DCF signal were located close to each other, with partially overlaps (yellow signal, FIG. 4B). In intact cells, H2DCFH-DA does not stain mitochondria (FIG. 4A, control). Based on observations of cells exposed to oxidized DNA, a majority of endogenous ROS is generated by mitochondria.

### Example 11

### Exposure to gDNA^{OX} Stimulates an Increase in the Levels of Oxidative Modification of Cell' Own DNA

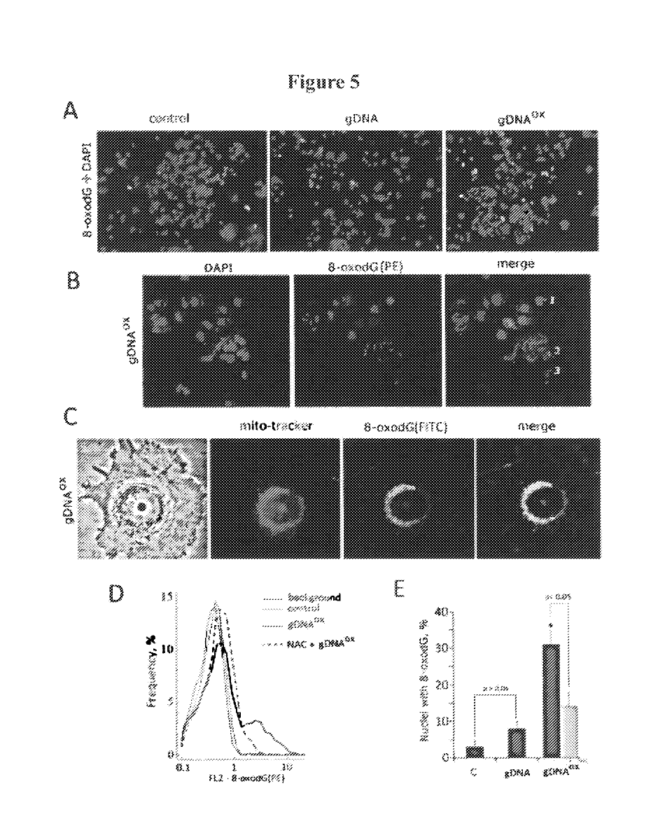

It is likely that intensive production of ROS observed immediately after exposure of cells to gDNA^{OX} may result in the damage to cellular DNA. To visualize this damage, fixed MCF-7 cells were stained with PE-labeled anti-8-oxodG antibodies (FIG. 5). As compared to non-treated control cells, in MCF-7 cultures treated with either gDNA or gDNA^{OX}, the amounts of stained cells were increased (FIG. 5A (×20). At larger magnifications, three types of staining patterns may be detected (FIG. 5B): (1)—nuclear staining; (2)—cytoplasmic staining; (3)—staining for micronuclei. In non-treated control populations of MCF-7 cells, PE-labeled anti-8-oxodG antibodies predominantly stain micronuclei. In populations treated with gDNA^{OX}, there was an increase in the amounts of cells with nuclear staining (FIG. 5E). As the previous experiments showed that gDNA^{red-OX} is located in cytoplasm and does not penetrate the nucleus, observed staining of nuclei shall be attributed to the damage of cell' own DNA.

An increase of mitochondrial biosynthesis of the ROS in gDNA^{OX} exposed cells demonstrated above (FIG. 4B) may lead to an increase in the level of oxidation in mitochondrial DNA that, in turn, may explain observed cytoplasmic staining for gDNA^{red-OX} shown at FIG. 2C. On FIG. 5C, one may see that some 8-oxodG signals do not merge with gDNA^{red-OX}. In cells pretreated with antioxidant N-acetyl-cysteine (NAC) (0.15 mM) for 30 minutes before exposure to gDNA^{OX}, the levels of oxidation in cellular DNA were substantially lower than in cells not treated with NAC (FIGS. 5D and 5E).

### Example 12

### Exposure to gDNA^{OX} Stimulates an Increase in Strand Breaks in Cell' Own DNA

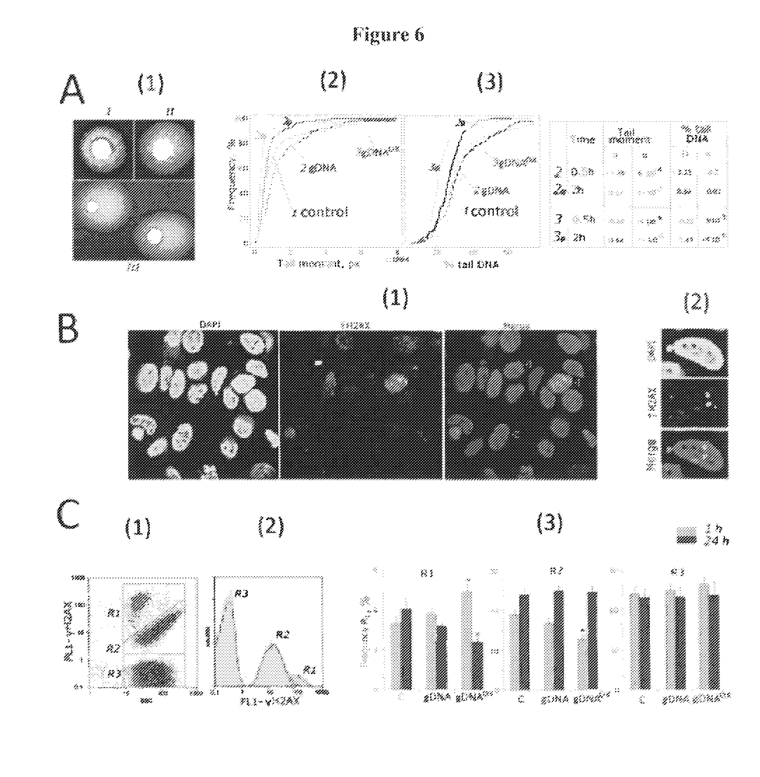

One of well-known feature of DNA oxidation is an accumulation of single- and double strand DNA breaks (SSBs and DSBs). To quantify SSBs and DSBs in MCF-7 cells exposed to either gDNA or gDNA^{OX}, comet electrophoresis was employed in alkaline conditions (FIG. 6A). Three types of nuclei were enumerated: nuclei with intact DNA (FIG. 6A [1], Type I); nuclei with some degree of chromatin fragmentation (Type II); nuclei with substantial fragmentation of DNA (Type III). In majority of cases, the nuclei of non-treated control are classified as either Type I or Type II, while Type III nuclei are seen predominantly in cells treated with gDNA^{OX}. Depending on how long the cells were exposed to gDNA^{OX}, the proportions of Type III nuclei may differ. FIG. 6A also presents the comet tail moments [2] and % tail DNA [3]. After 30 minutes of incubation of MCF-7 cells with gDNA^{OX}, the amounts of DNA breaks drastically increase, while similar treatment with gDNA leads to moderate elevation of chromatin fragmentation levels. After 2 hours of incubation either with gDNA or gDNA^{OX}, the amounts of DNA breaks decrease, and their number falls to below of that found in respective gate-specific populations in non-treated control cells.

Observations described above were independently confirmed using another common technique for visualization of DSBs, an immunostaining with antibodies against the histone γH2AX, phosphorylated by serine-139. This form of H2AX is known to rapidly accumulate at DNA loci flanking the DSB site [31]. MCF-7 cells stained with FITC-conjugated antibodies to Ser-139 phosphorylated histone γH2AX are shown at FIG. 6B [1]. Stained slides also included three different cell populations of γH2AX positive cells. In this experiment, cells were classified as Type I cells when they had multiple phospho-γH2AX foci. Most of the γH2AX positive cells were classified as Type 2 cells (between 2 and 10 distinct γH2AX foci per cell), and Type 3 cells with no signs of the focal phospho-γH2AX staining.

In anti-γH2AX staining, overall fluorescence intensity of the cell is proportional to the number of γH2AX foci per cell, and, therefore, to amount of DSBs. Using FACS, three gated areas, R1 to R3, were studied (FIG. 6C[1,2]). Cells within gate R1 have largest FL1 (γH2AX); this is interpreted as multiple DSBs (Type 1 cells, FIG. 5B). Gate R2 contains cells with not numerous γH2AX (Type 2 cells). Gate R3 contains the largest number of cells; most of these cells are intact with no DSBs (Type 3 cells). In MCF-7 cultures, an exposure to gDNA^{OX} (1 h) leads to a 1.5-folds increase in the number of cells within gate R1 that is paralleled by a decrease in the number of cells within R2. After 24 hours of exposure to gDNA^{OX}, the amounts of cells with multiple DSBs decrease to the levels below that that in non-treated control cells (FIG. 6C[3]). A treatment with gDNA evokes similar, but less pronounced type of cellular response that in its magnitude does not reach significance when compared to non-treated control cells (p>0.05).

These observations indicate that, in MCF-7 cells, short-term exposure to gDNA^{OX} results in both single- and double strand DNA breaks. Longer durations of the treatment (between 2 and 24 hours) evoke some type of compensatory response that leads to a decrease in the levels of chromatin fragmentations across cell populations.

The drop in the proportion of DSB-containing cells after short-term exposure to oxidized or control DNA may be explained either by the repair of the breaks, or by apoptosis/detachment of damaged cells, or both. To evaluate these possibilities, cells that remain in the media after its removal from cell layer, and cells removed from the layer after PBS wash were enumerated. In cultures exposed to oxidized DNA for 2 hours, the proportion of detached cells remained similar to that in cultures exposed to genomic DNA and non-treated control cultures (approximately 2% of total amount of cells in given culture). Similar results were obtained in experiments aimed at direct evaluation of apoptosis (see below). Therefore, it is likely that the decrease in the proportion of cells with DSBs observed after exposure to gDNA or gDNA^{OX} is due to an increase in DNA repair.

### Example 13

### Exposure to gDNA^{OX} Leads to an Increase in Genome Instability

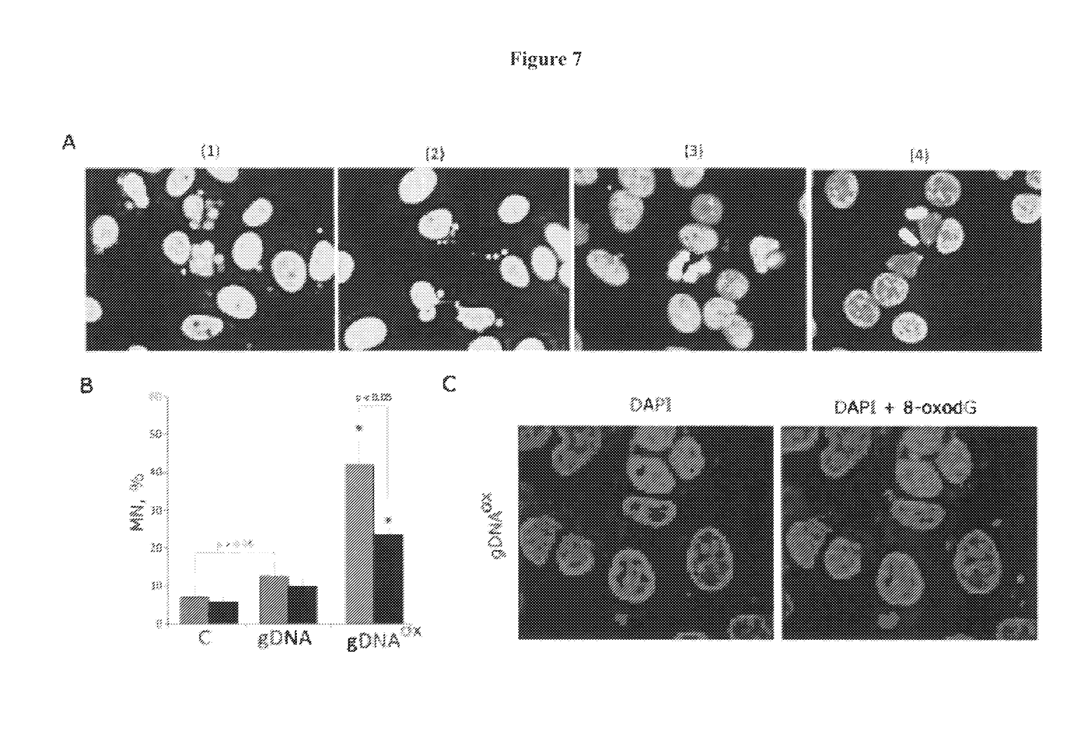

Single- and double strand DNA breaks are known to result in the loss of chromosome stability that is especially prominent in actively proliferating cells [32]. A thorough study of the nuclei of the cells incubated with gDNA^{OX} revealed pronounced chromosome instability (FIG. 7). At concentrations of 50 ng/mL, an exposure of actively proliferating, low confluency MCF-7 cells to gDNA^{OX} results in the formation of multiple micronuclei (FIG. 6A[1]) and other nuclear anomalies such as nucleoplasmic bridges and nuclear buds (FIG. 7A[2]), as well as in decondensation of mitotic chromosomes (FIG. 7A[3]). All of these events are signs of profound replication stress that is known to develop in actively proliferation cell cultures undergoing various stress treatments [32]. Similarly treated cell cultures with lower proportions of proliferating cells, for example, confluent or serum starved cultures show substantially lesser amounts of chromatin changes. Proportions of micronuclei-containing cells in cultures grown in varying conditions are show at FIG. 7B. In non-treated control MCF-7 cells, the frequency of cells with micronuclei was around 7%, a number that is similar to that reported in other studies [33]. In actively proliferating cultures exposed to gDNA^{OX}, the micronuclei were detected in about 40% of cells. Exposure to gDNA also leads to increase in the amounts of cells with micronuclei, but in this case an increase is not significant. Many micronuclei formed after the treatment with gDNA^{OX} were positively stained for both PE-labeled anti-8-oxodG (FIG. 5B and FIG. 7C) and anti-phospho-γH2AX antibodies that highlight DSBs (FIG. 6B [2]).

These observations indicate that, in MCF-7 cells, an exposure to gDNA^{OX} induces genome instability that is, most likely, secondary to accumulation of large the amounts of SSBs and DSBs.

### Example 14

### Exposure to gDNA^{OX} Arrests Cell Cycle

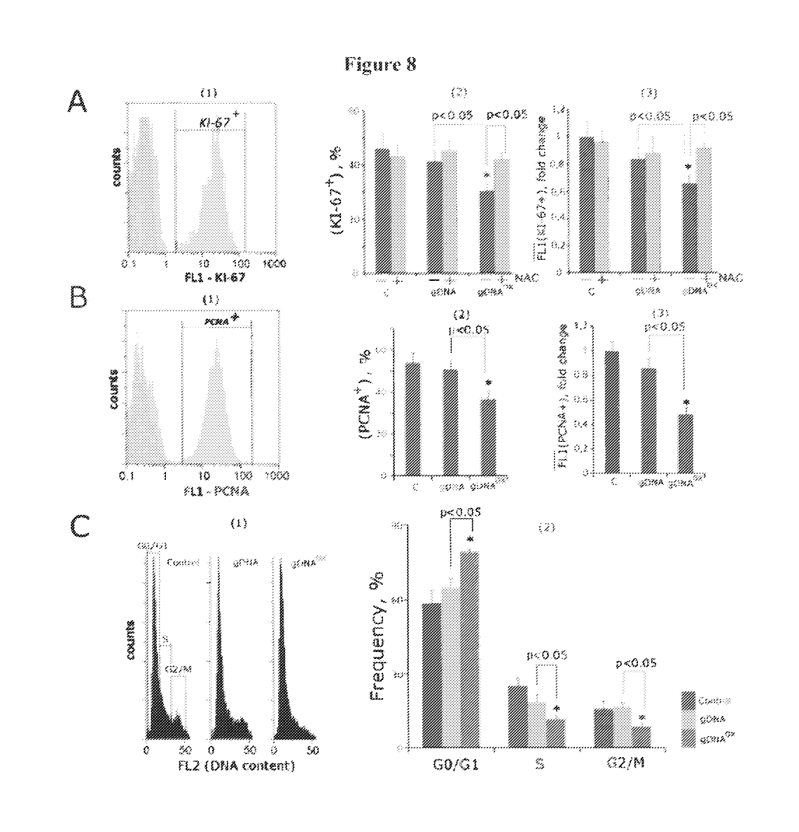

One of the most important consequences of genome instability is the block of cell proliferation due to activation of the DNA damage checkpoints. Cell cycle-related consequences of exposure to or gDNA were studied in MCF-7 cells that were harvested 48 hours after addition of DNA (50 ng/mL) to the media (FIG. 8).

To investigate these cultures, cells were stained with antibodies to the proliferation markers Ki-67 and PCNA [34,35] and enumerated by FACS. Additionally, cell counts were also performed after DNA-specific propidium iodide (PI) treatment. FIG. 8A shows the distribution of the cells with various Ki-67 contents. In control MCF-7 cultures, Ki-67 stains approximately 45% of cells. After exposure to gDNA^{OX}, the proportion of Ki-67-positive cells decreased to 30% (FIG. 8A[2]). These decreases were paralleled by the decrease in mean fluorescence intensity per each Ki-67-positive cell by 40% that is indicative of the decrease in amounts of Ki-67 in individual cells. Similar results were obtained using another well-known marker of proliferation, PCNA (FIG. 8B[1-3]). It seems that observed block of proliferation is ROS-dependent, as the changes in KI-67 staining of the cells pre-treated with antioxidant NAC (0.15 mM) and exposed to same amounts of oxidized DNA were not significant (FIG. 8C[2,3]).

The data collected after the staining with propidium iodide (PI) point to similar direction (FIG. 8C[1]). After exposure to gDNA^{OX}, the proportion of G0/G1 cells increased, while proportions of the cells in S- and G2/M phases decreased (FIG. 8C[2]). These observations indicate that, in a substantial proportion of previously proliferating MCF-7 cells, the exposure to gDNA^{OX} and, to a lesser degree, to gDNA blocks the cell cycle in G0/G1.

This line of evidence was also supported by qRT-PCR analysis at the level of mRNA encoding inducible cell cycle arrest proteins, including CDKN2A (p16INK4), CDKN1A (p21CIP1/WAF1) and TP53 (Table 1). Cell cycle changes evoked by treatment with gDNA were similar to those of gDNA^{OX}, but substantially less pronounced.

### Example 15

### Exposure to Either gDNA^{OX} or gDNA Supports Cell Survival

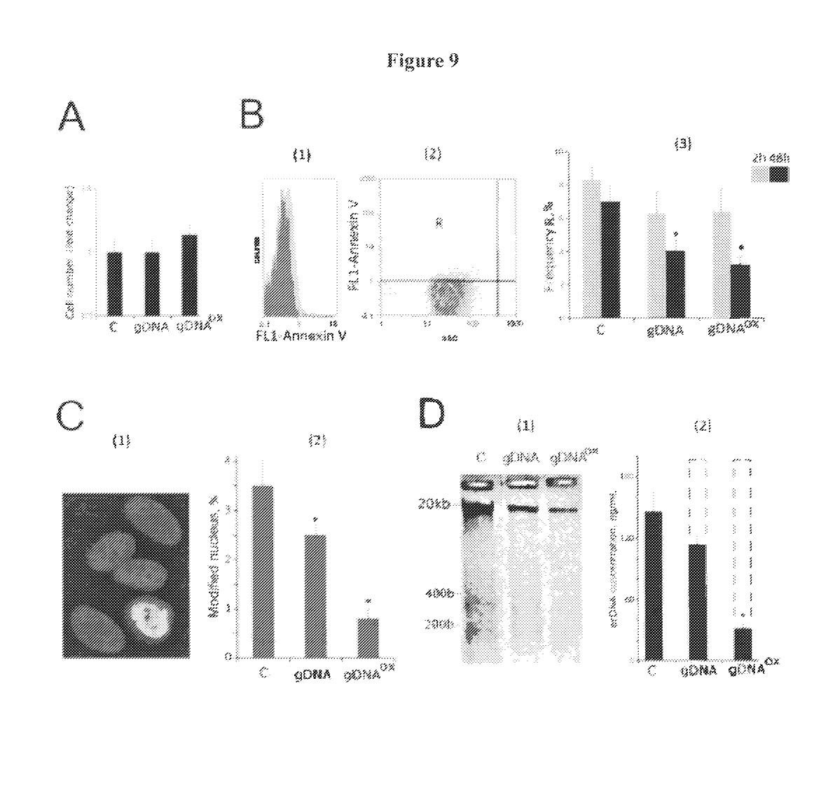

It was noted that the total amount of cells harvested 48 hours after exposure to gDNA^{OX} or gDNA were similar to those of non-treated control populations (FIG. 9A). As the proliferation activities of cells treated with either gDNA^{OX} or gDNA were, at least in part, blocked (FIG. 8), it was important to evaluate overall levels of cell death in all studied populations.

To quantify cells in early apoptosis, FITC-conjugated Annexin V was used (FIG. 9B[1-3]). After two hours of exposure either gDNA^{OX} or gDNA, the proportion of the apoptotic cells went down approximately by 25%, but observed changes had not reached significance (p>0.05)). However, after 48 hours of exposure to either gDNA^{OX} or gDNA, the proportion of apoptotic cells in treated cultures decreased to the levels twice less than in control MCF-7 cultures.

To evaluate overall levels of cell death in all studied populations, nuclear morphology was evaluated in all populations after staining with Hoechst33342 (FIG. 9C [1,2]). If condensed and fragmented chromatin was detected, the cell was marked as apoptotic. After exposure to gDNA^{OX} (48 hours, 50 ng/mL), the amount of cells with apoptotic nuclei decreased three folds.

To further assess various aspects of cell death, ecDNA was extracted from cell-free media conditioned by non-treated control cells and cells treated either with gDNA or gDNA^{OX} for 48 hours (50 ng/mL). Extracted DNA fragments were analyzed by gel electrophoresis to assess their size distribution (FIG. 9D[1]). The length of DNA fragments extracted from cell-free media conditioned by non-treated control cells, varied between 15 kb and 0.1 kb, and included visible mono- and dinucleosome bands that are contributed to the ecDNA pool by dying apoptotic cells [36]. In cells treated either with gDNA or gDNA^{OX}, these bands were less prominent. The decrease in relative abundance of mono- and dinucleosome bands was in concert with the overall decrease in total amounts of ecDNA extracted from cell-free media and quantified using RiboGreen stain (FIG. 9D[2]). In media of MCF-7 cells exposed to exogenous DNA, the final concentrations of ecDNA should be around 190 ng/mL (a sum of concentrations of endogenously produced DNA at 140 ng/mL and added DNA at 50 ng/mL); However, cell-free media of cells treated with exogenous DNA had substantially lower concentrations of DNA, in fact, after treatment with gDNA^{OX}, these concentrations were 1.7 times lower than expected. After treatment with gDNA^{OX}, these concentrations were 6 times lower than expected. These drastic drops in DNA concentrations may be explained by the decrease of overall levels of apoptosis and DNA release in gDNA or gDNA^{OX} treated cultures.

FIG. 9 presents evidence that in gDNA^{OX} treated MCF-7 cultures and, to lesser degree, in gDNA treated cells, the levels of cell death substantially decrease as compared to untreated controls. Additional supportive evidence for this statement is presented in Table 1 that summarizes the changes in expression levels for mRNAs encoding cell survival and DNA repair related proteins. In two hours after adding gDNA^{OX} to MCF-7 culture, levels of mRNA for BCL2, BCL2A1 (Bfl-1/A1), BCL2L1 (BCL-X), BIRC3 (c-IAP1) and BRCA1 increase 1.2 to 6.4 folds, and stay elevated for at least 48 hours. In case of treatment with gDNA, these genes also tend to increase their mRNA biosynthesis, up to 1.9-3.5 times, but these changes in expression levels are delayed as compared to the treatment with gDNA^{OX} and reach significance only after 48 hours. Interestingly, in case of treatment with gDNA, the expression levels of mRNA encoding for key component of DSB repair machinery BRCA1, were not altered.

### Example 16

### Exposure to Either gDNA^{OX} or gDNA Leads to a Decrease in Activity of NRF2 and an Increase in Activity of NF-kB and STAT3

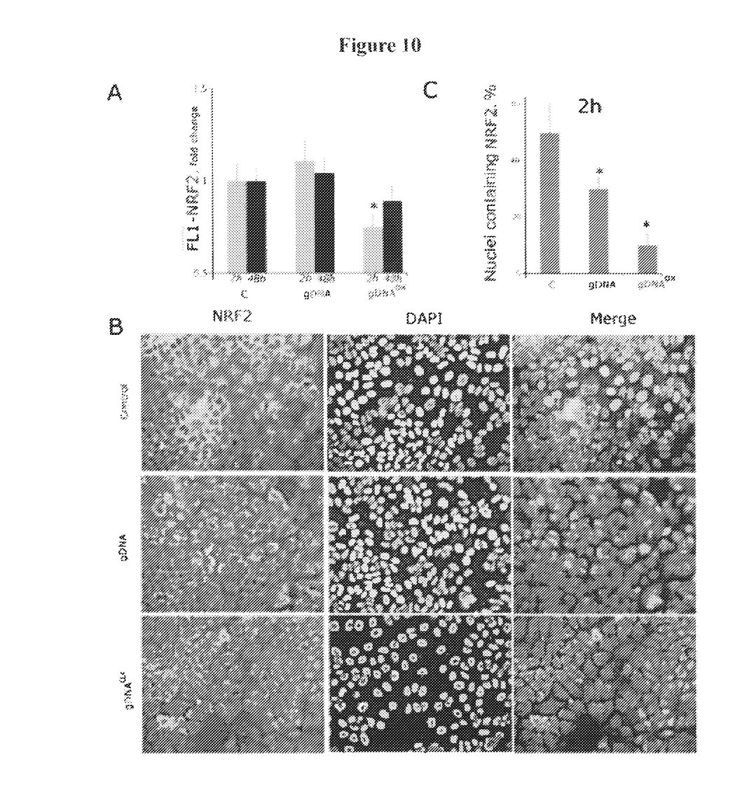

NF-E2-related factor 2 (NRF2) is known to participate in the development of adaptive response in fibroblasts and mesenchymal stem cells cultivated in the presence of gDNA^{OX} [5,7]. After 2 hours of exposure of MCF-7 cells to gDNA^{OX}, the levels of NRF2 mRNA increase (Table 1). At the same time point, there is an increase in the expression of the gene KEAP1 that encodes for a cytoplasmic protein partner of NRF2, capable of blocking its transcription factor activity [37]. As evident from FACS data, protein levels of NRF2 after treatment with gDNA do not change (FIG. 10A). An exposure to gDNA^{OX} for 2 hours leads to a decrease of NRF2 levels. Fluorescent microscopy studies showed that exposure to gDNA^{OX} leads to a change in the NRF2 staining pattern. In non-treated control MCF-7 cells, NRF2 is located both in the nucleus (˜50% of cells) and in the cytoplasm (most of the cells), while in cells exposed to gDNA^{OX} NFR2 is found exclusively in the cytoplasm (FIG. 10B), thus, indicating suggesting that its transcriptional activator function is blocked.

NF-κB and STAT3 control the expression of anti-apoptotic and cell cycle control and proliferation genes. Both of these transcriptional factors are activated in response to various kinds of stress. In particular, NF-κB and STAT3 were found to play pivotal roles in various aspects of tumorigenesis [38,39]. Here, an analysis is presented of activity of these two transcription factors in cells exposed to either gDNA or gDNA^{OX} NF-κB.

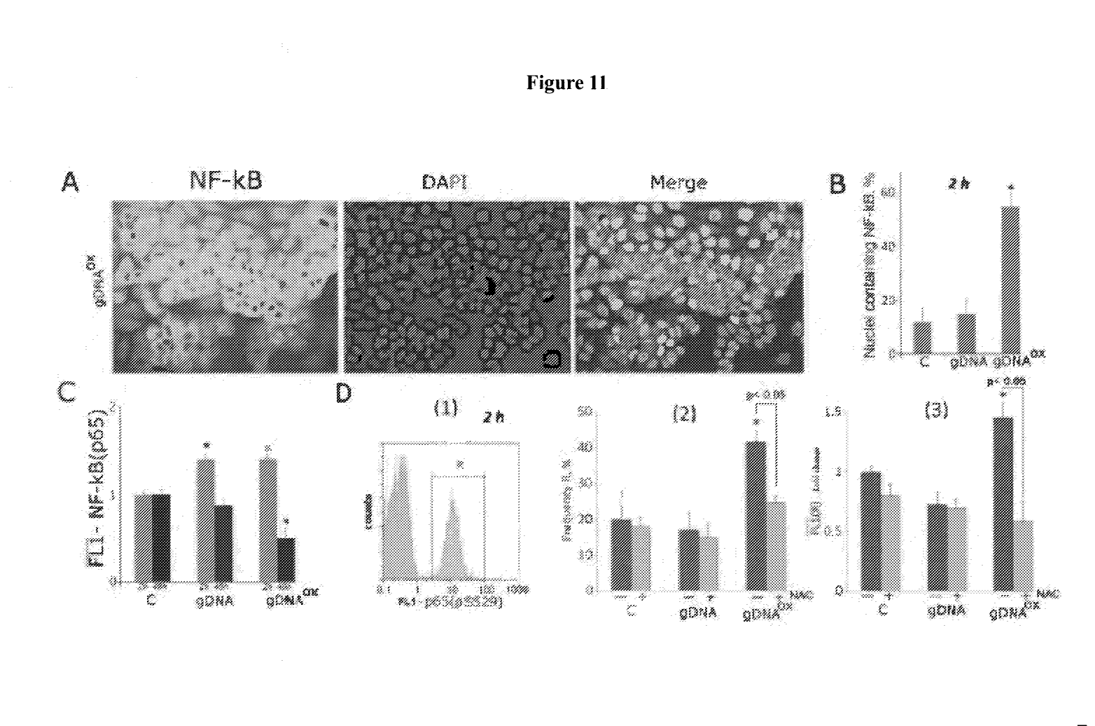

The exposure to gDNA^{OX} leads to a rapid, 1.8-3.6 fold increase in the levels of mRNAs encoding components of the NF-κB pathway, including MAP4K4, MYD88, NFKB1 and TIRAP (Table 1). The effects of exposure to gDNA are seen substantially later, at 48 hours post exposure (MAP4K4, MYD88 and TIRAP). After 2 hours of exposure to either gDNA or gDNA^{OX}, the amount of NF-κB (p65) proteins increase 1.5 fold (FACS, FIG. 11C), and decrease 48 hours later. Fluorescent microscopy evaluation of gDNA^{OX}-treated MCF-7 cells confirms activation of NF-κB as evident from the translocation of this factor into the nucleus (FIG. 11A). After 2 hours of exposure, the fraction of MCF-7 cells with nuclear staining for NF-κB increases from 12% to 56% (FIG. 11B).

It is known that NF-κB (p65) is activated by phosphorylation, which plays a key role in the regulation of its transcriptional activity and is associated with nuclear translocation. For instance, upon treatment with TNFa, Ser529 of p65 is phosphorylated by casein kinase II [40]. Flow cytometry quantification (FIG. 11D) demonstrates that exposure to gDNA^{OX} leads to an increase of the proportion of cells that contain Ser529-phosphorylated p65, thus, confirming that NF-κB in these cells is transcriptionally active [40]. The exposure to gDNA does not increase the proportion of cells with Ser529-phosphorylated p65. The pre-treatment with antioxidant NAC at 0.15 mM for 30 minutes before addition of same amount of oxidized DNA prevented an increase in the levels of Ser529-phosphorylated p65 that remained similar to that in control cells (FIG. 11D [2,3]). Therefore, it may be concluded that oxidized DNA dependent activation of NF-κB is mediated by an increase in local production of ROS.

### STAT3.

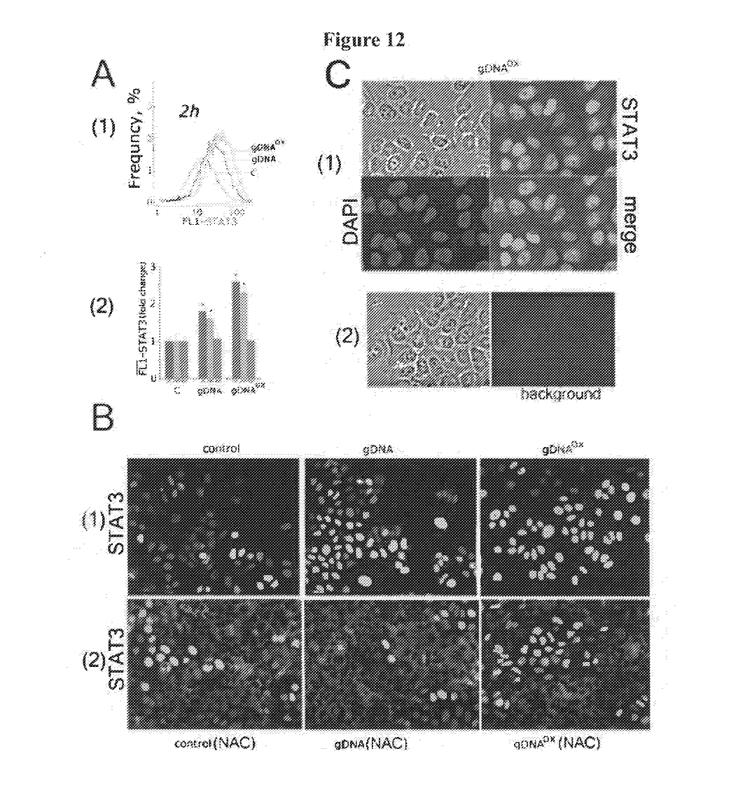

Two hours exposure to gDNA^{OX} also leads to an increase in the expression of mRNA for STAT3 and STAT6 (3 and 1.6 fold, respectively) (Table 1), while exposure to gDNA results in significant activation of STAT3 and STAT6 only at the 48 hour time point. Both FACS and fluorescent microscopy show that non-treated control MCF-7 cells express substantial amounts of STAT3 (FIG. 12A[1,2], 12B[1], 12C). Importantly, in these cells STAT3 is located exclusively in the nuclei. These observations indicate that STAT3 in active in control MCF-7 cultures. Published studies describing activity of Stat3 in MCF-7 contradict each other. Some authors showed that in MCF-7 Stat3 is phosphorylated and located in the nuclei [41]. Other studies failed to detect activity of Stat3 in MCF-7 [42]. Stat3 activity may change in response to growth factors and cytokines [38,39]. Therefore, observed disagreements may be explained by differing cultivation conditions, in particular, by type of the serum supplementation. Interestingly, supplementation of the media with antioxidant NAC leads to decrease in activity of Stat3 (FIG. 11B[2]).

After 2 hours of exposure to either gDNA^{OX} or gDNA, the amounts of STAT3 increase, with no changes in its localization. In 24 hours, the amounts of STAT3 protein start to decrease and in 48 hours after the addition of DNA, samples reach their initial levels (FIG. 11A[2]). In the case of exposure to gDNA^{OX}, these effects are more pronounced than in the case of gDNA. The pre-treatment with antioxidant NAC at 0.15 mM for 30 minutes before addition of same amount of oxidized DNA prevented activation of STAT3.

Both gDNA^{OX}- and gDNA-induced activation of NF-κB and STAT3 leads to an increase in the expression levels of genes encoding components of MAPK and JNK/p38 pathway: FOS, JUN and MAPK8 (JNK1). In parallel, an increase in the expression of genes that encode soluble cytokines (Table 1). For IL10, IL6, IL8 and TNFa was observed, the levels of mRNA increase 1.8-5.3 folds; two hours after adding DNA sample to the media, in gDNA^{OX}-treated MCF-7 cells, the levels of these mRNAs are 2-3 times higher than those in cells treated with gDNA. Additionally, the expression stimulating effects of gDNA^{OX} on cell adhesion and migration molecules ICAM1, PECAM1, SELE, SELP, VCAM1, and RHOA, growth factor encoding genes VEGFA, BMP4 and BMP2 and pluripotent stem cell-related genes NANOG, OCT4 and GATA-4 (Table 1) were observed.

High levels of cell-free DNA were found in cancer patients and in relevant in vivo models previously [43]. Moreover, substantially larger degrees of cfDNA fragmentation were observed both in cancer patients and in nude mice xenograft models, pointing to apoptotic cells as a possible source of cfDNA [44]. It is likely that the DNA released from dying cells as a result of oxidative insult. i.e. irradiation or chemotherapy-associated oxidative stress, is also damaged. Thus, all over the body, cells experience both an increase in the quantities of extracellular DNA and have increased proportion of damaged/unusual nucleotide bases within extracellular DNA fragments.

The aim of this study was to model an event that is naturally occurring in the body of patients exposed to cell death-inducing antitumoral therapy, an increase in the level of damaged, circulating DNA released from dying cells. As the model cell line, the estrogen-sensitive breast adenocarcinoma cell line MCF-7 was selected because it is particularly well characterized and widely accepted for cancer studies. Media conditioned by MCF-7 cells contains substantially larger amounts of extracellular DNA (140 ng/mL) as compared to a variety of normal cells that were profiled previously, including fibroblasts [7], endotheliocytes [15] and mesenchymal stem cells [5,6] (6-30 ng/mL).

One of the most important conclusions of this study is that normal, non-oxidized extracellular DNA penetrates the cells, but remains at the cytoplasmic foci close to the membrane. The number of these foci depends on the properties of extracellular DNA, in particular, on the degree of its enrichment in guanine and cytosine. It is likely that the binding of extracellular DNA to the cell membrane is mediated by receptors with varying affinities to different DNA sequences. It is also possible that the kinetics of ecDNA binding to the surface of MCF-7 cells differ from that of normal cells, due to larger concentrations of ecDNA in the media.

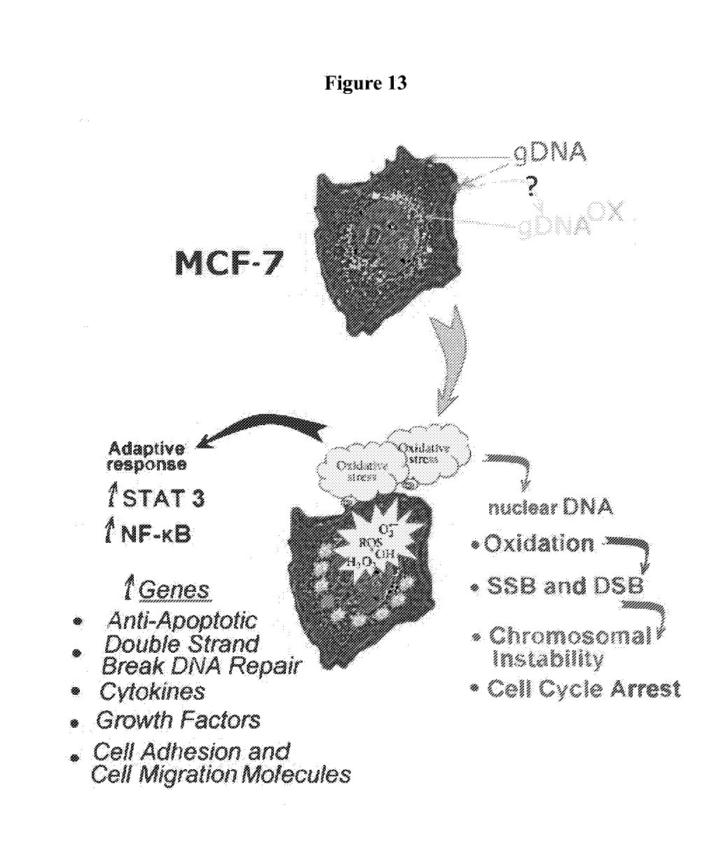

Intracellular distributions of oxidized and regular genomic DNA differ (FIG. 12). The fragments of gDNA^{OX} are located closer to the nucleus than similarly prepared fragments of regular gDNA (FIG. 2 A-C). An increase in expression of early endosomal marker EEA1 indicates that most likely mechanism for gDNA^{OX} penetration into the cells is through endocytosis (FIG. 2D). Some fraction of non-oxidized genomic DNA is also found at perinuclear locations (FIG. 2B); this is possibly due to secondary oxidation of DNA at the points of focal contact with the cell surface [5]. This hypothesis is supported by the local activation of ROS biosynthesis at DNA-associated foci (FIG. 4C). After oxidation, genomic DNA may be delivered inside the cell through the same pathway as gDNA^{OX} (FIG. 13).

After delivery into the cytoplasm, gDNA^{OX} immediately induces the burst of ROS (FIG. 4A). So far, not much is known about the particular mechanism that connects gDNA^{OX} to ROS-generating cascades. However, this data indicates that gDNA^{OX} induces the production of ROS by mitochondria (FIG. 4B).

The perinuclear production of ROS leads to either the direct damage to the genomic DNA of affected cells or to the increase in nuclear pool of free 8-oxodG that may affect genomic DNA of the cell through its salvage and incorporation into DNA [45,46]. In any case, exposure to gDNA^{OX} leads to an increase of 8-oxodG content in mitochondrial DNA (FIG. 5C), in the nuclear staining for 8-oxodG (FIG. 5) and the amounts of SSBs and DSBs in cell' DNA (FIG. 6). In turn, the accumulation of DNA breaks blocks cell proliferation through activation of checkpoints (FIG. 8). In addition, an increase in other signs of genome instability was observed, in particular, the number of micronuclei and other nuclear anomalies such as nucleoplasmic bridges and nuclear buds (FIG. 7). Therefore, the overall trend of MCF-7 cells response to exposure to gDNA^{OX} is an increase in the levels of damage to the cell' own DNA followed by the block of the division, and possibly, activation of DNA repair machinery.

Importantly, a burst in ROS biosynthesis that is observed in the first 30 minutes after adding gDNA^{OX} to the media is accompanied by an increase in anti-oxidant responses. After an hour of MCF-7 incubation with gDNA^{OX}, the levels of ROS biosynthesis drop below those seen in control, non-exposed cells (FIG. 4). Interestingly, the antioxidant responses of MCF-7 cells do not depend on activity of NRF2, a basic leucine zipper redox-sensitive transcriptional factor that plays a center role in ARE (antioxidant response element)-mediated induction of phase II detoxifying and antioxidant enzymes. In noncancerous cells treated with gDNA^{OX}, NRF2 mediates a set of adaptive responses [5,7]. Moreover, in MCF-7, NRF2 remains inactive despite nuclear translocation of oxidant-sensitive transcription factor NF-kB that controls expression of genes involved in immune and inflammatory responses. Crosstalk between NRF2 and NF-κB is an area of extensive interest. Typically, activation of NRF2 is accompanied by the block of NF-κB signaling pathways, and vice versa [47,48]. Exposure to gDNA^{OX} leads to activation of NF-κB, evident from an increase in mRNA levels for the components of NF-κB signaling pathway, elevation in the levels of p65 and its active, phosphorylated isoform as well as the nuclear translocation of p65, observed in 60% of cells (FIG. 11). In addition to the activation of NF-κB, exposure to gDNA^{OX} results in the upregulation of STAT3, known to promote the development and progression of some types of cancers [38,39]. After exposure of MCF-7 cells to gDNA^{OX}, the levels of both STAT3 mRNA and its protein increase approximately 2.5 folds [FIG. 12]. Interestingly, the transcription factor STAT3 has recently been found to suppress mtROS production independent of its nuclear factor activity [49].

Concerted activation of NF-kB and STAT3 is followed by an increase in expression levels of genes associated with cell survival. After 48 hours of exposure to gDNA^{OX}, a decrease in MCF-7 cell death was observed. These effects were seen notwithstanding an initial burst in ROS biosynthesis and extensive DNA damage observed in the beginning of the treatment with oxidized DNA. In gDNA^{OX}-treated cultures, a decrease in cell proliferation is paralleled by a decrease in cell death events, reflected by the lack of net change in the total amounts of cells in the culture wells (FIG. 9).

It seems that the effects of oxidized DNA are, at least in part, mediated by transient increase in the perimitochondrial levels of ROS. This is evident from experiments with experiments on cells pretreated with antioxidant NAC that precludes or substantially decreases the magnitude of gDNA^{OX}-dependent effects, in particular, the genomic DNA oxidation (FIG. 5 D,E), the block of the cell cycle (FIG. 8) as well as the activation of NF-kB (FIG. 11) and STAT3 (FIG. 12).

Taken together, this study indicates that exposure to oxidized DNA increases survivability of the tumor cells. These effects have substantial therapeutic relevance, as typical antitumoral therapy leads to massive cell death that, in many instances, includes a substantial oxidative damage related component [50], and, therefore, contributes to the release of oxidized DNA. Additionally, even in untreated tumors, the high endogenous levels of reactive oxygen species [51,52] results in increased levels of apoptosis that, in turn, increases the amounts of oxidized DNA that, in turn, leads to a homeostatic return to balance through stimulated increase in cell survival. This logic is consistent with the findings of Iwasa Y et al., that high rates of apoptosis within the tumor eventually leads to a higher incidence of pre-treatment resistance rather than what would be expected based on the size of the tumor only [53]. Moreover, this study suggests that oxidative stress-associated cell death, observed in many other chronic conditions [54] may be directly linked to tumorigenesis through associated increase in cell survival.

In conclusion, oxidized extracellular DNA released by dying tumor cells may stimulate survival of tumor cells. Importantly, in cells exposed to oxidized DNA, a suppression of cell death is accompanied by an increase in the markers of genome instability. Survival of cells with an unstable genome may substantially augment progression of malignancy. The model that describes the role of oxidized DNA released from apoptotic cells in tumor biology is depicted in FIG. 13.

### Example 17

### gDNA^{OX} in Patients with Chronic Diseases

To correctly design an experiment, it was imperative to use gDNA^{OX} with 8-oxo-dG content corresponding to that of 8-oxo-DG content in cell-free DNA of patients with chronic diseases. To this end, LC/MS quantification of 8-oxo-dG was performed in cfDNA fractions extracted from the plasma of two breast carcinoma patients and one patient with acute myocardial infarction. The range of 8-oxo-dG enrichment within cfDNA of breast carcinoma patients was 160-165 8-oxo-dG bases per 10⁶ nucleotides, while in the patient with the heart attack 410 8-oxo-dG bases per 10⁶ nucleotides was observed. The levels of 8-oxo-dG in intact gDNA were below the sensitivity of assay that was at 0.1 base of 8-oxo-dG per 10⁶ bases. After treatment with 300 mM H₂O₂/Fe²⁺/EDTA, the concentration of 8-oxo-dG gDNA^{OX} was 400 bases per million and, therefore, approximately within the range of 8-oxo-dG content in cfDNA of the patient with chronic diseases.

### Example 18

### DNA Oxidation In Vitro

Genomic DNA was extracted from HEFs as described above and evaluated by agarose gel electrophoresis for purity and fragment size. Controlled hydrolysis of the DNA by DNAse I (Invitrogen, USA) was performed until the length of the DNA fragments was reduced below 15 kb. The resulting DNA preparation (100 μg/mL) was exposed to a solution of 300 mM H₂O₂ with 10 μM Fe²⁺ and 10 μM EDTA in the dark for 30 minutes at 25° C. (gDNA^{OX}). Modified DNA was precipitated with 2 volumes of ethanol in the presence of 0.3 M CH₃COONa. The precipitate was washed twice with 70% ethanol, then dried and dissolved in water. The resulting DNA concentrations were measured by U V analysis.

### Quantitation of 8-Hydroxy-Deoxyguanosine (8-Oxo-dG) Levels

The samples of DNAs were dissolved in 20 μL water of HPLC quality and enzymatically digested in the following manner. After addition of 2.3 μL of 100 mM MgCl₂ and 0.5 μL if 1 M Tris•HCl (pH 7.4) to DNA solutions, 0.5 μL 2000 U/μL DNAse I was added for 1 hour incubation at 37° C. After adjusting the pH to 5.2 with 0.5 μL if 3 M sodium acetate (pH 5.2), the fragmented DNA was digested with 1 μL of NP1 (1 unit/μL) for 1 hour. After bringing the acidic pH back to neutral with 2.3 μL if 1 M Tris•HCl (pH 8.0), 0.5 μL of AP (1 unit/μL) was added, followed by 1 hour incubation. Quantitative analysis of oxidized deoxyguanosine in the mixture was determined by ESI-MS/MS using AB SCIEX 3200 Qtrap machine.

It is understood that the examples and embodiments described herein are for illustrative purposes only and that various modifications or changes in light thereof will be suggested to persons skilled in the art and are to be included within the spirit and purview of this application.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence was specifically and individually indicated to be so incorporated by reference.

### REFERENCES

1. Ermakov A V, Konkova M S, Kostyuk S V, Izevskaya V L, Baranova A, Veiko N N (2013) Oxidized extracellular DNA as a stress signal in human cells. Oxid Med Cell Longev 2013: 649747.
2. Efremova L V, Alekseeva A Y, Konkova M S, Kostyuk S V, Ershova E S et al. (2010) Extracellular DNA affects NO content in human endothelial cells. Bull Exp Biol Med 149: 196-200. doi:10.1007/s10517-010-0906-3. PubMed: 21113490.
3. Kostyuk S V, Smirnova T D, Efremova L V, Konkova M S, Alekseeva A Y et al. (2010) Enhanced expression of iNOS in human endothelial cells during long-term culturing with extracellular DNA fragments. Bull Exp Biol Med 149: 191-195. doi: 10.1007/s10517-010-0905-4. PubMed: 21113489.
4. Bulicheva N. Fidelina O, Mkrtumova N, Neverova M, Bogush A et al. (2008) Effect of cell-free DNA of patients with cardiomyopathy and rDNA on the frequency of contraction of electrically paced neonatal rat ventricular myocytes in culture. Annals of the New York Academy of Sciences. Ann N Y Acad Sci 1137: 273-277. doi:10.1196/annals, 1448.023. PubMed: 18837959.
5. Loseva P, Kostyuk S. Malinovskaya E, Clement N, Dechesne C et al. (2012) Extracellular DNA oxidation stimulates activation of NRF2 and reduces the production of ROS in human mesenchymal stem cells. Expert Opin Biol Ther Suppl 1: 85-97.
6. Kostjuk S, Loseva P, Chvartatskaya O, Ershova E, Smirnova T et al. (2012) Extracellular GC-rich DNA activates TLR9- and NF-kB-dependent signaling pathways in human adipose-derived mesenchymal stem cells (haMSCs). Expert Opin Biol Ther Suppl 1:99-111.
7. Kostyuk S V, Tabakov V J, Chestkov V V, Konkova M S, Glebova K V et al. (2013) Oxidized DNA induces an adaptive response in human fibroblasts. Mutat Res: May 1 [Epub ahead of print]. PubMed: 23644378.
8. Ermakov A V, Kostyuk S V, Konkova M S, Egolina N A, Malinovskaya E M et al. (2008) Extracellular DNA fragments. Ann N Y Acad Sci 1137: 41-46. doi: 10.1196/annals, 1448.024. PubMed: 18837923.
9. Ermakov A V, Konkova M S, Kostyuk S V, Egolina N A, Efremova L V et al. (2009) Oxidative stress as a significant factor for development of an adaptive response in irradiated and non-irradiated human lymphocytes after inducing the bystander effect by low-dose X-radiation. Mutat Res 669: 155-161. doi:10.1016/j.mrfmnmm.2009.06.005. PubMed: 19540246.
10. Ermakov A V, Konkova M S, Kostiuk S V, Kalashnikova E A, Kokarovtseva S N et al. (2009) CpG-DNA inhibits cell reactions accompanied with the development of the adaptive response in human lymphocytes after low-dose X-ray exposure. Radiats Biol Radioecol 49: 34-41. PubMed: 19368319.
11. Ermakov A V, Konkova M S, Kostiuk S V, Smirnova T D, Kameneva L V et al. (2010) Bystander effect development in human mesenchymal stem cells after exposure to adaptive dose of X-radiation. Radiats Biol Radioecol 50: 42-51. PubMed: 20297680.
12. Konkova M S, Ermakov A V, Efremova L V, Kostyuk S V, Veiko N N (2010) Influence of X-ray and/or CpG-DNA induced oxidative stress on adaptive response in human lymphocytes. Int J Low Radiat 7: 446-452. doi:10.1504/IJLR.2010.037667.
13. Ermakov A V, Konkova M S, Kostyuk S V, Smirnova T D, Malinovskaya et al. (2011) An extracellular DNA mediated bystander effect produced from low dose irradiated endothelial cells. Mutat Res 712: 1-10. doi: 10.1016/j.mrfmmm.2011.03.002. PubMed: 21392514.
14. Ermakov A V, Konkova M S, Kostyuk S V, Smirnova T D, Efremova L V et al. (2011) Development of the adaptive response and bystander effect induced by low-dose ionizing radiation in human mesenchymal stem cells. In Book “Circulating Nucleic Acids in Plasma and Serum”. Springer Science and Business Media BV: chapter 16.
15. Kostyuk S V. Ermakov A V, Alekseeva A Y, Smirnova T D, Glebova K V et al. (2012) Role of extracellular DNA oxidative modification in radiation induced bystander effects in human endotheliocytes. Mutat Res 729: 52-60. doi:10.1016/j.mrfmmm.2011.09.005. PubMed: 22001237.
16. Glebova K V, Konorova I L, Marakhonov A V, Barskov I V, Khaspekov L G, Veiko N N (2011) Oxidative modification of ecDNA alters its biological action on rat neurons. J Nucleic Acids Investig 2: 28.
17. Fleischhacker M, Schmidt B (2007) Circulating nucleic acids (CNAs) and cancer—a survey. Biochim Biophys Acta 1775: 181-232. PubMed: 17137717.
18. Dawson S J, Tsui D W, Murtaza M, Biggs H, Rueda O M et al. (2013) Analysis of circulating tumor DNA to monitor metastatic breast cancer. N Engl J Med 368: 1199-1209. doi:10.1056/NEJMoa1213261. PubMed: 23484797.
19. Hashad D, Sorour A, Ghazal A, Talaat I (2012) Free circulating tumor DNA as a diagnostic marker for breast cancer. J Clin Lab Anal 26: 467-472. doi: 10.1002/jcla.21548. PubMed: 23143630.
20. Gong B, Xue J, Yu J, Li H, Hu H et al. (2012) Cell-free DNA in blood is a potential diagnostic biomarker of breast cancer. Oncol Lett 3: 897-900. PubMed: 22741014.
21. Agostini M, Pucciarelli S, Enzo M V, Del Bianco P, Briarava M et al. (2011) Circulating cell-free DNA: a promising marker of pathologic tumor response in rectal cancer patients receiving preoperative chemoradiotherapy. Ann Surg Oncol 18: 2461-2468. doi:10.1245/s10434-011-1638-y. PubMed: 21416156.
22. Swystun L L, Mukherjee S, Liaw P C (2011) Breast cancer chemotherapy induces the release of cell-free DNA, a novel procoagulant stimulus. J Thromb Haemost 9: 2313-2321. doi:10.111/j.1538-7836.2011.04465.x. PubMed: 21838758.
23. Roth C, Pantel K, Müller V, Rack B, Kasimir-Bauer S et al. (2011) Apoptosis-related deregulation of proteolytic activities and high serum levels of circulating nucleosomes and DNA in blood correlate with breast cancer progression. BMC Cancer 11: 4. doi: 10.1186/1471-2407-11-4. PubMed: 21211028.
24. Kohler C, Radpour R, Barekati Z, Asadollahi R, Bitzer J et al. (2009) Levels of plasma circulating cell free nuclear and mitochondrial DNA as potential biomarkers for breast tumors. Mol Cancer 8: 105. doi: 10.1186/1476-4598-8-105. PubMed: 19922604.
25. Zhong X Y, Ladewig A, Schmid S, Wight E, Hahn S et al. (2007) Elevated level of cell-free plasma DNA is associated with breast cancer. Arch Gynecol Obstet 276: 327-331. doi: 10.1007/s00404-007-0345-1. PubMed: 17431649.
26. Dumas J J, Merithew E. Sudharshan E, Rajamani D, Hayes S et al. (2001) Multivalent endosome targeting by homodimeric EEA1. Mol Cell 8: 947-958. doi:10.1016/S1097-2765(01)00385-9. PubMed: 11741531.
27. Barber G N (2011) Cytoplasmic DNA innate immune pathways Immunol Rev 243: 99-108. doi:10.1111/j.1600-065X.2011.01051.x. PubMed: 21884170. Oxidized DNA and Genome Instability in Cancer PLOS ONE|www.plosone.org 21 Oct. 2013|Volume 8|Issue 10|e77469
28. Sandholm J, Kauppila J H, Pressey C et al. (2012) Estrogen receptor-α and sex steroid hormones regulate Toll-like receptor-9 expression and invasive function in human breast cancer cells. Breast Cancer Res Treat 132(2): 411-419. doi:10.1007/s10549-011-1590-3. PubMed: 21607583.
29. LeBel C P, Ischiropoulos H, Bondy S C (1992) Evaluation of the probe 2′,7′-dichlorofluorescin as an indicator of reactive oxygen species formation and oxidative stress. Chem Res Toxicol 5: 227-231. doi: 10.1021/tx00026a012. PubMed: 1322737.
30. Altenhöfer S, Kleikers P W, Radermacher K A, Scheurer P, Rob Hermans J J et al. (2012) The NOX toolbox: validating the role of NADPH oxidases in physiology and disease. Cell Mol Life Sci 69: 2327-2343. doi:10.1007/s00018-012-1010-9. PubMed: 22648375.
31. Löbrich M, Shibata A, Beucher A, Fisher A, Ensminger M et al. (2010) gammaH2AX foci analysis for monitoring DNA double-strand break repair: strengths, limitations and optimization. Cell Cycle 9: 662-669. doi:10.4161/cc.9.4.10764. PubMed: 20139725.
32. Fenech M, Kirsch-Volders M, Natarajan A T, Surralles J, Crott J W et al. (2011) Molecular mechanisms of micronucleus, nucleoplasmic bridge and nuclear bud formation in mammalian and human cells. Mutagenesis 26: 125-132. doi: 10.1093/mutage/geq052. PubMed: 21164193.
33. Xu B, Sun Z, Liu Z, Guo H, Liu Q et al. (2011) Replication stress induces micronuclei comprising of aggregated DNA double-strand breaks. PLOS ONE 6: e18618. doi:10.1371/journal.pone.0018618. PubMed: 21525980.
34. Guillaud P, du Manoir S, Seigneurin D (1989) Quantification and topographical description of Ki-67 antibody labelling during the cell cycle of normal fibroblastic (MRC-5) and mammary tumour cell lines (MCF-7). Anal Cell Pathol 1: 25-39. PubMed: 2488698.
35. Naryzhny S N (2008) Proliferating cell nuclear antigen: a proteomics view. Cell Mol Life Sci 65: 3789-3808. doi:10.1007/s00018-008-8305-x. PubMed: 18726183.
36. Nagata S (2005) DNA degradation in development and programmed cell death. Annu Rev Immunol 23: 853-875. doi:10.1146/annurev.immunol.23.021704.115811. PubMed: 15771588.
37. Nguyen T, Sherratt P J, Nioi P, Yang C S, Pickett C B (2005) Nrf2 controls constitutive and inducible expression of ARE-driven genes through a dynamic pathway involving nucleocytoplasmic shuttling by Keap1. J Biol Chem 280: 32485-32492. doi: 10.1074/jbc.M503074200. PubMed: 16000310.
38. Grivennikov S I, Karin M (2010) Dangerous liaisons: STAT3 and NFkappaB collaboration and crosstalk in cancer. Cytokine Growth Factor Rev 21: 11-19. doi: 10.1016/j.cytogfr.2009.11.005. PubMed: 20018552.
39. He G, Karin M (2011) NF-κB and STAT3—key players in liver inflammation and cancer. Cell Res 21: 159-168. doi:10.1038/cr. 2010.183. PubMed: 21187858.
40. Wang D. Baldwin A S Jr (1998) Activation of nuclear factor-kappaB-dependent transcription by tumor necrosis factor-alpha is mediated through phosphorylation of RelA/p65 on serine 529. J Biol Chem 273: 29411-29416. doi: 10.1074/jbc.273.45.29411. PubMed: 9792644.
41. Lee J, Hahm E R, Singh S V (2010) Withaferin A inhibits activation of signal transducer and activator of transcription 3 in human breast cancer cells. Carcinogenesis, 31: 1991-1998. doi: 10.1 093/carcin/bgq175. PubMed: 20724373.
42. Dien J, Amin H M, Chiu N, Wong W et al. (August 2006) Signal transducers and activators of transcription-3 up-regulates tissue inhibitor of etalloproteinase-1 expression and decreases invasiveness of breast cancer. Am J Pathol August; 169: 633-642. doi:10.2353/ajpath.2006.051109. PubMed: 16877361.
43. Gorges T M, Schiller J, Schmitz A, Schuetzmann D, Schatz C et al. (2012) Cancer therapy monitoring in xenografts by quantitative analysis of circulating tumor DNA. Biomarkers 17: 498-506. doi: 10.3109/1354750X.2012.689133. PubMed: 22616911.
44. Mouliere F, Robert B, Arnau Peyrotte E, Del Rio M, Ychou M et al. (2011) High fragmentation characterizes tumour-derived circulating DNA. PLOS ONE 6: e23418. doi:10.1371/journal.pone.0023418. PubMed: 21909401.
45. Henderson P T, Evans M D, Cooke M S (2010) Salvage of oxidized guanine derivatives in the (2′-deoxy)ribonucleotide pool as source of mutations in DNA. Mutat Res 703: 11-17. doi:10.1016/j.mrgentox.2010.08.021. PubMed: 20833264.
46. Kamiya H (2010) Mutagenicity of oxidized DNA precursors in living cells: Roles of nucleotide pool sanitization and DNA repair enzymes, and translesion synthesis DNA polymerases. Mutat Res 703: 32-36. doi:10.1016/j.mrgentox.2010.06.003. PubMed: 20542139.
47. Pedruzzi L M, Stockler-Pinto M B, Leite M Jr, Mafra D (2012) Nrf2-keap1 system versus NF-κB: the good and the evil in chronic kidney disease? Biochimie 94: 2461-2366. doi:10.1016/j.biochi.2012.07.015. PubMed: 22874821.48. Wakabayashi N, Slocum S L, Skoko J J, Shin S, Kensler T W (2010) When NRF2 talks, who's listening? Antioxid Redox Signal 13: 1649-1663. doi:10.1089/ars.2010.3216. PubMed: 20367496.
49. Handy D E, Loscalzo J (2012) Redox regulation of mitochondrial function. Antioxid Redox Signal 16: 1323-1367. doi:10.1089/ars.2011.4123. PubMed: 22146081.
50. Vera-Ramirez L, Ramirez-Tortosa M, Perez-Lopez P, Granados-Principal S. Battino M, Quiles J L, (2012) Long-term effects of systemic cancer treatment on DNA oxidative damage: the potential for targeted therapies. Cancer Lett 327: 134-141. doi: 10.1016/j.canlet.2011.12.029. PubMed: 22274413.
51. Szatrowski T P, Nathan C F (1991) Production of large amounts of hydrogen peroxide by human tumor cells. Cancer Res 51: 794-798. PubMed: 1846317.
52. Toyokuni S, Okamoto K, Yodoi J, Hiai H (1995) Persistent oxidative stress in cancer. FEBS Lett 358: 1-3. doi:10.1016/0014-5793(94)01368-B. PubMed: 7821417.
53. Iwasa Y, Nowak M A, Michor F (2006) Evolution of resistance during clonal expansion. Genetics 172: 2557-2566. PubMed: 16636113.
54. Crujeiras A B, Diaz-Lagares A, Carreira M C, Amil M, Casanueva F F (2013) Oxidative stress associated to dysfunctional adipose tissue: a potential link between obesity, type 2 diabetes mellitus and breast cancer. Free Radic Res 47: 243-256. doi: 10.3109/10715762.2013.772604. PubMed: 23409968.
M. Ojima, H. Eto, N. Ban, and M. Kai, “Radiation-induced bystander effects induce radioadaptive response by low-dose radiation,” Radiation Protection Dosimetry, vol. 146, no. 1-3, pp. 276-279, 2011.
F. Ballarini, M. Biaggi, A. Ottolenghi, and O. Sapora, “Cellular communication and bystander effects: a critical review for modelling low-dose radiation action,” Mutation Research, vol. 501, no. 1-2, pp. 1-12, 2002.
C. Mothersill and C. Seymour, “Radiation-induced bystander effects and adaptive responses—the Yin and Yang of low dose radiobiology?” Mutation Research, vol. 568, no. 1, pp. 121-128, 2004.
B. R. Scott, “A biological-based model that links genomic instability, bystander effects, and adaptive response,” Mutation Research, vol. 568, no. 1, pp. 129-143, 2004.
M. Ko, X. Y. Lao, R. Kapadia, E. Elmore, and J. L. Redpath, “Neoplastic transformation in vitro by low doses of ionizing radiation: role of adaptive response and bystander effects,” Mutation Research, vol. 597, no. 1-2, pp. 11-17, 2006.
R. Iyer and B. E. Lehnert, “Low dose, low-LET ionizing radiation-induced radioadaptation and associated early responses in unirradiated cells,” Mutation Research, vol. 503, no. 1-2, pp. 1-9, 2002.
S. A. Mitchell, S. A. Marino, D. J. Brenner, and E. J. Hall, “Bystander effect and adaptive response in C3H 10T1/2 cells,” International Journal of Radiation Biology, vol. 80, no. 7, pp. 465-472, 2004.
H. Nagasawa and J. B. Little, “Induction of sister chromatid exchanges by extremely low doses of α-particles,” Cancer Research, vol. 52, no. 22, pp. 6394-6396, 1992.
C. Mothersill and C. B. Seymour, “Cell-cell contact during gamma irradiation is not required to induce a bystander effect in normal human keratinocytes: evidence for release during irradiation of a signal controlling survival into the medium,” Radiation Research, vol. 149, no. 3, pp. 256-262, 1998.
E. I. Azzam, S. M. de Toledo, and J. B. Little, “Stress signaling from irradiated to non-irradiated cells,” Current Cancer Drug Targets, vol. 4, no. 1, pp. 53-64, 2004.
H. Matsumoto, A. Takahashi, and T. Ohnishi, “Radiation-induced adaptive responses and bystander effects,” Uchu Seibutsu Kagaku, vol. 18, no. 4, pp. 247-254, 2004.
S. M. de Toledo and E. I. Azzam, “Adaptive and bystander responses in human and rodent cell cultures exposed to low level ionizing radiation: the impact of linear energy transfer,” Dose Response, vol. 4, no. 4, pp. 291-301, 2006.
T. K. Hei, H. Zhou, V. N. Ivanov et al., “Mechanism of radiation induced bystander effects: a unifying model,” Journal of Pharmacy and Pharmacology, vol. 60, no. 8, pp. 943-950, 2008.
J. Rzeszowska-Wolny, W. M. Przybyszewski, and M. Widel, “Ionizing radiation-induced bystander effects, potential targets for modulation of radiotherapy,” European Journal of Pharmacology, vol. 625, no. 1-3, pp. 156-164, 2009.
K. M. Prise and J. M. O'Sullivan, “Radiation-induced bystander signalling in cancer therapy,” Nature Reviews Cancer, vol. 9, no. 5, pp. 351-360, 2009.
S. Sjostedt and E. Bezak, “Non-targeted effects of ionising radiation and radiotherapy,” Australasian Physical and Engineering Sciences in Medicine, vol. 33, no. 3, pp. 219-231, 2010.
B. J. Blyth and P. J. Sykes, “Radiation-induced bystander effects: what are they, and how relevant are they to human radiation exposures?” Radiation Research, vol. 176, no. 2, pp. 139-157, 2011.
D. M. Spitkovskii, N. N. Veiko, A. V. Ermakov et al., “Structural and functional changing induced by exposure to adaptive doses of X-rays in the human lymphocytes both normal and defective by reparation of DNA double strands breaks,” Radiation Biology, Radioecology, vol. 43, no. 2, pp. 136-143, 2003.
N. N. Veiko, A. V. Ermakov, N. A. Egolina et al., “Activation of total and ribosomal RNA transcription under adapting doses of ionizing radiation inducing displacement of chromosome loci in human Go-lymphocyte,” Radiatsionnaya Biologiya. Radioekologiya, vol. 44, no. 5, pp. 501-508, 2004.
A. V. Ermakov, S. V. Kostiuk, N. A. Egolina, E. M. Malinovskaia, N. N. Veiko, and D. M. Spitkovskii, “The DNA fragments obtained from the culture media exposed to adaptive doses of the ionizing radiation as factors of stress signaling between lymphocytes and bystander cells,” Radiatsionnaia Biologiia, Radioecologiia, vol. 47, no. 2, pp. 133-140, 2007.
S. V. Kostiuk, I. A. Zamulaeva, R. K. Agapova et al., “The changing of cell-free DNA properties of peripheral blood and TCR-mutant cell frequency in individuals exposed to ionizing radiation,” Radiatsionnaia Biologiia, Radioecologiia, vol. 48, no. 1, pp. 5-13, 2008.
A. V. Ermakov, S. V. Kostyuk, M. S. Konkova, N. A. Egolina E. M. Malinovskaya, and N. N. Veiko, “Extracellular DNA fragments: factors of stress signaling between X-irradiated and nonirradiated human lymphocytes,” Annals of the New York Academy of Sciences, vol. 1137, pp. 41-46, 2008.
A. V. Ermakov, M. S. Konkova, S. V. Kostyuk, N. A. Egolina, L. V. Efremova, and N. N. Veiko, “Oxidative stress as a significant factor for development of an adaptive response in irradiated and nonirradiated human lymphocytes after inducing the bystander effect by low-dose X-radiation,” Mutation Research, vol. 669, no. 1-2, pp. 155-161, 2009.
A. V. Ermakov, M. S. Konkova, S. V. Kostiuk et al., “CpG-DNA inhibits cell reactions accompanied with the development of the adaptive response in human lymphocytes after low-dose X-ray exposure,” Radiation Biology, Radioecology, vol. 49, no. 1, pp. 34-41, 2009.
A. V. Ermakov, M. S. Konkova, S. V. Kostiuk et al., “The response of human cancer stem cells on low-dose X-ray exposure,” Radiatsionnaia Biologiia, Radioecologiia, vol. 49, no. 5, pp. 528-537, 2009.
A. V. Ermakov, M. S. Konkova, S. V. Kostiuk et al., “Bystander effect development in human mesenchymal stem cells after exposure to adaptive dose of X-radiation,” Radiatsionnaia Biologiia, Radioccologiia, vol. 50, no. 1, pp. 42-51, 2010.
M. S. Konkova, A. V. Ermakov, L. V. Efremova, S. V. Kostyuk, and N. N. Veiko, “Influence of X-ray and/or CpG-DNA induced oxidative stress on adaptive response in human lymphocytes,” International Journal of Low Radiation, vol. 7, no. 6, pp. 446-452, 2010.
L. V. Efremova, A. Y. Alekseeva, M. S. Konkova et al., “Extracellular DNA affects NO content in human endothelial cells,” Bulletin of Experimental Biology and Medicine, vol. 149, no. 2, pp. 196-200, 2010.
S. V. Kostyuk, T. D. Smirnova, L. V. Efremova et al., “Enhanced expression of iNOS in human endothelial cells during long term culturing with extracellular DNA fragments,” Bulletin of Experimental Biology and Medicine, vol. 149, no. 2, pp. 191-195, 2010.
A. V. Ermakov, M. S. Konkova, S. V. Kostyuk et al., “An extracellular DNA mediated bystander effect produced from low dose irradiated endothelial cells,” Mutation Research, vol. 712, no. 1-2, pp. 1-10, 2011.
A. V. Ermakov, M. S. Konkova, S. V. Kostyuk et al., “Development of the adaptive response and bystander effect induced by low-dose ionizing radiation in human mesenchymal stem cells,” in Proceedings of the 6th International Conference on Circulating Nucleic Acids in Plasma and Serum (CNAPS '11), pp. 225-231, 2011.
K. V. Glebova, I. L. Konorova, A. V. Marakhonov, I. V. Barskov, L. G. Khaspekov, and N. N. Veiko, “Oxidative modification of ecDNA alter its biological action on rat neurons,” Journal of Nucleic Acids Investigation, vol. 2, no. 1, p. 28, 2011.
P. Loseva, S. Kostyuk, E. Malinovskaya et al., “Extracellular DNA oxidation stin-julates activation of NRF2 and reduces the production of ROS in human mesenchymal stem cells,” Expert Opinion on Biological Therapy, vol. 12, supplement 1, pp. 85-97, 2012.
S. V. Kostyuk, A. V. Ermakov, A. Y. Alekseeva et al., “Role of extracellular DNA oxidative modification in radiation induced bystander effects in human endotheliocytes,” Mutation Research, vol. 729, no. 1-2, pp. 52-60, 2012.
S. Kostjuk, P. Loseva, O. Chvartatskaya et al., “Extracellular GC-rich DNA activates TLR9- and NF-κB-dependent signaling pathways in human adipose-derived mesenchymal stem cells (haMSCs),” Expert Opinion on Biological Therapy, vol. 12, supplement 1, pp. 99-111, 2012.
M. T. Russo, M. F. Blasi, F. Chiera et al., “The oxidized deoxynucleoside triphosphate pool is a significant contributor to genetic instability in mismatch repair-deficient cells,” Molecular and Cellular Biology, vol. 24, no. 1, pp. 465-474, 2004.
E. Speina, K. D. Arczewska, D. Gackowski et al., “Contribution of hMTH1 to the maintenance of 8-oxoguanine levels in lung DNA of non-small-cell lung cancer patients,” Journal of the National Cancer Institute, vol. 97, no. 5, pp. 384-395, 2005.
D. Mangal, D. Vudathala, J. Park, H. L. Seon, T. M. Penning, and I. A. Blair, “Analysis of 7,8-dihydro-8-oxo-2′-deoxyguanosine Oxidative Medicine and Cellular Longevity 9 in cellular DNA during oxidative stress,” Chemical Research in Toxicology, vol. 22, no. 5, pp. 788-797, 2009.
J. Ravanat, T. Douki, P. Duez et al., “Cellular background level of 8-oxo-7,8-dihydro-2′-deoxyguanosine: an isotope based method to evaluate artefactual oxidation of DNA during its extraction and subsequent work-up,” Carcinogenesis, vol. 23, no. 11, pp. 1911-1918, 2002.
D. Li, W. Zhang, J. Zhu et al., “Oxidative DNA damage and 8-hydroxy-2-deoxyguanosine DNA glycosylase/apurinic lyase in human breast cancer,” Molecular Carcinogenesis, vol. 31, no. 4, pp. 214-223, 2001.
E. S. Hwang and P. E. Bowen, “DNA damage, a biomarker of carcinogenesis: its measurement and modulation by diet and environment,” Critical Reviews in Food Science and Nutrition, vol. 47, no. 1, pp. 27-50, 2007.
A. Valavanidis. T. Vlachogianni, and C. Fiotakis, “8-hydroxy-2′-deoxyguanosine (8-OHdG): a critical biomarker of oxidative stress and carcinogenesis,” Journal of Environmental Science and Health C, Environmental Carcinogenesis & Ecotoxicology=Reviews, vol. 27, no. 2, pp. 120-139, 2009.
S. Loft, P. Møller, M. S. Cooke, R. Rozalski, and R. Olinski, “Antioxidant vitamins and cancer risk: is oxidative damage to DNA a relevant biomarker?” European Journal of Nutrition, vol. 47, no. 2, pp. 19-28, 2008.
D. C. Malins and R. Haimanot, “Major alterations in the nucleotide structure of DNA in cancer of the female breast,” Cancer Research, vol. 51, no. 19, pp. 5430-5432, 1991.
A. Matsui, T. Ikeda, K. Enomoto et al., “Increased formation of oxidative DNA damage, 8-hydroxy-2′-deoxyguanosine, in human breast cancer tissue and its relationship to GSTP1 and COMT genotypes,” Cancer Letters, vol. 151, no. 1, pp. 87-95, 2000.
J. Musarrat. J. Arezina-Wilson, and A. A. Wani, “Prognostic and aetiological relevance of 8-hydroxyguanosine in human breast carcinogenesis,” European Journal of Cancer A, vol. 32, no. 7, pp. 1209-1214, 1996.
E. Gajewski, S. Gaur, S. A. Akman, L. Matsumoto, J. N. A. van Balgooy, and J. H. Doroshow, “Oxidative DNA base damage in MCF-10A breast epithelial cells at clinically achievable concentrations of doxorubicin,” Biochemical Pharmacology, vol. 73, no. 12, pp. 1947-1956, 2007.
D. C. Malins, P. M. Johnson, T. M. Wheeler et al., “Age-related radical-induced DNA damage is linked to prostate cancer,” Cancer Research, vol. 61, no. 16, pp. 6025-6028, 2001.
D. C. Malins, P. M. Johnson, E. A. Barker, N. L. Polissar, T. M. Wheeler, and K. M. Anderson, “Cancer-related changes in prostate DNA as men age and early identification of metastasis in primary prostate tumors,” Proceedings of the National Academy of Sciences of the United States of America, vol. 100, no. 9, pp. 5401-5406, 2003.
S. Pathak, R. Singh, R. D. Verschoyle et al., “Androgen manipulation alters oxidative DNA adduct levels in androgen-sensitive prostate cancer cells grown in vitro and in vivo.” Cancer Letters, vol. 261, no. 1, pp. 74-83, 2008.
M. Sanchez, J. V. Torres, C. Tormos et al., “Impairment of antioxidant enzymes, lipid peroxidation and 8-oxo-2′-deoxyguanosine in advanced epithelial ovarian carcinoma of a Spanish-community,” Cancer Letters, vol. 233, no. 1, pp. 28-35, 2006.
M. Foksinski, R. Kotzbach, W. Szymanski, and R. Olinski, “The level of typical biomarker of oxidative stress 8-hydroxy-2′-deoxyguanosine is higher in uterine myomas than in control tissues and correlates with the size of the tumor,” Free Radical Biology and Medicine, vol. 29, no. 7, pp. 597-601, 2000.
P. Jaruga, T. H. Zastawny, J. Skokowski, M. Dizdaroglu, and R. Olinski, “Oxidative DNA base damage and antioxidant enzyme activities in human lung cancer,” The FEBS Letters, vol. 341, no. 1, pp. 59-64, 1994.
R. Olinski, T. Zastawny, J. Budzbon, J. Skokowski, W. Zegarski, and M. Dizdaroglu, “DNA base modifications in chromatin of human cancerous tissues,” The FEBS Letters, vol. 309, no. 2, pp. 193-198, 1992.
K. Roszkowski, W. Jozwicki, P. Blaszczyk, A. Mucha-Malecka, and A. Siomek, “Oxidative damage DNA: 8-oxogua and 8-oxodG as molecular markers of cancer,” Medical Science Monitor, vol. 17, no. 6, pp. CR329-CR333, 2011.
S. Lagadu, M. Lechevrel, F. Sichel et al., “8-oxo-7,8-dihydro-2′-deoxyguanosine as a biomarker of oxidative damage in oesophageal cancer patients: lack of association with antioxidant vitamins and polymorphism of hOGG1 and GST,” Journal of Experimental and Clinical Cancer Research, vol. 29, no. 1, pp. 157-167, 2010.
A. Szaflarska-Poplawska, A. Siomek, M. Czerwionka-Szaflarska et al., “Oxidatively damaged DNA/oxidative stress in children with celiac disease,” Cancer Epidemiology Biomarkers and Prevention, vol. 19, no. 8, pp. 1960-1965, 2010.
R. Gnana Oli, G. Fazeli, W. Kuhn, S. Walitza, M. Gerlach, and H. Stopper, “No increased chromosomal damage in 1-DOPA treated patients with Parkinson's disease: a pilot study,” Journal of Neural Transmission, vol. 117, no. 6, pp. 737-746, 2010.
T. Dziaman, T. Huzarski, D. Gackowski et al., “Selenium supplementation reduced oxidative DNA damage in adnexectomized BRCA1 mutations carriers,” Cancer Epidemiology Biomarkers and Prevention, vol. 18, no. 11, pp. 2923-2928, 2009.
J. Guz, M. Foksinski, A. Siomek et al., “The relationship between 8-oxo-7,8-dihydro-2′-deoxyguanosine level and extent of cytosine methylation in leukocytes DNA of healthy subjects and in patients with colon adenomas and carcinomas,” Mutation Research, vol. 640, no. 1-2, pp. 170-173, 2008.
S. V. Vulimiri, X. Wu, W. Baer-Dubowska, M. de Andrade et al., “Analysis of aromatic DNA adducts and 7,8-dihydro-8-oxo-2′-deoxyguanosine in lymphocyte DNA from a case-control study of lung cancer involving minority populations,” Molecular Carcinogenesis, vol. 27, no. 1, pp. 34-46, 2000.
M. Yen, S. Kao, A. Wang, and Y. Wei, “Increased 8-hydroxy-2□-deoxyguanosine in leukocyte DNA in Leber's hereditary optic neuropathy,” Investigative Ophthalmology and Visual Science, vol. 45, no. 6, pp. 1688-1691, 2004.
A. Kikuchi, A. Takeda, H. Onodera et al., “Systemic increase of oxidative nucleic acid damage in Parkinson's disease and multiple system atrophy,” Neurobiology of Disease, vol. 9, no. 2, pp. 244-248, 2002.
L. Haider, M. T. Fischer, J. M. Frischer et al., “Oxidative damage in multiple sclerosis lesions,” Brain, vol. 134, no. 7, pp. 1914-1924, 2011.
L. Lyras, N. J. Calms, A. Jenner, P. Jenner, and B. Halliwell, “An assessment of oxidative damage to proteins, lipids, and DNA in brain from patients with Alzheimer's disease,” Journal of Neurochemistry, vol. 68, no. 5, pp. 2061-2069, 1997.
N. Nishioka and S. E. Arnold, “Evidence for oxidative DNA damage in the hippocampus of elderly patients with chronic schizophrenia,” The American Journal of Geriatric Psychiatry, vol. 12, no. 2, pp. 167-175, 2004.
D. Tarng, T. Huang, Y. Wei et al., “8-Hydroxy-2′-deoxyguanosine of leukocyte DNA as a marker of oxidative stress in chronic hemodialysis patients,” American Journal of Kidney Diseases, vol. 36, no. 5, pp. 934-944, 2000.
D. C. Tarng, T. P. Huang, T. Y. Liu et al., “Effect of vitamin E-bonded membrane on the 8-hydroxy-2′-deoxyguanosine level in leukocyte DNA of hemodialysis patients,” Kidney International, vol. 58, no. 2, pp. 790-799, 2000.
D. Tarng, T. W. Chen, T. Huang, C. Chen, T. Liu, and Y. Wei, “Increased oxidative damage to peripheral blood leukocyte DNA in chronic peritoneal dialysis patients,” Journal of the American Society of Nephrology, vol. 13, no. 5, pp. 1321-1330, 2002.
G. Pagano, P. Degan, M. d'Ischia et al., “Gender- and age-related distinctions for the in vivo pro-oxidant state in Fanconi anaemia patients,” Carcinogenesis, vol. 25, no. 10, pp. 1899-1909, 2004.
I. M. Agbaje, C. M. McVicar, B. C. Schock et al., “Increased concentrations of the oxidative DNA adduct 7,8-dihydro-8-oxo-2-deoxyguanosine in the germ-line of men with type 1 diabetes,” Reproductive BioMedicine Online, vol. 16, no. 3, pp. 401-409, 2008.
S. Yurdakul, B. Ozben, A. K. Bilge, U. M. Turkoglu, S. Arkaya, and Y. Nisanci, “Oxidative DNA damage is significantly correlated with flow-mediated dilation in patients with coronary artery disease,” Journal of Investigative Medicine, vol. 56, no. 7, pp. 925-930, 2008.
M. Satoh, Y. Minami, Y. Takahashi, T. Tabuchi, T. Itoh, and M. Nakamura, “Effect of intensive lipid-lowering therapy on telomere erosion in endothelial progenitor cells obtained from patients with coronary artery disease,” Clinical Science, vol. 116, no. 11-12, pp. 827-835, 2009.
T. Kaneko, S. Tahara, and M. Matsuo, “Non-linear accumulation of 8-hydroxy-2′-deoxyguanosine, a marker of oxidized DNA damage, during aging,” Mutation Research, vol. 316, no. 5-6, pp. 277-285, 1996.
T. Kaneko, S. Tahara, and M. Matsuo, “Retarding effect of dietary restriction on the accumulation of 8-hydroxy-2′-deoxyguanosine in organs of Fischer 344 rats during aging,” Free Radical Biology and Medicine, vol. 23, no. 1, pp. 76-81, 1997.
C. G. Fraga, M. K. Shigenaga, J. W. Park, P. Degan, and B. N. Ames, “Oxidative damage to DNA during aging: 8-hydroxy-2′-deoxyguanosine in rat organ DNA and urine,” Proceedings of the National Academy of Sciences of the United States of America, vol. 87, no. 12, pp. 4533-4537, 1990.
A. Siomek, D. Gackowski, R. Rozalski et al., “Higher leukocyte 8-oxo-7,8-dihydro-2′-deoxyguanosine and lower plasma ascorbate in aging humans?” Antioxidants and Redox Signaling, vol. 9, no. 1, pp. 143-150, 2007.
T. Kaneko, S. Tahara, T. Taguchi, and H. Kondo, “Accumulation of oxidative DNA damage, 8-oxo-2′-deoxyguanosine, and change of repair systems during in vitro cellular aging of cultured human skin fibroblasts,” Mutation Research, vol. 487, no. 1-2, pp. 19-30, 2001.
Y. Homma, M. Tsunoda, and H. Kasai, “Evidence for the accumulation of oxidative stress during cellular ageing of human diploid fibroblasts.” Biochemical and Biophysical Research Communications, vol. 203, no. 2, pp. 1063-1068, 1994.
S. Hajizadeh, J. DeGroot, J. M. TeKoppele, A. Tarkowski, and L. V. Collins, “Extracellular mitochondrial DNA and oxidatively damaged DNA in synovial fluid of patients with rheumatoid arthritis,” Arthritis Research & Therapy, vol. 5, no. 5, pp. R234-R240, 2003.
B. Zhang, A. Angelidou, K. D. Alysandratos et al., “Mitochondrial DNA and anti-mitochondrial antibodies in serum of autistic children,” Journal of Neuroinflammation, vol. 7, pp. 80-85, 2010.
A. Cossarizza, M. Pinti, M. Nasi et al., “Increased plasma levels of extracellular mitochondrial DNA during HIV infection: a new role for mitochondrial damage-associated molecular patterns during inflammation,” Mitochondrion, vol. 11, no. 5, pp. 750-755, 2011.
E. M. Malinovskaya, T. D. Smirnova, N. A. Egolina et al., “Changes in human ribosomal genes ensemble with ageing,” Medical Genetics, vol. 7, no. 2, pp. 10-16, 2008.
M. Fleischhacker and B. Schmidt, “Circulating nucleic acids (CNAs) and cancer—a survey,” Biochimica et Biophysica Acta, vol. 1775, no. 1, pp. 181-232, 2007.
S. Jahr, H. Hentze, S. Englisch et al., “DNA fragments in the blood plasma of cancer patients: quantitations and evidence for their origin from apoptotic and necrotic cells,” Cancer Research, vol. 61, no. 4, pp. 1659-1665, 2001.
M. van derVaart and P. J. Pretorius, “Circulating DNA: its origin and fluctuation,” Annals of the New York Academy of Sciences, vol. 1137, pp. 18-26, 2008.
G. Sozzi, D. Conte, L. Mariani et al., “Analysis of circulating tumor DNA in plasma at diagnosis and during follow-up of lung cancer patients,” Cancer Research, vol. 61, no. 12, pp. 4675-4678, 2001.
J. Jylhava, M. Jylha, T. Lehtimaki, A. Hervonen, and M. Hurme, “Circulating cell-free DNA is associated with mortality and inflammatory markers in nonagenarians: the Vitality 90+ Study,” Experimental Gerontology, vol. 47, no. 5, pp. 372-378, 2012.
J. Atamaniuk, C. Vidotto, M. Kinzlbauer, N. Bachl, B. Tiran, and H. Tschan, “Cell-free plasma DNA and purine nucleotide degradation markers following weightlifting exercise,” European Journal of Applied Physiology, vol. 110, no. 4, pp. 695-701, 2010.
S. Hahn, C. Rusterholz, I. Hosli, and O. Lapaire, “Cell-free nucleic acids as potential markers for preeclampsia,” Placenta, vol. 32, no. 1, pp. S17-S20, 2011.
I. L. Konorova and N. N. Veiko, “Emotional stress in rats changes concentration and composition of extracellular DNA circulating in blood plasma under normal conditions and in cerebral ischemia,” Bulletin of Experimental Biology and Medicine, vol. 153, no. 3, pp. 305-308, 2012.
N. N. Veiko, N. V. Bulycheva, O. A. Roginko et al., “Ribosomal repeat in cell free DNA as a marker for cell death,” Biochemistry (Moscow) Supplement Series B: Biomedical Chemistry, vol. 2, no. 2, pp. 198-207, 2008.
C. Wang, N. Yang, C. Chang, S. Liou, and H. Lee, “Rapid and simple one-step membrane extraction for the determination of 8-hydroxy-2′-deoxyguanosine in human plasma by a combination of on-line solid phase extraction and LC-MS/MS,” Journal of Chromatography B: Analytical Technologies in the Biomedical and Life Sciences, vol. 879, no. 30, pp. 3538-3543, 2011.
C. Hu, Y. Huang, Y. Li, and M. Chao, “Correlation between concentrations of 8-oxo-7,8-dihydro-2□-deoxyguanosine in urine, plasma and saliva measured by on-line solid-phase extraction LC-MS/MS,” Clinica Chimica Acta, vol. 411, no. 17-18, pp. 1218-1222, 2010.
R. A. El-Zein, C. M. Monroy, A. Cortes, M. R. Spitz, A. Greisinger, and C. J. Etzel. “Rapid method for determination of DNA repair capacity in human peripheral blood lymphocytes amongst smokers,” BMC Cancer, vol. 10, pp. 439-448, 2010.
C. S. Shin, B. S. Moon, K. S. Park et al., “Serum 8-hydroxyguanine levels are increased in diabetic patients,” Diabetes Care, vol. 24, no. 4, pp. 733-737, 2001.
Z. Hamurcu, F. Bayram, G. Kahriman, H. Donmez-Altuntas, and G. Baskol, “Micronucleus frequency in lymphocytes and 8-hydroxydeoxyguanosine level in plasma of women with polycystic ovary syndrome,” Gynecological Endocrinology, vol. 26, no. 8, pp. 590-595, 2010.
H. Pan, H. Zhang, D. Chang, H. Li, and H. Sui, “The change of oxidative stress products in diabetes mellitus and diabetic retinopathy,” British Journal of Ophthalmology, vol. 92, no. 4, pp. 548-551, 2008.
R. J. Bloomer and K. H. Fisher-Wellman, “Blood oxidative stress biomarkers: influence of sex, exercise training status, and dietary intake,” Gender Medicine, vol. 5, no. 3, pp. 218-228, 2008.
M. S. Cooke, R. Singh, G. K. Hall et al., “Evaluation of enzyme-linked immunosorbent assay and liquid chromatography tandem mass spectrometry methodology for the analysis of 8-oxo-7,8-dihydro-2′-deoxyguanosine in saliva and urine,” Free Radical Biology and Medicine, vol. 41, no. 12, pp. 1829-1836, 2006.
D. Chang, Q. Sha, X. Zhang et al., “The evaluation of the oxidative stress parameters in patients with primary angle closure glaucoma,” PLoS ONE, vol. 6, no. 11, Article ID e27218, 2011.
E. M. Park, M. K. Shigenaga, P. Degan et al., “Assay of excised oxidative DNA lesions: isolation of 8-oxoguanine and its nucleoside derivatives from biological fluids with a monoclonal antibody column,” Proceedings of the National Academy of Sciences of the United States of America, vol. 89, no. 8, pp. 3375-3379, 1992.
M. B. Bogdanov, M. F. Beal, D. R. McCabe, R. M. Griffin, and W. R. Matson, “A carbon column-based liquid chromatography electrochemical approach to routine 8-hydroxy-2′-deoxyguanosine measurements in urine and other biologic matrices: a one-year evaluation of methods,” Free Radical Biology and Medicine, vol. 27, no. 5-6, pp. 647-666, 1999.
N. N. Veiko, N. O. Shubaeva, S. M. Ivanova, A. I. Speranskii, N. A. Lyapunova, and D. M. Spitkovskii, “Blood serum DNA in patients with rheumatoid arthritis is considerably enriched with fragments of ribosomal repeats containing immunostimulatory CpG-motifs,” Bulletin of Experimental Biology and Medicine, vol. 142, no. 3, pp. 313-316, 2006.
N. N. Veiko and D. M. Spitkovskii, “The accumulation of single-stranded breaks does not lead to paired DNA damage—the characteristic of the transcribing fragment of the human ribosomal operon that allows its being detected in biological fluids at the death of different body cells,” Radiation Biology, Radioecology, vol. 40, no. 4, pp. 396-404, 2000.
E. S. Morozkin, E. M. Loseva, I. V. Morozov et al., “A comparative study of cell-free apoptotic and genomic DNA using FISH and massive parallel sequencing,” Expert Opinion on Biological Therapy, vol. 12, supplement 1, pp. 11-17, 2012.
M. Suter and C. Richter, “Fragmented mitochondrial DNA is the predominant carder of oxidized DNA bases,” Biochemistry, vol. 38, no. 1, pp. 459-464, 1999.
N. Bulicheva, O. Fidelina, N. Mkrtumova et al., “Effect of cell-free DNA of patients with cardiomyopathy and rDNA on the frequency of contraction of electrically paced neonatal rat ventricular myocytes in culture,” Annals of the New York Academy of Sciences, vol. 1137, pp. 273-277, 2008.
H. Yoshida, M. Nishikawa, T. Kiyota, H. Toyota, and Y. Takakura, “Increase in CpG DNA-induced inflammatory responses by DNA oxidation in macrophages and mice,” Free Radical Biology and Medicine, vol. 51, no. 2, pp. 424-431, 2011.
H. Ahsan, A. Ali, and R. Ali, “Oxygen free radicals and systemic autoimmunity,” Clinical and Experimental Immunology, vol. 131, no. 3, pp. 398-404, 2003.
M. S. Cooke, K. E. Herbert, P. C. Butler, and J. Lunec, “Further evidence for a possible role of conformation in the immunogenicity and antigenicity of the oxidative DNA lesion, 8-oxo-2′-deoxyguanosine,” Free Radical Research, vol. 28, no. 5, pp. 459-469, 1998.
A. V. Ermakov, N. I. Pospekhova, and D. M. Spitkovskii, “Subpopulation of lymphocytes in peripheral human blood responds to the action of low doses of ionizing radiation and interleukine-2 and also to the action of both factors,” Radiatsionnaya Biologiya. Radioekologiya, vol. 40, no. 1, pp. 62-70, 2000.
W. M. Bonner, “Low-dose radiation: thresholds, bystander effects, and adaptive responses,” Proceedings of the National Academy of Sciences of the United States of America, vol. 100, no. 9, pp. 4973-4975, 2003.
W. F. Morgan, A. Hartmann, C. L. Limoli, S. Nagar, and B. Ponnaiya, “Bystander effects in radiation-induced genomic instability,” Mutation Research, vol. 504, no. 1-2, pp. 91-1100, 2002.
J. K. Leach, G. van Tuyle, P. S. Lin, R. Schmidt-Ullrich, and R. B. Mikkelsen, “Ionizing radiation-induced, mitochondria dependent generation of reactive oxygen/nitrogen,” Cancer Research, vol. 61, no. 10, pp. 3894-3901, 2001.
M. Dizdaroglu, “Oxidative damage to DNA in mammalian chromatin,” Mutation Research, vol. 275, no. 3-6, pp. 331-342, 1992.
A. P. Breen and J. A. Murphy, “Reactions of oxyl radicals with DNA,” Free Radical Biology and Medicine, vol. 18, no. 6, pp. 1033-1077, 1995.
J. Cadet, T. Delatour, T. Douki et al., “Hydroxyl radicals and DNA base damage,” Mutation Research, vol. 424, no. 1-2, pp. 9-21, 1999.
J. Cadet, T. Douki, D. Gasparutto, and J. Ravanat, “Oxidative damage to DNA: formation, measurement and biochemical features,” Mutation Research, vol. 531, no. 1-2, pp. 5-23, 2003.
S. Burney, J. L. Caulfield, J. C. Niles, J. S. Wishnok, and S. R. Tannenbaum, “The chemistry of DNA damage from nitric oxide and peroxynitrite,” Mutation Research, vol. 424, no. 1-2, pp. 37-49, 1999.
S. Kawanishi, Y. Hiraku, and S. Oikawa, “Mechanism of guanine-specific DNA damage by oxidative stress and its role in carcinogenesis and aging,” Mutation Research, vol. 488, no. 1, pp. 65-76, 2001.
C. Chatgilialoglu and P. O.'Neill, “Free radicals associated with DNA damage,” Experimental Gerontology, vol. 36, no. 9, pp. 1459-1471, 2001.
N. Hamada, G. Schettino, G. Kashino et al., “Histone H2AX phosphorylation in normal human cells irradiated with focused ultrasoft X rays: evidence for chromatin movement during repair,” Radiation Research, vol. 166, no. 1, part 1, pp. 31-38, 2006.
M. Gandhi, V. N. Evdokimova, K. T. Cuenco et al., “Homologous chromosomes make contact at the sites of double-strand breaks in genes in somatic G0/G1-phase human cells,” 12 Oxidative Medicine and Cellular Longevity Proceedings of the National Academy of Sciences of the United States of America, vol. 109, no. 24, pp. 9454-9459, 2012.
F. M. Lyng, C. B. Seymour, and C. Mothersill, “Early events in the apoptotic cascade initiated in cells treated with medium from the progeny of irradiated cells,” Radiation Protection Dosimetry, vol. 99, no. 1-4, pp. 169-172, 2002.
G. Hartmann and A. M. Krieg, “Mechanism and function of a newly identified CpG DNA motif in human primary B cells,” The Journal of Immunology, vol. 164, no. 2, pp. 944-952, 2000.
L. J'ozsef, T. Khreiss, D. El Kebir, and J. G. Filep, “Activation of TLR-9 induces IL-8 secretion through peroxynitrite signaling in human neutrophils,” The Journal of Immunology, vol. 176, no. 2, pp. 1195-1202, 2006.
J. A. Pedras-Vasconcelos, D. Goucher, M. Puig et al., “CpG oligodeoxynucleotides protect newborn mice from a lethal challenge with the neurotropic Tacaribe arenavirus,” The Journal of Immunology, vol. 176, no. 8, pp. 4940-4949, 2006.
P. Henneke, O. Takeuchi, R. Malley et al., “Cellular activation, phagocytosis, and bactericidal activity against group B streptococcus involve parallel myeloid differentiation factor 88-dependent and independent signaling pathways,” The Journal of Immunology, vol. 169, no. 7, pp. 3970-3977, 2002.
Y. Adachi, A. L. Kindzelskii, A. R. Petty et al., “IFN-γ primes RAW264 macrophages and human monocytes for enhanced oxidant production in response to CpG DNA via metabolic signaling: roles of TLR9 and myeloperoxidase trafficking,” The Journal of Immunology, vol. 176, no. 8, pp. 5033-5040, 2006.
H. Matsumoto, N. Hamada, A. Takahashi, Y. Kobayashi, and T. Ohnishi, “Vanguards of paradigm shift in radiation biology: radiation-induced adaptive and bystander responses,” Journal of Radiation Research, vol. 48, no. 2, pp. 97-106, 2007.
D. Tang, M. T. Lotze, H. J. Zeh, and R. Kang, “The redox protein HMGB1 regulates cell death and survival in cancer treatment,” Autophagy, vol. 6, no. 8, pp. 1181-1183, 2010.
H. Zhang. X. Gao, J. Zhao et al., “Differential gene expression profiles of DNA repair genes in esophageal cancer cells after X-ray irradiation,” Chinese Journal of Cancer, vol. 29, no. 10, pp. 865-872, 2010.
M. L. Brezniceanu, K. Volp, S. Bosser et al., “HMGB1 inhibits cell death in yeast and mammalian cells and is abundantly expressed in human breast carcinoma,” The FASEB Journal, vol. 17, no. 10, pp. 1295-1297, 2003.
T. E. Schmid and G. Multhoff, “Radiation-induced stress proteins—the role of heat shock proteins (HSP) in anti-tumor responses,” Current Medicinal Chemistry, vol. 19, no. 12, pp. 1765-1770, 2012.
Y. Suzuki, K. Mimura, Y. Yoshimoto et al., “Immunogenic tumor cell death induced by chemoradiotherapy in patients with esophageal squamous cell carcinoma,” Cancer Research, vol. 72, no. 16, pp. 3967-3976, 2012.
J. Tian, A. M. Avalos, S. Y. Mao et al., “Toll-like receptor 9-dependent activation by DNA-containing immune complexes is mediated by HMGB1 and RAGE,” Nature Immunology, vol. 8, no. 5, pp. 487-496, 2007.
S. Ivanov, A. Dragoi, X. Wang et al., “A novel role for HMGB1 in TLR9-mediated inflammatory responses to CpG-DNA,” Blood, vol. 110, no. 6, pp. 1970-1981, 2007.
M. E. Bianchi, “HMGB1 loves company,” Journal of Leukocyte Biology, vol. 86, no. 3, pp. 573-576, 2009.
H. S. Hreggvidsdottir, T. Ostberg, H. Wahamaa et al., “The alarmin HMGB1 acts in synergy with endogenous and exogenous danger signals to promote inflammation,” Journal of Leukocyte Biology; vol. 86, no. 3, pp. 655-662, 2009.
H. Yanai, T. Ban, Z. Wang et al., “HMGB proteins function as universal sentinels for nucleic-acid-mediated innate immune responses,” Nature, vol. 462, no. 7269, pp. 99-103, 2009.
Z. M. Bamboat, V. P. Balachandran, L. M. Ocuin, H. Obaid, G. Plitas, and R. P. DeMatteo, “Toll-like receptor 9 inhibition confers protection from liver ischemia-reperfusion injury,” Hepatology, vol. 51, no. 2, pp. 621-632, 2010.
C. Wang. G. Fei, Z. Liu et al., “HMGB1 was a pivotal synergistic effector for CpG oligonucleotide to enhance the progression of human lung cancer cells.” Cancer Biology & Therapy, vol. 13, no. 9, pp. 727-736, 2012.
E. A. Pasheva, I. G. Pashev, and A. Favre, “Preferential binding of high mobility group 1 protein to UV-damaged DNA: role of the COOH-terminal domain,” Journal of Biological Chemistry, vol. 273, no. 38, pp. 24730-24736, 1998.
H. Huang, J. Evankovich, W. Yan et al., “Endogenous histones function as alarmins in sterile inflammatory liver injury through Toll-like receptor 9 in mice,” Hepatology, vol. 54, no. 3, pp. 999-1008, 2011.
K. Yasuda, P. Yu, C. J. Kirschning et al., “Endosomal translocation of vertebrate DNA activates dendritic cells via TLR9-dependent and -independent pathways,” The Journal of Immunology, vol. 174, no. 10, pp. 6129-6136, 2005.
H. Wagner and S. Bauer, “All is not Toll: new pathways in DNA recognition,” The Journal of experimental medicine, vol. 203, no. 2, pp. 265-268, 2006.
V. Hornung and E. Latz, “Intracellular DNA recognition,” Nature Reviews Immunology, vol. 10, no. 2, pp. 123-130, 2010.
S. Wolff, “The adaptive response in radiobiology: evolving insights and implications,” Environmental Health Perspectives, vol. 106, supplement 1, pp. 277-283, 1998.
J. D. Shadley and S. Wolff, “Very low doses of X-rays can cause human lymphocytes to become less susceptible to ionizing radiation,” Mutagenesis, vol. 2, no. 2, pp. 95-96, 1987.
C. Mothersill, K. O.'Malley, and C. B. Seymour, “Characterisation of a bystander effect induced in human tissue explant cultures by low let radiation,” Radiation Protection Dosimetry, vol. 99, no. 1-4, pp. 163-167, 2002.
M. Buonanno, S. M. de Toledo, D. Pain, and E. I. Azzam, “Long term consequences of radiation-induced bystander effects depend on radiation quality and dose and correlate with oxidative stress,” Radiation Research, vol. 175, no. 4, pp. 405-415, 2011.
K. Suzuki, M. Ojima, S. Kodama, and M. Watanabe, “Radiation induced DNA damage and delayed induced genomic instability,” Oncogene, vol. 22, no. 45, pp. 6988-6993, 2003.
N. Hamada, H. Matsumoto, T. Hara, and Y. Kobayashi, “Intercellular and intracellular signaling pathways mediating ionizing radiation-induced bystander effects,” Journal of Radiation Research, vol. 48, no. 2, pp. 87-95, 2007.
S. Tapio and V. Jacob, “Radioadaptive response revisited,” Radiation and Environmental Biophysics, vol. 46, no. 1, pp. 1-12, 2007.
T. K. Hei, H. Zhou, V. N. Ivanov, M. Hong, H. B. Lieberman, D. J. Brenner, et al., Mechanism of radiation-induced bystander effects: a unifying model, J. Pharm. Pharmacol. 60 (2008) 943-950.
H. Matsumoto, M. Tomita, K. Otsuka, M. Hatashita, N. Hamada, Nitric oxide is a key molecule serving as a bridge between radiation-induced bystander and adaptive responses, Curr. Mol. Pharmacol. 4 (2011) 126-134.
D. Klokov, T. Criswell, K. S. Leskov, S. Araki, L. Mayo, D. A. Boothman, IR-inducible clusterin gene expression: a protein with potential roles in ionizing radiation-induced adaptive responses, genomic instability, and bystander effects, Mutat. Res. 568 (2004) 97-110.
N. Hamada, H. Matsumoto, T. Hara, Y. Kobayashi, Intercellular and intracellular signaling pathways mediating ionizing radiation-induced bystander effects, J. Radiat. Res. 48 (2007) 87-95.
J. M. Kaminski, E. Shinohara, J. B. Summers, K. J. Niermann, A. Morimoto, J. Brousal, The controversial abscopal effect, Cancer Treat, Rev. 31 (2005) 159-172.
C. Trainor, K. T. Butterworth, C. K. McGarry, S. J. McMahon, J. M. O'Sullivan, A. R. Hounsell. K. M. Prise, DNA damage responses following exposure to modulated radiation fields, PLoS One 7 (2012) e43326.
S. A. Lorimore, E. G. Wright, Radiation-induced genomic instability and bystander effects: related inflammatory-type responses to radiation induced stress and injury?, Int J. Radiat. Biol. 79 (2003) 15-25.
S. Desai, A. Kumar, S. Laskar. B. N. Pandey, Cytokine profile of conditioned medium from human tumor cell lines after acute and fractionated doses of gamma radiation and its effect on survival of bystander tumor cells, Cytokine 61 (2013) 54-62.
A. Facoetti, F. Pasi, R. Nano. Some considerations for the study of TGFbeta in medium of irradiated T98G cells: activation, release and consumption, Anticancer Res. 30 (2010) 3341-3344.
M. Natarajan, C. F. Gibbons, S. Mohan, S. Moore, M. A. Kadhim, Oxidative stress signalling: a potential mediator of tumour necrosis factor alpha-induced genomic instability in primary vascular endothelial cells, Br. J. Radiol. 80 (2007) S13-S22.
M. V. Sokolov, J. S. Dickey, W. M. Bonner, O. A. Sedelnikova, Gamma-H2AX in bystander cells: not just a radiation-triggered event, a cellular response to stress mediated by intercellular communication. Cell Cycle 6 (2007) 2210-2212.
J. S. Dickey, B. J. Baird, C. E. Redon, M. V. Sokolov, O. A. Sedelnikova, W. M. Bonner, Intercellular communication of cellular stress monitored by gamma-H2AX induction, Carcinogenesis 30 (2009) 1686-1695.
G. Nelson, J. Wordsworth, C. Wang, D. Jurk, C. Lawless, C. Martin-Ruiz, T. von Zglinicki, A senescent cell bystander effect: senescence-induced senescence, Aging Cell 11 (2012) 345-349.
J. P. Coppé, C. K. Patil, F. Rodier, Y. Sun, D. P. Muñoz, J. Goldstein, P. S. Nelson, et al., Senescence-associated secretory phenotypes reveal cell nonautonomous functions of oncogenic RAS and the p53 tumor suppressor, PLoS Biol. 6 (2008) 2853-2868.
J. P. Coppé, P. Y. Desprez, A. Krtolica, J. Campisi, The senescence-associated secretory phenotype: the dark side of tumor suppression, Ann. Rev. Pathol. 5 (2010) 99-118.
J. C. Acosta, A. O'Loghlen, A. Banito, M. V. Guijarro, A. Augert, S. Raguz, M. Fumagalli, et al., Chemokine signaling via the CXCR2 receptor reinforces senescence, Cell 133 (2008) 1006-1018.
C. E. Redon, J. S. Dickey, A. J. Nakamura, I. G. Kareva, D. Naf, S. Nowsheen, et al., Tumors induce complex DNA damage in distant proliferative tissues in vivo. Proc. Natl. Acad. Sci. USA 107 (2010) 17992-17997.
A. V. Ermakov, M. S. Konkova, S. V. Kostiuk, N. N. Veiko, DNA-signaling pathway mediating development of a radiation-induced bystander effect in human cells, Radiat. Biol. Radioecol. 51 (2011) 651-659.
S. V. Kostyuk, A. V. Ermakov, A. Y. Alekseeva, T. D. Smirnova, K. V. Glebova, L. V. Efremova, et al., Role of extracellular DNA oxidative modification in radiation induced bystander effects in human endotheliocytes, Mutat. Res. 729 (2012) 52-60.
A. V. Ermakov, M. S. Konkova, S. V. Kostyuk, T. D. Smirnova. E. M. Malinovskaya, L. V. Efremova, N. N. Veiko, An extracellular DNA mediated bystander effect produced from low dose irradiated endothelial cells, Mutat Res. 712 (2011) 1-10.
A. V. Ermakov, M. S. Konkova, S. V. Kostyuk, N. A. Egolina, L. V. Efremova, N. N. Veiko, Oxidative stress as a significant factor for development of an adaptive response in irradiated and nonirradiated human lymphocytes after inducing the bystander effect by low-dose X-radiation, Mutat. Res. 669 (2009) 155-161.
A. V. Ermakov, M. S. Konkova, S. V. Kostiuk, E. A. Kalashnikova, S. N. Kokarovtseva, N. A. Egolina, N. N. Veiko, CpG-DNA inhibits cell reactions accompanied with the development of the adaptive response in human lymphocytes after low-dose X-ray exposure, Radiat. Biol. Radioecol. 49 (2009) 34-41.
A. V. Ermakov, M. S. Konkova, S. V. Kostiuk, E. S. Ershova, N. A. Egolina, N. N. Veiko, Extracellular DNA fragments from culture medium of low-dose irradiated human lymphocyte trigger instigating of the oxidative stress and the adaptive response in non-irradiated bystander lymphocytes, Radiat. Biol. Radioecol. 48 (2008) 553-564.
A. V. Ermakov, S. V. Kostyuk, M. S. Konkova, N. A. Egolina, E. M. Malinovskaya, N. N. Veiko, Extracellular DNA fragments, Ann. N.Y. Acad. Sci. 1137 (2008) 41-46.
A. V. Ermakov, S. V. Kostiuk, N. A. Egolina, E. A. Kalashnikova, S. N. Kokarovtseva, E. M. Malinovskaia, N. N. Veiko, Stress signaling between human lymphocytes after induction of bystander effect by exposure to ionizing radiation in adaptive doses, Radiat. Biol. Radioecol. 47 (2007) 650-657.
P. B. Gahan, M. Stroun, The virtosome—a novel cytosolic informative entity and intercellular messenger, Cell Biochem. Funct. 28 (2010) 529-538.
N. Hamada, G. Schettino, G. Kashino, M. Vaid. K. Suzuki, S. Kodama, et al., Histone H2AX phosphorylation in normal human cells irradiated with focused ultrasoft X rays: evidence for chromatin movement during repair, Radiat. Res. 166 (2006) 31-38.
M. Gandhi, V. N. Evdokimova, K. T. Cuenco, M. N. Nikiforova, L. M. Kelly, J. R. Stringer, et al., Homologous chromosomes make contact at the sites of double strand breaks in genes in somatic G0/G1-phase human cells, Proc. Natl. Acad. Sci. USA 109 (2012) 9454-9459.
M. K. Vartiaineu, Nuclear actin dynamics—from form to function, FEBS Lett. 582 (2008) 2033-2040.
C. W. Gourlay, K. R. Ayscough. The actin cytoskeleton: a key regulator of apoptosis and ageing?, Nat Rev. Mol. Cell Biol. 6 (2005) 583-589.
J. Sroka, Z. Madeja, Reactive oxygen species in regulation of cell migration. The role of thioredoxin reductase, Postepy Biochem. 55 (2009) 145-152.
G. N. Barber, Cytoplasmic DNA innate immune pathways, Immunol. Rev. 243 (2011) 99-108.
Y. Adachi, A. L. Kindzelskii, A. R. Petty, J. B. Huang, N. Maeda, S. Yotsumoto, et al., IFN-gamma primes RAW264 macrophages and human monocytes for enhanced oxidant production in response to CpG DNA via metabolic signaling: roles of TLR9 and myeloperoxidase trafficking. J. Immunol. 176 (2006) 5033-5040.
P. B. Gahan, M. Stroun, The biology of circulating nucleic acids in plasma and serum. In: Y. Yo Kikuchi, E. Y. Rykova (Eds) Extracellular nucleic acids. In: NAMB Series Nucleic Acids and Molecular Biology. Springer, Berlin, 2010.
I. Mittra, N. K. Nair, P. K. Mishra, Nucleic acids in circulation: are they harmful to the host?, J Biosci. 37 (2012) 301-312.
S. V. Kostiuk, I. A. Zamulaeva, R. K. Agapova, A. V. Ermakov, A. S. Saenko, N. V. Orlova, et al., The changing of cell-free DNA properties of peripheral blood and TCR-mutant cell frequency in individuals exposed to ionizing radiation, Radiat. Biol. Radioecol. 48 (2008) 5-13.
N N. Veiko, N. O. Shubaeva, S. M. Ivanova, A. I. Speranskii, N. A. Lyapunova, D. M. Spitkovskii, Blood serum DNA in patients with rheumatoid arthritis is considerably enriched with fragments of ribosomal repeats containing immunostimulatory CpG-motifs, Bull. Exp. Biol. Med. 142 (2006) 313-316.
N. N. Veiko, N. V. Bulycheva, O. A. Roginko, R. V. Veiko, E. S. Ershova, O. A. Kozdoba, et al., Ribosomal repeat in cell free DNA as a marker for cell death, Biomed. Chem. 2 (2008) 198-207.
L. Li, Y. J. Choi, K. M. Lee, H. Sung, S. K. Park S K, I. Oze, et al., DNA methylation in peripheral blood: a potential biomarker for cancer molecular epidemiology, J. Epidemiol. 22 (2012): 384-394.
E. Y. Rykova, E. S. Morozkin, A. A. Ponomaryova, E. M. Loseva, I. A. Zaporozhchenko, N. V. Cherdyntseva, et al., Cell-free and cell-bound circulating nucleic acid complexes: mechanisms of generation, concentration and content, Expert Opin. Biol. Ther. 12 (2012) S141-153.
S. Perrier, J. Hau, D. Gasparutto, J. Cadet, A. Favier J-L, Ravanat, characterization of lysine-guanine cross-links upon one-electron oxidation of a guanine-containing oligonucleotide in the presence of a trilysine peptide, J. Am. Chem. Soc. 128 (2006) 5703-5710.
X. Xu, J. G. Muller, Y. Ye, C. J. Burrows, DNA-protein crosslinks between guanine and lysine depend on the mechanism of oxidation for formation of C5 Vs C8 guanosine adducts, J. Am. Chem. Soc. 130 (2008) 703-709.
N. R. Jena. DNA damage by reactive species: mechanisms, mutation and repair, J. Biosci. 37 (2012) 503-517.
H. Bartsch, J. Nair, Oxidative stress and lipid peroxidation-derived DNA lesions in inflammation driven carcinogenesis, Cancer Detect. Prev. 28 (2004) 385-391.
O. A. Sedelnikova, C. E. Redon, J. S. Dickey, A. J. Nakamura, A. G. Georgakilas, W. M. Bonner, Role of oxidatively induced DNA lesions in human pathogenesis, Mutat. Res. 704 (2010) 152-159.
S. Nowsheen, R. L. Wukovich, K. Aziz, P. T. Kalogerinis, C. C. Richardson, M. I. Panayiotidis, et al., Accumulation of oxidatively induced clustered DNA lesions in human tumor tissues, Mutat. Res. 674 (2009) 131-136.
J. Cadet, J. L. Ravanat, M. TavernaPorro, H. Menoni, D. Angelov. Oxidatively generated complex DNA damage: tandem and clustered lesions, Cancer Lett. 31 (2012) 5-15.
N. R. Jena, P. C. Mishra, Formation of ring-opened and rearranged products of guanine: mechanisms and biological significance, Free Radical. Biol. Med. 53\(2012) 81-94.
A. Valavanidis, T. Vlachogianni, C. Fiotakis, 8-hydroxy-20-deoxyguanosine (8-OHdG): a critical biomarker of oxidative stress and carcinogenesis, J. Environ. Sci. Health C Environ. Carcinog. Ecotoxicol. Rev. 27 (2009) 120-139.
A. R. Collins, J. Cadet, L. Möller, H. E. Poulsen, J. Viña, Are we sure we know how to measure 8-oxo-7,8-dihydroguanine in DNA from human cells?, Arch Biochem. Biophys. 423 (2004) 57-65.
J. Cadet, C. D'Ham, T. Douki, J. P. Pouget, J. L. Ravanat, S. Sauvaigo, Facts and artifacts in the measurement of oxidative base damage to DNA, Free Radical. Res. 29 (1998) 541-550.
http://fabad.org/fabad.org/pdf/volum30/issue2/100-113.pdf.
J. L. Ravanat, P. Di Mascio, G. R. Martinez, M. H. Medeiros, J. Cadet, Singlet oxygen induces oxidation of cellular DNA, J. Biol. Chem. 275 (2000) 40601-40604.
K. Ishikawa, K. Takenaga, M. Akimoto, N. Koshikawa, A. Yamaguchi, H. Imanishi, et al., ROS-generating mitochondrial DNA mutations can regulate tumor cell metastasis, Science 320 (2008) 661-664.
T. P. Szatrowski, C. F. Nathan, Production of large amounts of hydrogen peroxide by human tumor cells, Cancer Res. 51 (1991) 794-798.
S. Kawanishi, Y. Hiraku, S. Pinlaor, N. Ma, Oxidative and nitrative DNA damage in animals and patients with inflammatory diseases in relation to inflammation-related carcinogenesis, Biol. Chem. 387 (2006) 365-372.
S. L. Payne, B. Fogelgren, A. R. Hess, E. A. Seftor, E. L. Wiley, S. F. Fong, K. Csiszar, et al., Lysyl oxidase regulates breast cancer cell migration and adhesion through a hydrogen peroxide-mediated mechanism, Cancer Res. 65 (2005) 11429-11436.
M. Geiszt, T. L. Leto, The Nox family of NAD(P)H oxidases: host defense and beyond, J. Biol. Chem. 279 (2004) 51715-51718.
J. E. Klaunig, Z. Wang, X. Pu, S. Zhou, Oxidative stress and oxidative damage in chemical carcinogenesis, Toxicol. Appl. Pharmacol. 254 (2011) 86-99.
J. A. Petros, A. K. Baumann, E. Ruiz-Pesini, M. B. Amin, C. Q. Sun, J. Hall, et al., MtDNA mutations increase tumorigenicity in prostate cancer. Proc. Natl. Acad. Sci. USA 102 (2005) 719-724.
J. L. Arbiser, J. Petros, R. Klafter, B. Govindajaran, E. R. McLaughlin, L. F. Brown, et al., Reactive oxygen generated by Nox1 triggers the angiogenic switch, Proc. Natl. Acad. Sci. USA 99 (2002) 715-720.
S. D. Lim, C. Sun, J. D. Lambeth, F. Marshall, M. Amin, L. Chung, et al., Increased Nox1 and hydrogen peroxide in prostate cancer, Prostate 62 (2005) 200-207.
T. D. Oberley, Oxidative damage and cancer, Am. J. Pathol. 160 (2002) 403-408.
A. S. Kamiguti, L. Serrander, K. Lin, R. J. Harris, J. C. Cawley, D. J. Allsup, J. R. Slupsky, et al., Expression and activity of NOX5 in the circulating malignant B cells of hairy cell leukemia, J. Immunol. 175 (2005) 8424-8430.
A. Petry, M. Weitnauer, A. Görlach, Receptor activation of NADPH oxidases, Antioxid. Redox Signal. 13 (2010) 467-487.
A. M. Knaapen, N. Gilngor, R. P. Schins, P. J. Borm, F. J. Van Schooten, Neutrophils and respiratory tract DNA damage and mutagenesis: a review, Mutagenesis 21(2006) 225-236.
O. Handa, Y. Naito, T. Yoshikawa, Helicobacter pylori: a ROS-inducing bacterial species in the stomach, Inflamm. Res. 59 (2010) 997-1003.
K. A. Graham, M. Kulawiec, K. M. Owens, X. Li, M. M. Desouki, D. Chandra, K. K. Singh, NADPH oxidase 4 is an oncoprotein localized to mitochondria, Cancer Biol. Ther. 10 (2010) 223-231.
C. Guichard, E. Pedruzzi, M. Fay, S. Ben Mkaddem, N. Coant, F. Daniel, E. Ogier-Denis, The Nox/Duox family of ROS-generating NADPH oxidases, Med. Sci. (Paris) 22 (2006) 953-959.
J. Liu. W. Qu, M. B. Kadiiska, Role of oxidative stress in cadmium toxicity and carcinogenesis, Toxicol. Appl. Pharmacol. 238 (2009) 209-214.
J. E. Klaunig, Y. Xu, S. Bachowski, J. Jiang, Free-radical oxygen-induced changes in chemical carcinogenesis, in: K. B. Wallace (Ed.), Free Radical Toxicology, Taylor & Francis, London, 1997, pp. 375-400.
W. F. Heidenreich, E. G. Luebeck, W. D. Hazelton, H. G. Paretzke, S. H. Moolgavkar, Multistage models and the incidence of cancer in the cohort of atomic bomb survivors, Radiat. Res. 158 (2002) 607-614.
Y. J. Huang, B. B. Zhang, N. Ma, M. Murata, A. Z. Tang, G. W. Huang, Nitrative and oxidative DNA damage as potential survival biomarkers for nasopharyngeal carcinoma, Med. Oncol. 28 (2011) 377-384.
S. Toyokuni. K. Okamoto, J. Yodoi, H. Hiai, Persistent oxidative stress in cancer, FEBS Lett. 358 (1995) 1-3.
G. Romano, A. Sgambato, R. Mancini, G. Capelli, M. R. Giovagnoli, G. Flamini, et al., 8-hydroxy-20-deoxyguanosine in cervical cells: correlation with grade of dysplasia and human papillomavirus infection, Carcinogenesis 21 (2000) 1143-1147.
J. V. Räsänen, E. I. Sihvo, M. O. Ahotupa, M. A. Farkkila, J. A. Salo, The expression of 8-hydroxydeoxyguanosine in oesophageal tissues and tumours, Eur. J. Surg. Oncol. 33 (2007) 1164-1168.
M. Nagashima, H. Tsuda, S. Takenoshita, Y. Nagamachi, S. Hirohashi, J. Yokota, H. Kasai, 8-hydroxydeoxyguanosine levels in DNA of human breast cancer are not significantly different from those of non-cancerous breast tissues by the HPLC-ECD method, Cancer Lett. 90 (1995) 157-162.
P. Jaloszynski, P. Jaruga, R. Olinski, W. Biczysko, W. Szyfter, E. Nagy, et al., Oxidative DNA base modifications and polycyclic aromatic hydrocarbon DNA adducts in squamous cell carcinoma of larynx, Free Radical. Res. 37 (2003) 231-240.
J. Fang, T. Seki, H. Maeda, Therapeutic strategies by modulating oxygen stress in cancer and inflammation, Adv. Drug Deliv. Rev. 61 (2009) 290-302.
S. C. Lim, J. E. Choi, H. S. Kang, S. I. Han, Ursodeoxycholic acid switches oxaliplatin-induced necrosis to apoptosis by inhibiting reactive oxygen species production and activating p53-caspase 8 pathway in HepG2 hepatocellular carcinoma, Int. J. Cancer 126 (2010) 1582-1595.
S. Afzal, S. A. Jensen, J. B. Sorensen, T. Henriksen, A. Weimann, H. E. Poulsen, Oxidative damage to guanine nucleosides following combination chemotherapy with 5-fluorouracil and oxaliplatin, Cancer Chemother. Pharmacol. 69 (2012) 301-307.
P. Atukeren, B. Yavuz, H. O. Soydinc, S. Purisa, H. Camlica, M. K. Gumustas, I. Balcioglu, Variations in systemic biomarkers of oxidative/nitrosative stress and DNA damage before and during the consequent two cycles of chemotherapy in breast cancer patients, Clin. Chem. Lab. Med. 48 (2010) 1487-1495.
M. Mousseau, H. Faure, I. Hininger, M. Bayet-Robert, A. Favier, Leukocyte 8-oxo-7,8-dihydro-20-deoxyguanosine and comet assay in epirubicin-treated patients, Free Radical. Res. 39 (2005) 837-843.
T. Kaneko, S. Tahara, T. Taguchi, H. Kondo, Accumulation of oxidative DNA damage, 8-oxo-20-deoxyguanosine, and change of repair systems during in vitro cellular aging of cultured human skin fibroblasts, Mutat. Res. 487 (2001) 19-30.
M. C. Gomez-Cabrera, F. Sanchis-Gomar, R. Garcia-Valles, H. Pareja-Galeano, J. Gambini, C. Borras, J. Viña, Mitochondria as sources and targets of damage in cellular aging, Clin. Chem. Lab. Med. 50 (2012) 1287-1295.
E. Doria, D. Buonocore, A. Focarelli, F. Marzatico, Relationship between human aging muscle and oxidative system pathway, Oxid. Med. Cell Longev. (2012) 830257.
A. Siomek, D. Gackowski, R. Rozalski, T. Dziaman, A. Szpila, J. Guz, R. Olinski, Higher leukocyte 8-oxo-7,8-dihydro-20-deoxyguanosine and lower plasma ascorbate in aging humans?, Antioxid Redox Signal. 9 (2007) 143-150.
S. K. Pazhanisamy, H. Li, Y. Wang, I. Batinic-Haberle, D. Zhou, NADPH oxidase inhibition attenuates total body irradiation-induced haematopoietic genomic instability, Mutagenesis 26 (2011) 431-435.
S. Tahara. T. Kaneko, Susceptibility of mouse splenic cells to oxidative DNA damage by X-ray irradiation, Biol. Pharm. Bull. 27 (2004) 105-108.
W. Sudprasert, P. Navasumrit, M. Ruchirawat, Effects of low-dose gamma radiation on DNA damage, chromosomal aberration and expression of repair genes in human blood cells, Int. J. Hyg. Environ. Health 209 (2006) 503-511.
K. Roszkowski, D. Gackowski, R. Rozalski, T. Dziaman, A. Siomek, J. Guz, et al., Small field radiotherapy of head and neck cancer patients is responsible for oxidatively damaged DNA/oxidative stress on the level of a whole organism. Int. J. Cancer 123 (2008) 1964-1967.
T. Bergström, C. Ersson, J. Bergman, L. Möller, Vitamins at physiological levels cause oxidation to the DNA nucleoside deoxyguanosine and to DNA—alone or in synergism with metals, Mutagenesis 27 (2012) 511-517.
H. Schwarzenbach, D. S. Hoon, K. Pantel, Cell-free nucleic acids as biomarkers in cancer patients, Nat. Rev. Cancer 11 (2011) 426-437.
K. Kuroi, C. Tanaka, M. Toi, Plasma nucleosome levels in node-negative breast cancer patients, Breast Cancer 6 (1999) 361-364.
N. Umetani, J. Kim, S. Hiramatsu, H. A. Reber, O. J. Hines, A. J. Bilchik, D. S. Hoon. Increased integrity of free circulating DNA in sera of patients with colorectal or periampullary cancer: direct quantitative PCR for ALU repeats. Clin. Chem. 52 (2006) 1062-1069.
S. Mueller, S. Holdenrieder, P. Stieber, T. Haferlach, A. Schalhorn, J. Braess, et al., Early prediction of therapy response in patients with acute myeloid leukemia by nucleosomal DNA fragments, BMC Cancer 6 (2006) 143.
I. V. Gannushkina, M. L. Farago, A. L. Antelava, M. V. Baranchikova, N. N. Veiko, Hemodynamic effect of plasma DNA, Vestnik RAMN 5 (1998) 16-22.
M. B. Giacona, G. C. Ruben, K. A. Iczkowski, T. B. Roos, D. M. Porter, G. D. Sorenson, Cell-free DNA in human blood plasma: length measurements in patients with pancreatic cancer and healthy controls, Pancreas 17 (1998) 89-97.
T. L. Wu, D. Zhang, J. H. Chia, K. H. Tsao, C. F. Sun, J. T. Wu, Cell-free DNA: measurement in various carcinomas and establishment of normal reference range, Clin. Chim. Acta 321 (2002) 77-87.
S. Jahr, H. Hentze, S. Englisch, D. Hardt, F. O. Fackelmayer, R. D. Hesch, R. Knippers, DNA fragments in the blood plasma of cancer patients: quantitations and evidence for their origin from apoptotic and necrotic cells, Cancer Res. 61 (2001) 1659-1665.
B. Schmidt, S. Weickmann, C. Witt, M. Fleischhacker, Integrity of cell-free plasma DNA in patients with lung cancer and nonmalignant lung disease, Ann. N.Y. Acad. Sci. 1137 (2008) 207-213.
B. G. Wang, H. Y. Huang, Y. C. Chen, R. E. Bristow, K. Kassauei, C. C. Cheng, et al., Increased plasma DNA integrity in cancer patients, Cancer Res. 63 (2003) 3966-3968.
U. Deligezer, Y. Eralp, E. Z. Akisik, E. E. Akisik, P. Saip, E. Topuz, N. Dalay, Effect of adjuvant chemotherapy on integrity of free serum DNA in patients with breast cancer, Ann. N.Y. Acad. Sci. 1137 (2008) 175-179.
W. W. Jiang, M. Zahurak, D. Goldenberg, Y. Milman, H. L. Park, W. H. Westra, et al., Increased plasma DNA integrity index in head and neck cancer patients, Int. J. Cancer 119 (2006) 2673-2676.
S. Holdenrieder, A. Burges, O. Reich, F. W. Spelsberg, P. Stieber, DNA integrity in plasma and serum of patients with malignant and benign diseases, Ann. N. Y. Acad. Sci. 1137 (2008) 162-170.
D. S. Pisetsky, DNA as a marker of cell death in systemic lupus erythematosus, Rheum. Dis. Clin. North Am. 30 (2004) 575-587.
K. Smallbone, R. A. Gatenby, R. J. Gillies, P. K. Maini, D. J. Gavaghan, Metabolic changes during carcinogenesis: potential impact on invasiveness, J. Theor. Biol. 244 (2007) 703-713.
M. Pylväs, U. Puistola. L. Laatio, S. Kauppila, P. Karihtala, Elevated serum 8-OHdG is associated with poor prognosis in epithelial ovarian cancer, Anticancer Res. 31 (2011) 1411-1415.
T. Sato, H. Takeda, S. Otake, J. Yokozawa, S. Nishise, S. Fujishima, et al., Increased plasma levels of 8-hydroxydeoxyguanosine are associated with development of colorectal tumors, J. Clin. Biochem. Nutr. 47 (2010) 59-63.
P. Loseva, S. Kostyuk, E. Malinovskaya, N. Clement, C. A. Dechesne, C. Dani, et al., Extracellular DNA oxidation stimulates activation of NRF2 and reduces the production of ROS in human mesenchymal stem cells, Expert Opin. Biol. Ther. 12 (2012) S85-97.
I. N. Vasilyeva, Low-molecular-weight DNA in blood plasma as an index of the influence of ionizing radiation, Ann. N.Y. Acad. Sci. 945 (2001) 221-228.
E. V. Evdokimovski, N. E. Gubina, T. E. Ushakova, A. I. Gaziev, Changes of mitochondrial DNA/nuclear DNA ratio in the blood serum following X-ray irradiation of mice at various doses, Radiat. Biol. Radioecol. 52 (2012) 565-571.
C. Cheng, M. Omura-Minamisawa, Y. Kang, T. Hara, I. Koike, T. Inoue, Quantification of circulating cell-free DNA in the plasma of cancer patients during radiation therapy, Cancer Sci. 100 (2009) 303-309.
J. Jylhävä, M. Jylhä, T. Lehtimäki, A. Hervonen, M. Hurme, Circulating cell-free DNA is associated with mortality and inflammatory markers in nonagenarians: the vitality 90+ study, Exp. Gerontol. 47 (2012) 372-378.
J. Jylhävä, T. Nevalainen, S. Marttila, M. Jylhä, A. Hervonen, M. Hurme, Characterization of the role of distinct plasma cell-free DNA species in age associated inflammation and frailty, Aging Cell 12 (2013) 388-397.
H. Wagner, S. Bauer, All is not Toll: new pathways in DNA recognition, J. Exp. Med. 203 (2006) 265-268.
V. Hornung, E. Latz, Intracellular DNA recognition, Nat. Rev. Immunol. 10 (2010) 123-130.
C. Wang, G. Fei, Z. Liu, Q. Li, Z. Xu, T. Ren, HMGB1 was a pivotal synergistic effector for CpG oligonucleotide to enhance the progression of human lung cancer cells, Cancer Biol. Ther. 13 (2012) 727-736.
H. Yoshida, M. Nishikawa, T. Kiyota, H. Toyota, Y. Takakura, Increase in CpG DNA-induced inflammatory responses by DNA oxidation in macrophages and mice, Free Radical. Biol. Med. 51 (2011) 424-431.
E. A. Pasheva, I. G. Pashev, A. Favre, Preferential binding of high mobility group 1 protein to UV-damaged DNA: role of the COOH-terminal domain, J. Biol. Chem. 273 (1998) 24730-24736.
S. V. Kostyuk, M. S. Konkova, E. S. Ershova, A. J. Alekseeva, T. D. Smirnova, S. V. Stualov, et al., An exposure to the oxidized DNA enhances both instability of genome and survival in cancer cells, PLOS One. Volume 8 Issue 10 ee77469 (October 2013).
L. V. Efremova, A. Y. Alekseeva, M. S. Konkova, S. V. Kostyuk, E. S. Ershova, T. D. Smirnova, et al., Extracellular DNA affects NO content in human endothelial cells, Bull. Exp. Biol. Med. 149 (2010) 196-200.
S. V. Kostyuk, V. J. Tabakov, V. V. Chestkov, M. S. Konkova, K. V. Glebova, G. V. Baydakova, et al., Oxidized DNA induces an adaptive response in human fibroblasts, Mutat. Res. 747-748 (2013) 6-8.
D. H. Adams, N. Diaz, P. B. Gahan, in vitro stimulation by tumour cell media of [3H]-thymidine incorporation by mouse spleen lymphocytes, Cell Biochem. Funct. 15 (1997) 119-126.
S. Nair, S. T. Doh, J. Y. Chan, A. N. Kong, L. Cai, Regulatory potential for concerted modulation of Nrf2- and Nfkb1-mediated gene expression in inflammation and carcinogenesis, Br. J. Cancer 99 (2008) 2070-2082.
G. Y. Chen, G. Nuñez, Sterile inflammation: sensing and reacting to damage, Nat. Rev. Immunol. 10 (2010) 826-837.
K. Suzuki, A. Mori, K. J. Ishii, J. Saito, D. S. Singer, D. M. Klinman, et al., Activation of target-tissue immune-recognition molecules by double-stranded polynucleotides, Proc. Natl. Acad. Sci. USA 96 (1999) 2285-2290.
L. Rock, J. J. Lai, H. Kono, Innate and adaptive immune responses to cell death, Immunol. Rev. 243 (2011) 191-205.
J. J. Choi, C. F. Reich 3rd, D. S. Pisetsky, The role of macrophages in the in vitro generation of extracellular DNA from apoptotic and necrotic cells, Immunology 115 (2005) 55-62.
P. Decker, H. Wolburg, H. G. Rammensee, Nucleosomes induce lymphocyte necrosis, Eur. J. Immunol. 33 (2003) 1978-1987.
D. C. García-Olmo, D. García-Olmo, Biological role of cell-free nucleic acids in cancer: the theory of genometastasis, Crit. Rev. Oncog. 18 (2013) 153-161.
A. Bergsmedh, J. Ehnfors, K. Kawane, N. Motoyama, S. Nagata, L. Holmgren, DNase II and the Chk2 DNA damage pathway form a genetic barrier blocking replication of horizontally transferred DNA, Mol. Cancer Res. 4 (2006) 187-195.
G. Serrano-Heras, C. Domínguez-Berzosa, E. Collantes, H. Guadalajara, D. García-Olmo, D. C. García-Olmo, NIH-3T3 fibroblasts cultured with plasma from colorectal cancer patients generate poorly differentiated carcinomas in mice, Cancer Lett. 316 (2012) 85-90.
E. Speina, K. D. Arczewska, D. Gackowski, M. Zielinska, A. Siomek, J. Kowalewski, et al., Contribution of hMTH1 to the maintenance of 8-oxoguanine levels in lung DNA of non-small-cell lung cancer patients, J. Natl. Cancer Inst. 97 (2005) 384-395.
M. R. Oliva, F. Ripoll, P. Muñiz, P. A. Iradi, R. Trullenque, V. Valls, et al., Genetic alterations and oxidative metabolism in sporadic colorectal tumors from a Spanish community. Mol. Carcinog. 18 (1997) 232-243.
P. Jaruga, T. H. Zastawny, J. Skokowski, M. Dizdaroglu, R. Olinski, Oxidative DNA base damage and antioxidant enzyme activities in human lung cancer, FEBS Lett. 341 (1994) 59-64.
S. Kondo, S. Toyokuni, Y. Iwasa, T. Tanaka, H. Onodera, H. Hiai, M. Imamura, Persistent oxidative stress in human colorectal carcinoma, but not in adenoma, Free Radic. Biol. Med. 27 (1999) 401-410.
D. C. Malins, R. Haimanot, Major alterations in the nucleotide structure of DNA in cancer of the female breast, Cancer Res. 51 (1991) 5430-5432.
A. Matsui, T. Ikeda, K. Enomoto, K. Hosoda, H. Nakashima, K. Omae, et al., Increased formation of oxidative DNA damage, 8-hydroxy-2′-deoxyguanosine, in human breast cancer tissue and its relationship to GSTP1 and COMT genotypes, Cancer Lett. 151 (2000) 87-95.
A. Kumar, M. C. Pant, H. S. Singh, S. Khandelwal, Role of OGG1 Ser326Cys polymorphism and 8-oxoguanine DNA damage in risk assessment of squamous cell carcinoma of head and neck in North Indian population, Mutat. Res. 726 (2011) 227-233.
K. Glebova et al., Oxidized extracellular DNA as a stress signal that may modify response to anticancer therapy, Cancer Lett. (2013), http://dx.doi.org/10.1016/j.canlet.2013.09.005
D. Gackowski, Z. Banaszkiewicz, R. Rozalski, A. Jawien, R. Olinski, Persistent oxidative stress in colorectal carcinoma patients, Int. J. Cancer. 101 (2002) 395-397.
M. Nagashima, H. Tsuda, S. Takenoshita, Y. Nagamachi, S. Hirohashi, J. Yokota, H. Kasai, 8-hydroxydeoxyguanosine levels in DNA of human breast cancer are not significantly different from those of non-cancerous breast tissues the HPLC-ECD method, Cancer Lett. 90 (1995) 157-162.

1. A method for diagnosing the oxidative damage encountered by a subject over a recent time period, comprising the steps of:
(a) obtaining a sample of blood or other biological fluid from said subject;
(b) removing all cells from the sample;
(c) extracting extracellular nucleic acid from the sample;
(d) measuring the percentage of oxidized nucleotides within the extracted extracellular nucleic acid or quantifying the total amount of oxidized nucleotides within the extracellular nucleic acid; and
(e) diagnosing the degree of oxidative damage that said subject encountered across the recent time period proportionate to the increase in the percentage of oxidized nucleotides above baseline levels, wherein baseline levels of oxidized nucleotides are calculated from either the same subject or as a per average amount of oxidized nucleotides obtained from a same-species population of said subject.

2. (canceled)

3. A method for monitoring oxidative damage in a subject who is afflicted by a chronic disease, comprising the steps of:
(a) obtaining a sample of blood or other biological fluid from said subject;
(b) removing all cells from the sample;
(c) extracting extracellular nucleic acid from the sample;
(d) measuring the percentage of oxidized nucleotides within the extracted extracellular nucleic acid or quantifying the total amount of oxidized nucleotides within the extracellular nucleic acid; and
(e) diagnosing the degree of oxidative damage that said subject accumulated over time proportionate to the increase in the percentage of oxidized nucleotides above baseline levels, wherein baseline levels of oxidized nucleotides are calculated from the same subject from an earlier period of time.

4-8. (canceled)

9. The method of claim 1, wherein the subject is selected from the group consisting of: human, mouse, rat, rabbit, guinea pig, dog, cat, pig, and monkey.

10-11. (canceled)

12. The method of claim 1, wherein the subject is profiled longitudinally and wherein the percentage of oxidized nucleotides is used for long-term monitoring of the effects of various environmental impacts.

13. The method of claim 12, wherein the environmental impact is environmental stress.

14. The method of claim 13, wherein the environmental stress is oxidative stress.

15. The method of claim 1, wherein said subject is profiled longitudinally and wherein the percentage of oxidized nucleotides is used for long-term or short-term monitoring of the effects of cancer therapy aimed to induce tumor cell death by increasing oxidative damage in cancer cells.

16. The method of claim 1, wherein the percentage of oxidized nucleotides is measured chemically or electrochemically.

17. The method of claim 1, wherein the percentage of oxidized nucleotides is measured using antibodies, aptamers, or fragments thereof.

18. The method of claim 1, wherein the percentage of oxidized nucleotides is measured enzymatically.

19. A method for evaluating the oxidative damage in a cell culture that was exposed to environmental stress, comprising the steps of:
(a) removing all cells from the cell culture sample;
(b) collecting the cell-free media from the cell culture sample;
(c) extracting extracellular nucleic acid from the cell culture sample;
(d) measuring the percentage of oxidized nucleotides within the extracted extracellular nucleic acid or quantifying the total amount of oxidized nucleotides within the extracellular nucleic acid; and
(e) determining the degree of oxidative damage that said cell culture experienced as a result of exposure to said environmental stress proportionate to the increase in the percentage of oxidized nucleotides above baseline levels, wherein baseline levels of oxidized nucleotides are calculated from a similarly cultured cell line.

20. The method of claim 19, wherein the cell culture comprised primary cells explanted from an organism.

21. The method of claim 20, wherein said environmental stress is a treatment with a compound with cell phenotype or gene expressing altering abilities.

22. The method of claim 20, wherein said environmental stress is a damaging stress.

23. A method for abating the side effects of chemotherapy in a human cancer patient, comprising removing extracellular nucleic acid from said patient's blood.

24. (canceled)

25. The method of claim 23, wherein said extracellular nucleic acid is removed by hemosorbtion or plasmapheresis with a DNA-binding sorbent.

26. (canceled)

27. The method of claim 25, wherein said DNA-binding sorbent is silica.

28. The method of claim 1, wherein the extracellular nucleic acid is extracellular DNA.

29. The method of claim 1, wherein the oxidized nucleotide is 8-hydroxy-2′-deoxyguanosine.

30. A method of conditioning stem cells to make said cells more resistant to environmental stress comprising the steps of:
(a) expanding said cells in a cell culture medium; and
(b) adding an artificially created preparation of oxidized genomic DNA to said cells.

31. A method of treating oxidative damage in a subject comprising administering to said subject a composition comprising an agent that binds oxidized extracellular nucleic acid.

32. (canceled)

33. The method of claim 31, wherein the agent binds one or more of modified nucleobases selected from the group consisting of: 8-hydroxyadenine, 8-hydroxy-2′-deoxyguanosine, thymine glycol, Fapy-guanine, 5-hydroxymethyl-2′-deoxyuridine, and Fapy-adenine.

34. The method of claim 33, wherein the agent is an antibody or a fragment thereof.

35. The method of claim 31, wherein the disease or condition is selected from the group consisting of: cancer, Leber's hereditary optic neuropathy, Parkinson's disease, multiple sclerosis, Alzheimer's disease, schizophrenia, chronic renal failure, Fanconi anaemia, type 1 diabetes, type II diabetes, coronary artery disease, myocardial infarction, hypertension, atherosclerosis, rheumatoid arthritis, and disease characterized by mitochondrial dysfunction.

36. The method of claim 35, wherein the cancer is selected from the group consisting of: breast cancer, prostate cancer, epithelial ovarian cancer, and lung cancer.

37. The method of claim 31, wherein at least one of a decrease in the activity of NRF2, an increase in the activity of NF-κB, or a decrease in the activity of STAT3.

38-39. (canceled)
